(19) United States
(12) Patent Application Publication
(10) Pub. No.: US 2013/0203718 A1
(43) Pub. Date: Aug. 8, 2013

(54) PROGESTERONE RECEPTOR ANTAGONISTS AND USES THEREOF

(71) Applicants: Marie-Edith Rafestin-Oblin, Paris (FR); Mouad Alami, Chatenay Malabry (FR); Hugues Loosfelt, Le Kremlin Bicetre (FR); Abdallah Hamze, Chatenay Malabry (FR); Ali Junaid Khan, Le Kremlin Bicetre (FR); Abdellatif Tikad, Chatenay Malabry (FR); Marc Lombes, Le Kremlin Bicetre (FR); Jean-Daniel Brion, Chatenay Malabry (FR)

(72) Inventors: Marie-Edith Rafestin-Oblin, Paris (FR); Mouad Alami, Chatenay Malabry (FR); Hugues Loosfelt, Le Kremlin Bicetre (FR); Abdallah Hamze, Chatenay Malabry (FR); Ali Junaid Khan, Le Kremlin Bicetre (FR); Abdellatif Tikad, Chatenay Malabry (FR); Marc Lombes, Le Kremlin Bicetre (FR); Jean-Daniel Brion, Chatenay Malabry (FR)

(21) Appl. No.: 13/696,418

(22) PCT Filed: May 9, 2011
(86) PCT No.: PCT/EP2011/057387

§ 371 Date: Jan. 10, 2013

(30) Foreign Application Priority Data
May 7, 2010 (EP) 10305484.7

(51) Int. Cl. C07J 63/00; C07J 9/00; C07J 3/00; C07J 7/00; C07J 1/00; C07J 11/00; C07J 21/00

(52) U.S. Cl. CPC C07J 63/008; C07J 11/00; C07J 9/00; C07J 21/006; C07J 7/007; C07J 7/002; C07J 1/0011; C07J 7/0005; C07J 1/0022; C07J 3/00; C07J 1/0025
USPC 514/173; 514/169; 514/182; 514/177; 552/609; 552/555; 514/755; 570/130; 552/554; 552/556; 568/665; 514/715; 514/729; 568/817; 549/336; 514/462; 568/372; 514/691; 552/603

(57) ABSTRACT

The present invention relates to a compound of formula (I): for its use as progesterone receptor antagonist, in particular for its use for the prevention and/or the treatment of cancer or uterine pathologies.

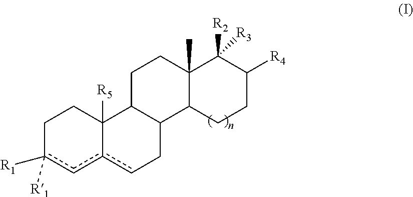

The present invention concerns novel progesterone receptor antagonists and uses thereof, in particular for the treatment of breast cancer.

Progesterone, secreted by ovaries and placenta, plays a major role in reproductive functions. This hormone acts through a nuclear receptor that belongs to the ligand-induced transcription factor family, the progesterone receptor (PR) (Loosfelt, H. et al. Proc. Natl. Acad. Sci. USA 1986, 83, 9045). Human PR is expressed as two isoforms, PRA (769 amino acids) and PRB (933 amino acids), alternatively transcribed from a unique gene. Both isoforms differ only by the size of their N-terminal region, but harbor distinct biological and transcriptional properties.

In the absence of ligand, PR exists within target cells in a transcriptionnally inactive form. Upon ligand binding, PR undergoes a substantial conformational change leading to its association, as a dimer, with hormone responsive elements (HRE) within target genes promoters. The DNA-bound receptor can then exert a positive or negative effect on gene by recruiting either co-activators or co-repressors. Co-activators positively regulate transcriptional efficacy by recruiting multiprotein complexes to DNA leading to chromatin remodelling and interaction with general transcription factors. Co-repressors recruited to the DNA-bound receptor facilitate chromatin condensation and silence transcription. Numerous transcriptional co-activators and co-repressors have been identified whose relative and absolute expression levels vary among cells.

Genomic targets of PRA and PRB include the key mediators of various cell signaling pathways (cell cycle, apoptosis, adhesion, growth factors etc) implicated in cancer (Richer, J. K., et al. J Biol Chem, 2002, 277, 5209; Jacobsen, B. M., et al. J Biol Chem, 2002, 277, 27793). Apart from classical genomic functions, PR isoforms are also capable of interacting with major cytoplasmic signaling pathways (Faivre, E. J. et al Mol Cell Biol, 2007, 27, 466) (Erk1/2 MAPK, EGF, Src etc.) frequently activated in cancer cells. Together, these targets play essential role in tissue proliferation, in particular through autocrine and paracrine mechanisms. Co-regulator recruitment as well as post-translational modifications of PR (phosphorylation, sumoylation, ubiquitination) are major determinants of transcriptional dynamics (Daniel, A. R. et al Mol Endocrinol 2007, 21, 2890). Dysregulation of PR isoforms transcriptional activities can therefore occur through abnormal expression of these co-regulating proteins in tumor cells.

Accumulating evidences based on various in vivo and in vitro studies suggest a major role of progestins in mammary carcinogenesis (Beleut, M., et al. Proc Natl Acad Sci USA 2010, 107, 2989; McGowan, E. M., et al., Cancer Res 2007, 67, 8942). Deregulation of the PRA/PRB expression ratio is often observed in breast and endometrial cancers. Given that their transcriptional activities as well as their genomic targets are somewhat different, any variation in PR isoforms expression would lead to hormonally sensitive changes in proliferation and invasiveness of tumor cells (Mote, P. A., et al. Breast Cancer Res Treat, 2002, 72, 163).

Previous data strongly suggest that PR should be considered as a major pharmacological target for prevention or treatment of PR-mediated diseases. Highly specific progesterone antagonist ligands able to by-pass PR interactions with co-regulating proteins are suitable to prevent deleterious effects on transcription regulation often observed with classical antagonists.

The steroidal PR antagonists available today, including RU486 (mifepristone), are molecules derived either from progesterone or testosterone. They are characterized by a C11-bulky substituent responsible for their antagonist character. Upon RU486 binding, the human PR undergoes a conformational change which is related but distinct from that triggered by progesterone. RU486 forms with PR a highly stable complex able to interact with DNA and to recruit transcriptional co-repressors. RU486 has been designed as “active antagonist”. It has a high efficacy to antagonize PR, but is not PR selective and interact with the androgen receptor (AR), the glucocorticoid receptor (GR) and to a much less extend to the mineralocorticoid receptor (MR). Furthermore, RU486 has a partial agonist activity which is related to its capacity to promote PR recruitment of transcriptional co-activators.

The aim of the present invention is to provide a novel class of progesterone receptor antagonists having new pharmacological properties.

In particular, the aim of the present invention is to provide selective progesterone receptor antagonists which do not interact with the androgen receptor, the glucocorticoid receptor and the mineralocorticoid receptor.

The aim of the present invention is also to provide full PR antagonists devoid of any agonist activity.

The present invention relates to a compound of formula (I):

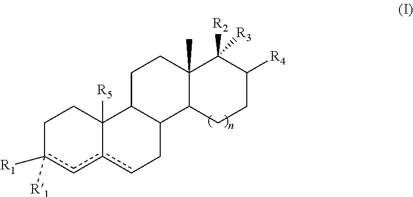

wherein
n is 0 or 1;
R₅ is H or CH₃;

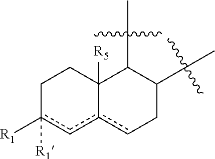

is selected from (Ia), (Ib), (Ic), (Id), (Ie), (If), (Ig) and (Ih):

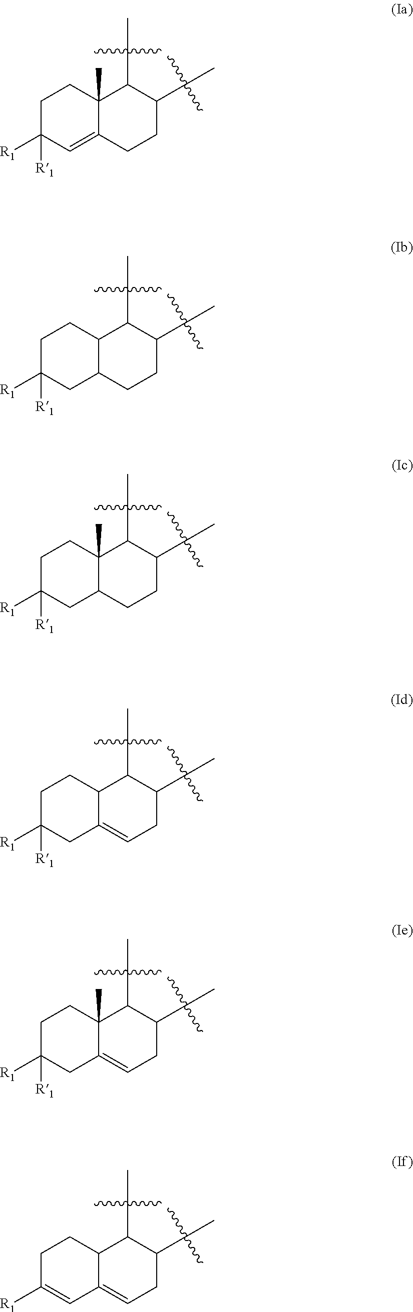

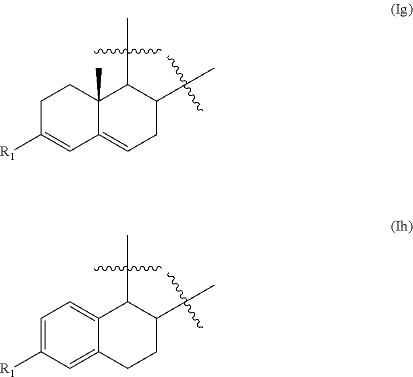

R₁ and R₁′ are each independently selected from H, OR₆, and halogen, or together with the carbon atom to which they are attached form a group C═O, or a 5 to 7 membered heterocyclyl group; provided that when

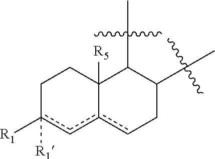

is (If), (Ig) or (Ih), R₁ cannot be C═O;
R₂ and R₃ are each independently selected from H, C(O)R₈, OR₇, halogen, (hetero)aryl, CH(OR₇)(R₈), C(OR₆)(C≡CR₆)(R₈) and C≡CR₆, or together with the carbon atom to which they are attached form a group C═O,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms;
   with the exclusion of the compound where

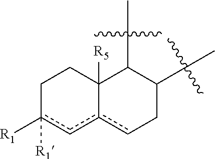

is (Ia), R₁ and R′₁ together with the carbon atom to which they are attached form a group C═O, n is O, R₃ and R₄ are H, and R₂ is COCH₃,
and the compound where

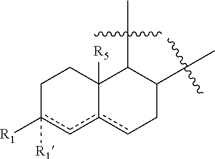

is (Ia), R₁ and R′₁ together with the carbon atom to which they are attached form a group C═O, n is 0, R₃ and R₄ are H, and R₂ is OH,
or its pharmaceutically acceptable salts, hydrates or hydrated salts or its polymorphic crystalline structures, racemates, diastereoisomers or enantiomers,

for its use as progesterone receptor antagonist, in particular for its use for estrogen-free contraception, emergency contraception, antigestation, or for its use as abortifacient, or for its use for the prevention and/or the treatment of pathologies involving progesterone receptor, in particular for the prevention and/or the treatment of cancer or uterine pathologies.

The term “alkyl” means a saturated or unsaturated aliphatic hydrocarbon group which may be straight or branched having 1 to 6 carbon atoms in the chain. “Branched” means that one or lower alkyl groups such as methyl, ethyl or propyl are attached to a linear alkyl chain. <<Lower alkyl>> means 1 to 4 carbon atoms in the chain which may be straight or branched. The alkyl may be substituted with one or more <<alkyl group substituents>> which may be the same or different, and include for instance halo, cycloalkyl, hydroxy (OH), alkoxy, amino (NH₂), acylamino (NHCOAlk), aroylamino (NHCOAr), carboxy (COOH).

The term “alkoxy” refers to an —O-alkyl radical.

The term “halo” or “halogen” refers to the atoms of the group 17 of the periodic table (halogens) and includes in particular fluorine, chlorine, bromine, and iodine atom.

The term “aryl” (or Ar) refers to an aromatic monocyclic, bicyclic, or tricyclic hydrocarbon ring system, wherein any ring atom capable of substitution may be substituted by a substituent. Examples of aryl moieties include, but are not limited to, phenyl, naphthyl, and anthracenyl. The term “aryl” also includes “heteroaryl” which refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution may be substituted by a substituent.

The term “heterocyclyl” refers to a nonaromatic 5-7 membered monocyclic, ring system having 1-3 heteroatoms, said heteroatoms being selected from O, N, or S (e.g., carbon atoms and 1-3 heteroatoms of N, O, or S), wherein any ring atom capable of substitution may be substituted by a substituent.

The compounds herein described may have asymmetric centers. Compounds of the present invention containing an asymmetrically substituted atom may be isolated in optically active or racemic forms. It is well-known in the art how to prepare optically active forms, such as by resolution of racemic forms or by synthesis from optically active starting materials. All chiral, diastereomeric, racemic forms and all geometric isomeric forms of a compound are intended, unless the stereochemistry or the isomeric form is specifically indicated.

“Pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “pharmaceutically acceptable salt” refers to salts which retain the biological effectiveness and properties of the compounds of the invention and which are not biologically or otherwise undesirable. In many cases, the compounds of the invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). The expression “non-toxic pharmaceutically acceptable salts” refers to non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

In the context of the invention, the term “treating” or “treatment”, as used herein, means reversing, alleviating, inhibiting the progress of, or preventing the disorder or condition to which such term applies, or one or more symptoms of such disorder or condition.

While it is possible for the compounds of the invention having formula (I) to be administered alone it is preferred to present them as pharmaceutical compositions. The pharmaceutical compositions, both for veterinary and for human use, useful according to the present invention comprise at least one compound having formula (I) as above defined, together with one or more pharmaceutically acceptable carriers and optionally other therapeutic ingredients.

In certain preferred embodiments, active ingredients necessary in combination therapy may be combined in a single pharmaceutical composition for simultaneous administration.

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules, suppositeries, patches, vaginal ring, intra uterine delivery.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The pharmaceutical compositions can be administered in a suitable formulation to humans and animals by topical or systemic administration, including oral, rectal, nasal, buccal, ocular, sublingual, transdermal, topical, vaginal, enteral, parenteral (including subcutaneous, intra-arterial, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Total daily dose of the compounds of the invention administered to a subject in single or divided doses may be in amounts, for example, of from about 0.001 to about 100 mg/kg body weight daily and preferably 0.01 to 10 mg/kg/day. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

The above applications/uses of compounds having formula (I) are based on the fact that these compounds are able to bind to PR, thus ensuring a competition

with progesterone, but leading to unstable PR complexes unable to recruit transcriptional co-regulators and devoid of any specific interaction with ligand-induced molecular partner. Such newly generated molecules constitute a novel class of PR antagonists and may be therefore referred as to “passive antagonists”.

Such specific progesterone antagonists may be used as pharmacological tools for breast and endometrial cancer therapies. They can also be used to treat, prevent or alleviate proliferative endometrium diseases such as myomas and endometriosis. Furthermore, PR antagonists can be used for emergency contraception and long term estrogen-free contraception.

The compounds of the invention have an antiprogestin and antigonadotrope activity. Thus, they may be used for the following applications: contraception (estrogen-free contraception, emergency contraception), antigestation, abortifacient, management of early in utero foetal demise, prepartum cervical maturation.

These compounds may also be used for the treatment and/or the prevention of cancers involving PR (progesterone receptor). Among these cancers, one may cite cancers of breast, endometrium, ovaries, central nervous system, lungs, to pituitary.

The present invention also relates to compounds of formula (I) as defined above for their use for the prevention and/or the treatment of uterine pathologies, such as endometriosis, myomas or dysfunctional bleeding.

The present invention also relates to compounds of formula (I) as defined above for their use for the prevention and/or the treatment of hirsutism.

The compounds of formula (I) may also be used in cosmetic compositions for treating the skin or hair. The present invention also relates to a method of cosmetic treatment comprising the application of a compound of formula (I) on skin or hair.

Preferably, the present invention relates to compounds of formula (I) as defined above for their use for the prevention and/or the treatment of breast cancer.

The present invention relates to a compound of formula (I):

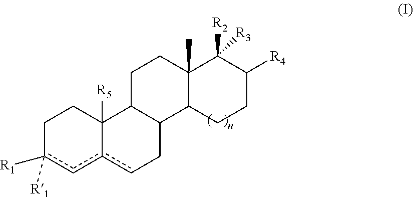

wherein
n is 0 or 1;
R₅ is H or CH₃;

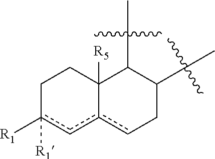

is selected from (Ia), (Ib), (Ic), (Id), (Ie), (If) and (Ig):

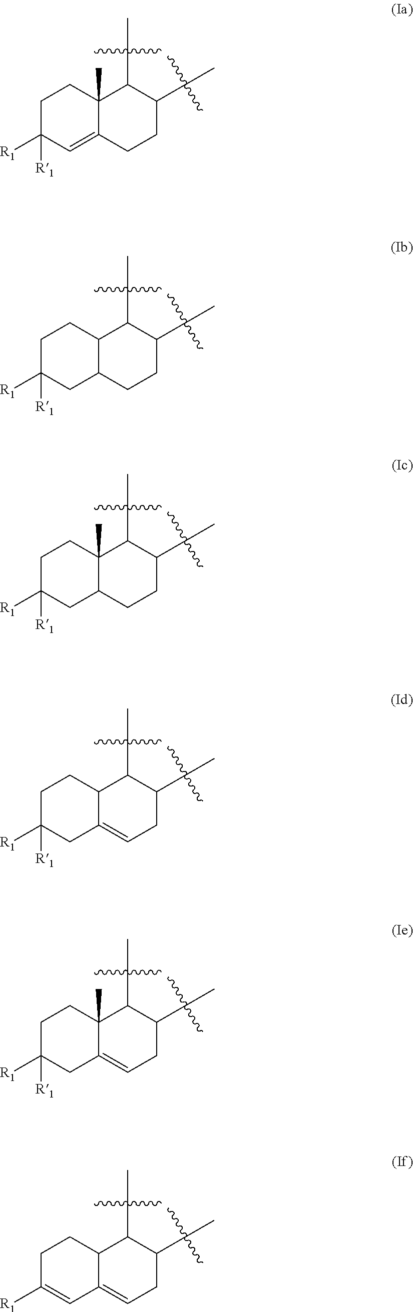

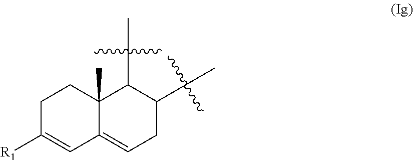

R₁ and R₁′ are each independently selected from H, OR₆, and halogen, or together with the carbon atom to which they are attached form a group C═O, or a 5 to 7 membered heterocyclyl group;

provided that when

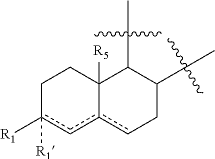

is (If) or (Ig), R₁ cannot be C═O;
R₂ and R₃ are each independently selected from H, C(O)R₈, OR₇, halogen, CH(OR₇)(R₈), C(OR₆)(C≡CR₆)(R₈) and C≡CR₆, or together with the carbon atom to which they are attached form a group C═O,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms;
   with the exclusion of the compound where

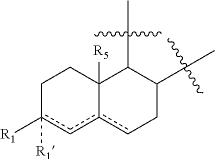

is (Ia), R₁ and R′₁ together with the carbon atom to which they are attached form a group C═O, n is 0, R₃ and R₄ are H, and R₂ is COCH₃, and the compound where

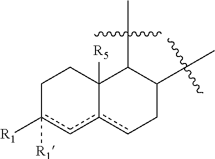

is (Ia), R₁ and R′₁ together with the carbon atom to which they are attached form a group C═O, n is 0, R₃ and R₄ are H, and R₂ is OH,
or its pharmaceutically acceptable salts, hydrates or hydrated salts or its polymorphic crystalline structures, racemates, diastereoisomers or enantiomers,

for its use as progesterone receptor antagonist, in particular for its use for estrogen-free contraception, emergency contraception, antigestation, or for its use as abortifacient, or for its use for the prevention and/or the treatment of pathologies involving progesterone receptor, in particular for the prevention and/or the treatment of cancer or uterine pathologies.

The present invention relates to a compound of formula (I):

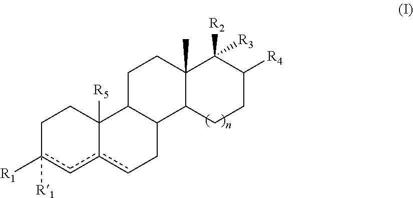

wherein
n is 0 or 1;

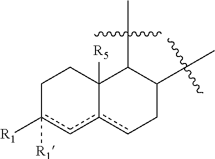

is selected from (Ia), (Ib), (Ic), (Id), (Ie), (If) and (Ig):

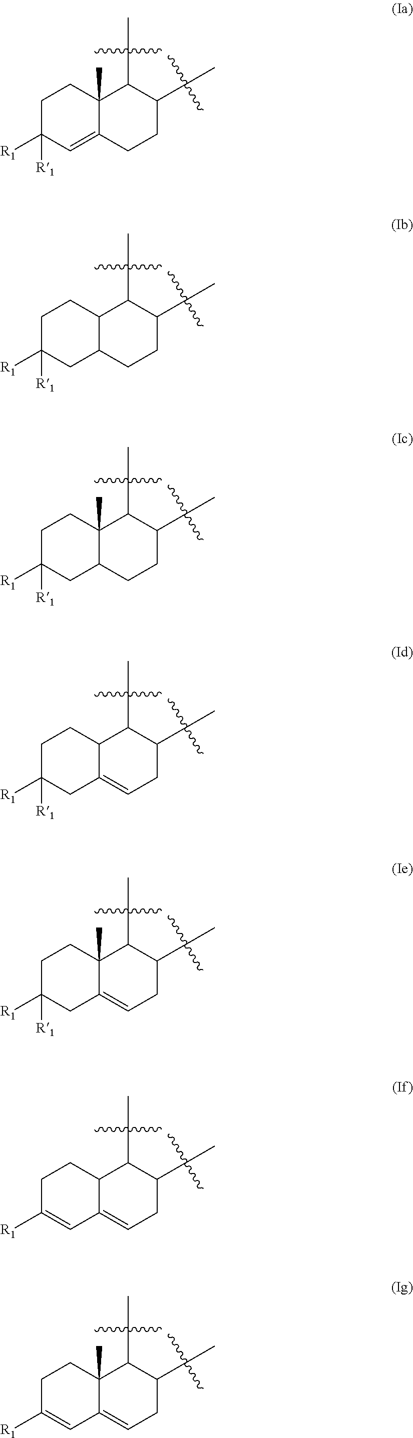

R₁ and R₁′ are each independently selected from H, OR₆, and halogen, or a to 7 membered heterocyclyl group, preferably from H and halogen;
R₂ and R₃ are each independently selected from H, C(O)R₈, OR₇, halogen, CH(OR₇)(R₈), C(OR₆)(C≡CR₆)(R₈) and C≡CR₆,

provided that when R₂ is OH, R₃ cannot be H,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms; or its pharmaceutically acceptable salts, hydrates or hydrated salts or its polymorphic crystalline structures, racemates, diastereoisomers or enantiomers, with the exclusion of the compounds:

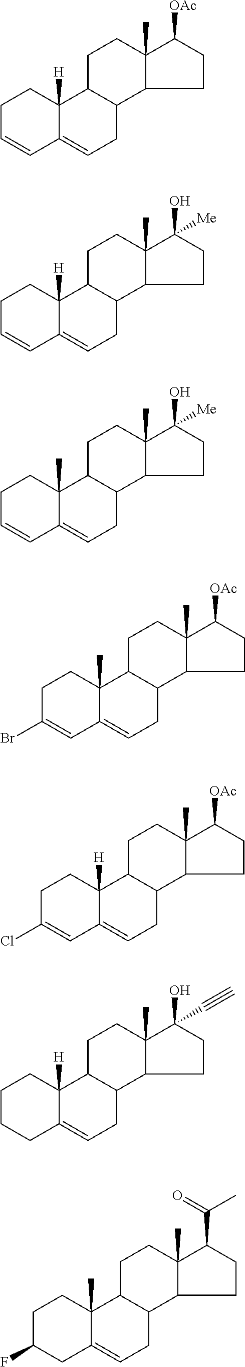

for its use as progesterone receptor antagonist, in particular for its use for estrogen-free contraception, emergency contraception, antigestation, or for its use as abortifacient, or for its use for the prevention and/or the treatment of pathologies involving progesterone receptor, in particular for the prevention and/or the treatment of cancer or uterine pathologies.

The present invention relates to a compound of formula (I) wherein:
n is 0 or 1;

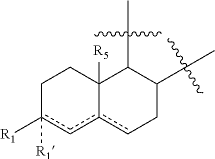

is selected from (Ia), (Ib), (Ic), (Id), (Ie), (If) and (Ig):

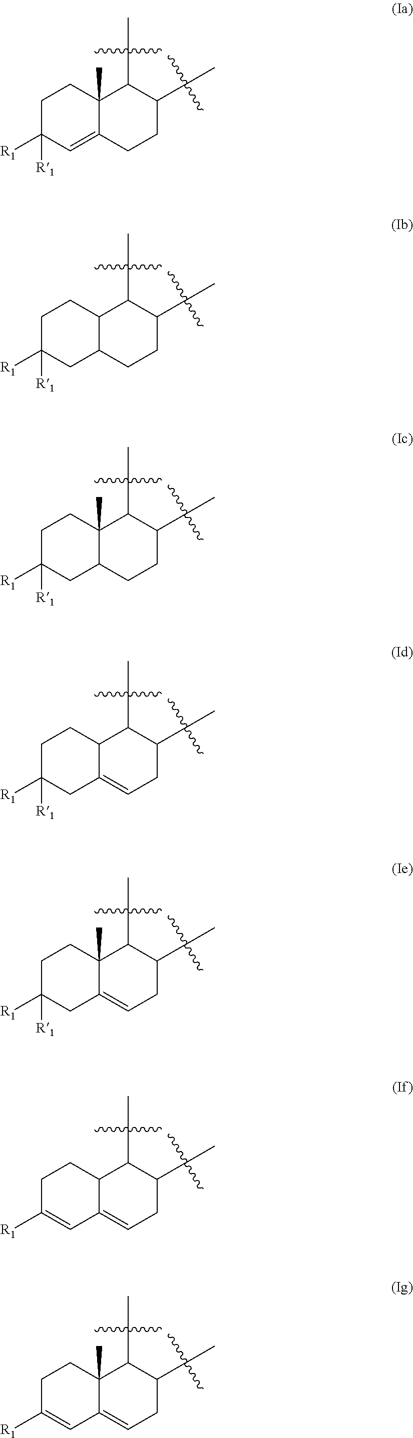

R₁ and R₁′ are each independently selected from H, OR₆, and halogen, or a to 7 membered heterocyclyl group, preferably from H and halogen;
R₂ and R₃ are each independently selected from H, C(O)R₈, OR₇, halogen, CH(OR₇)(R₈), C(OR₆)(C≡CR₆)(R₈) and C≡CR₆,

provided that when R₂ is OH, R₃ cannot be H,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms; or its pharmaceutically acceptable salts, hydrates or hydrated salts or its polymorphic crystalline structures, racemates, diastereoisomers or enantiomers,

for its use for the prevention and/or the treatment of pathologies involving progesterone receptor, in particular for the prevention and/or the treatment of cancer or uterine pathologies.

The present invention relates to a compound of formula (I) wherein:
n is 0 or 1;

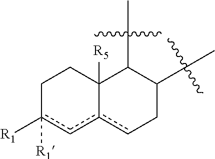

is selected from (Ia), (Ib), (Ic), (Id), (Ie), (If) and (Ig):

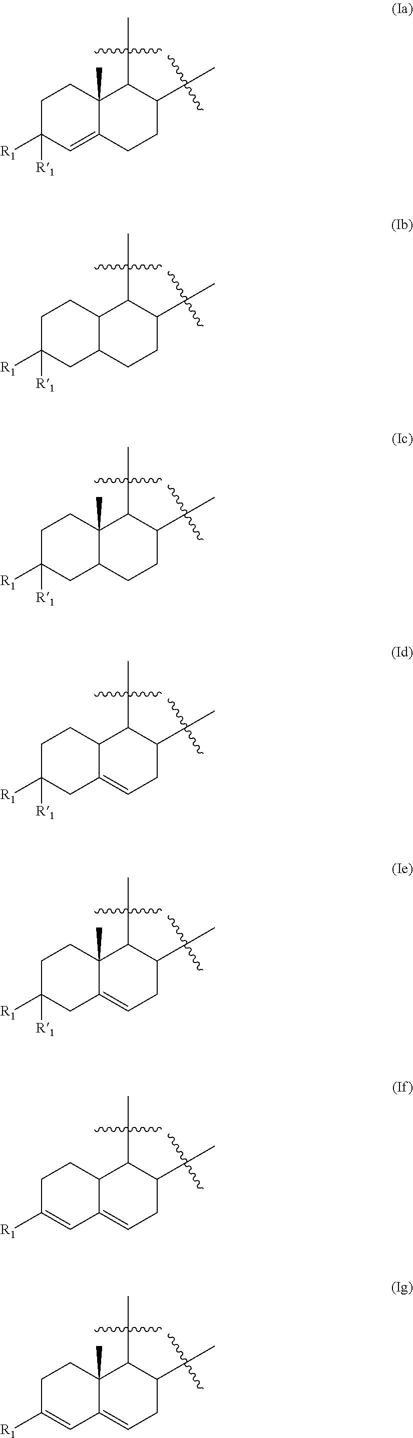

R₁ and R₁′ are each independently selected from H, OR₆, and halogen, or a to 7 membered heterocyclyl group, preferably from H and halogen;
R₂ and R₃ are each independently selected from H, C(O)R₈, OR₇, halogen, CH(OR₇)(R₈), C(OR₆)(C≡CR₆)(R₈) and C≡CR₆,

provided that when R₂ is OH, R₃ cannot be H,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms; or its pharmaceutically acceptable salts, hydrates or hydrated salts or its polymorphic crystalline structures, racemates, diastereoisomers or enantiomers, with the exclusion of the compounds:

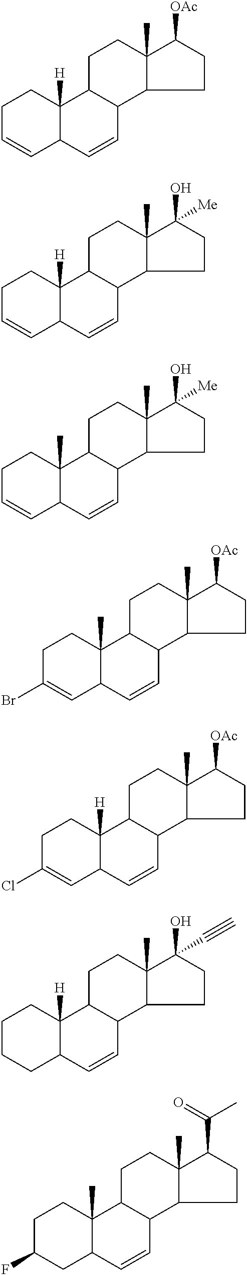

for its use as progesterone receptor antagonist, in particular for its use for estrogen-free contraception, emergency contraception, antigestation, or for its use as abortifacient.

According to a particular embodiment, the present invention relates to the compound of formula (I) for its use as defined above, with the exclusion of the compound where

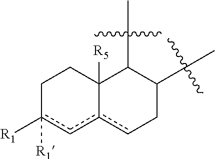

### is (Ic), R₁ and R′₁ are H, n is 0, R₃ and R₄ are H, and R₂ is COCH₃.

In formula (I), the alkyl groups are preferably methyl groups.

In formula (I), the halogen groups are preferably fluorine groups.

Preferably, in formula (I), when R₁ and R′₁ together with the carbon atom to which they are attached form a 5 to 7 membered heterocyclyl group, said heterocyclyl group is a group of formula

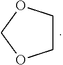

Preferably, in formula (I), R₁ and R₁′ are each independently selected from H and halogen.

According to an embodiment, the present invention relates to the compound of formula (I) for its use as defined above, wherein R₃ is H and R₂ is selected from C(O)R₈, OR₇, halogen, CH(OR₇)(R₈), C(OR₆)(C≡CR₆)(R₈) and C≡CR₆.

According to an embodiment, the present invention relates to the compound of formula (I) for its use as defined above, wherein R₃ is H and R₂ is selected from C(O)R₈, OR₁₇, halogen, CH(OR₇)(R₈), C(OR₆)(C≡CR₆)(R₈) and C≡CR₆, wherein:

R₆ is H or an alkyl group comprising from 1 to 6 carbon atoms;

R₇ is H or an alkyl group comprising from 1 to 6 carbon atoms, or a group C(O)R₉,

R′₇ is an alkyl group comprising from 1 to 6 carbon atoms, or a group C(O)R₉,

R₈ is an alkyl group comprising from 1 to 6 carbon atoms; and

R₉ is an alkyl group comprising from 1 to 6 carbon atoms.

Preferably, in formula (I), R₃ is H and R₂ is OH or OAc.

Preferably, in formula (I), R₃ is H and R₂ is OAc.

Preferably, R₆ and R₇ are H and R₈ is methyl.

According to another embodiment, the present invention relates to the compound of formula (I) for its use as defined above, wherein R₃ is H and R₂ is selected from COCH₃, CH(CH₃)(OH) and CH(CH₃)(OAc).

According to another embodiment, the present invention relates to the compound of formula (I) for its use as defined above, wherein R₃ is H and R₂ is C(C≡CH)(CH₃)(OH).

According to another embodiment, the present invention relates to the compound of formula (I) for its use as defined above, wherein R₂ is OH and R₃ is C≡CR₆, R₆ being preferably H or CH₃.

According to another embodiment, the present invention relates to the compound of formula (I) for its use for the prevention and/or the treatment of pathologies involving progesterone receptor, in particular for the prevention and/or the treatment of cancer or uterine pathologies, wherein R₂ is OH and R₃ is C≡CR₆, R₆ being preferably H or CH₃, and advantageously R₆ being CH₃.

The present invention also relates to the compound of formula (I) for its use as defined above, wherein n is 0 and R₁ and R′₁ are H.

Such compounds have the following formula (V):

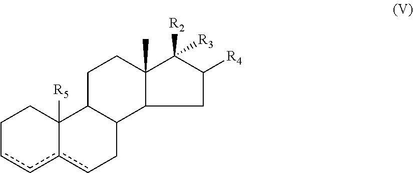

wherein R₂, R₃, R₄ and R₅ are as defined above in formula (I).

Preferred compounds of formula (V) are compounds having formula (V-1) as follows:

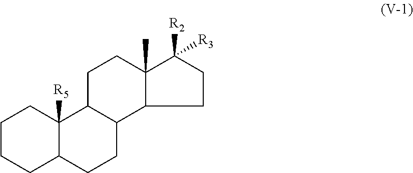

According to a particular embodiment, in formula (V-1), R₃ is H.

According to a particular embodiment, in formula (V-1), R₂ is OR₇, R₇ being preferably H or an alkyl group. Most preferably, R₂ is OH.

According to a particular embodiment, in formula (V-1), R₂ is OR₇, R₇ being preferably H or an alkyl group, and R₃ is H. Most preferably, R₂ is OH and R₃ is H.

According to a particular embodiment, in formula (V-1), R₂ is OR₇, R₇ being preferably H or an alkyl group, and R₃ is C≡CR₆, R₆ being preferably H or CH₃. Most preferably, R₂ is OH and R₃ is C≡CH or C≡C≡CH₃.

According to a particular embodiment, in formula (V-1), R₂ and R₃ form a group C═O together with the carbon atom to which they are attached.

According to a particular embodiment, in formula (V-1), R₃ is H, and R₂ is C(O)R₈ or CH(OR₇)(R₈), R₇ and R₈ being as defined above, R₇ being preferably H or COCH₃ and R₈ being preferably CH₃.

Preferred compounds of formula (V) are as follows:

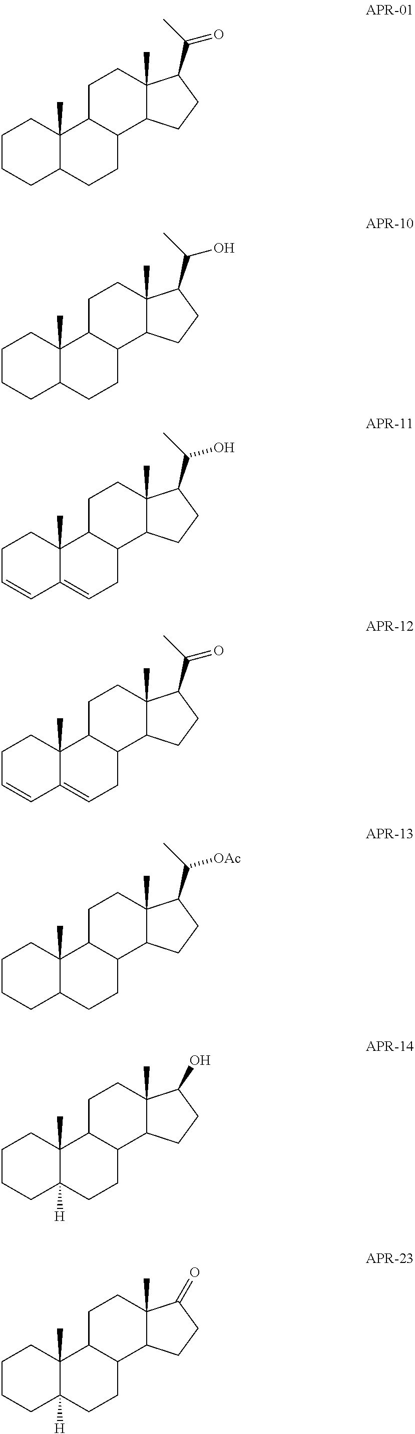

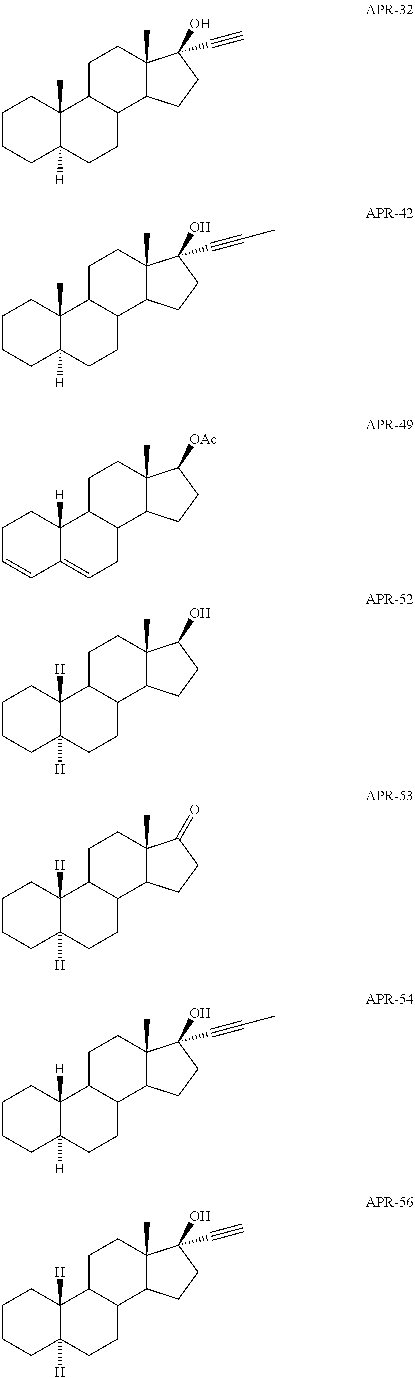

The present invention also relates to the compound of formula (II):

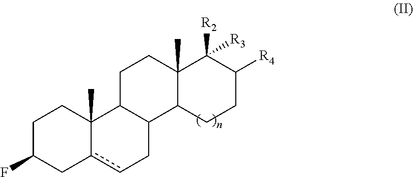

wherein n, R₂, R₃, and R₄ are as defined above in formula (I),

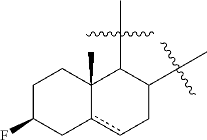

is selected from (IIa), and (IIb):

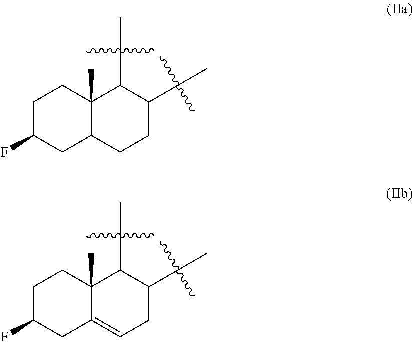 [SMILES: *C1CCC2C[C@@H](F)CC[C@]2(C)C1* ; *C1CC=C2C[C@@H](F)CC[C@]2(C)C1*]

for its use as defined above, as progesterone receptor antagonist, in particular for its use for the prevention and/or the treatment of breast cancer.

The present invention also relates to the compound of formula (I′):

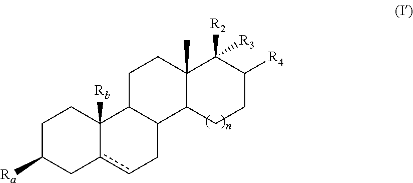

wherein n, R₂, R₃, and R₄ are as defined above, and

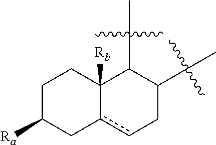

is selected from (IIa′), (IIb′), (IIc′) and (IId′):

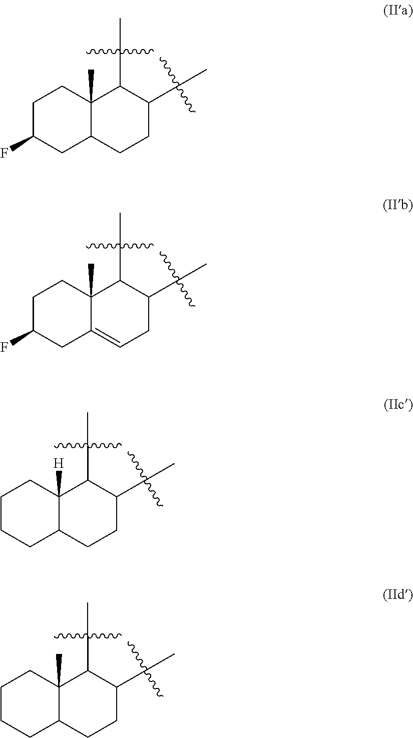 [SMILES: *C1CCC2C[C@@H](F)CC[C@]2(C)C1* ; *C1CC=C2C[C@@H](F)CC[C@]2(C)C1* ; *C1CCC2CCCC[C@@H]2C1* ; *C1CCC2CCCC[C@]2(C)C1*]

for its use for the prevention and/or the treatment of pathologies involving progesterone receptor, in particular for the prevention and/or the treatment of cancer or uterine pathologies.

The present invention also relates to the compound of formula (II):

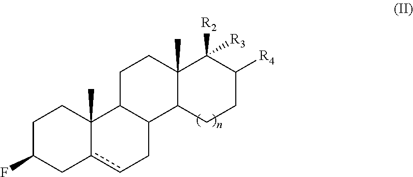

wherein n is 0 or 1,

R₂ and R₃ are each independently selected from H, C(O)R₈, OR₇, halogen, CH(OR₇)(R₈), C(OR₆)(C≡CR₆)(R₈) and C≡CR₆,

provided that when R₂ is OH, R₃ cannot be H,
R₄ is H or an alkyl group comprising from 1 to 6 carbon atoms;

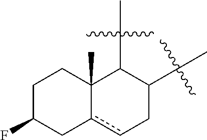

is selected from (IIa), and (IIb):

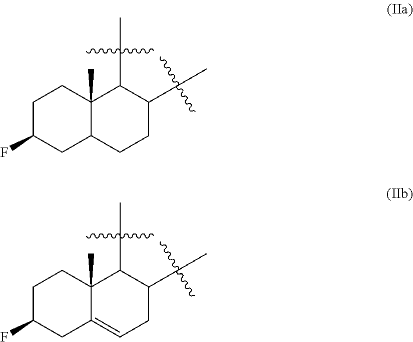 [SMILES: *C1CCC2C[C@@H](F)CC[C@]2(C)C1* ; *C1CC=C2C[C@@H](F)CC[C@]2(C)C1*]

for its use for the prevention and/or the treatment of pathologies involving progesterone receptor, in particular for the prevention and/or the treatment of cancer or uterine pathologies.

Compounds of formula (II) are compounds having formula (I) wherein R₁ is F and R′₁ is H.

Preferred compounds of formula (II) are as follows:

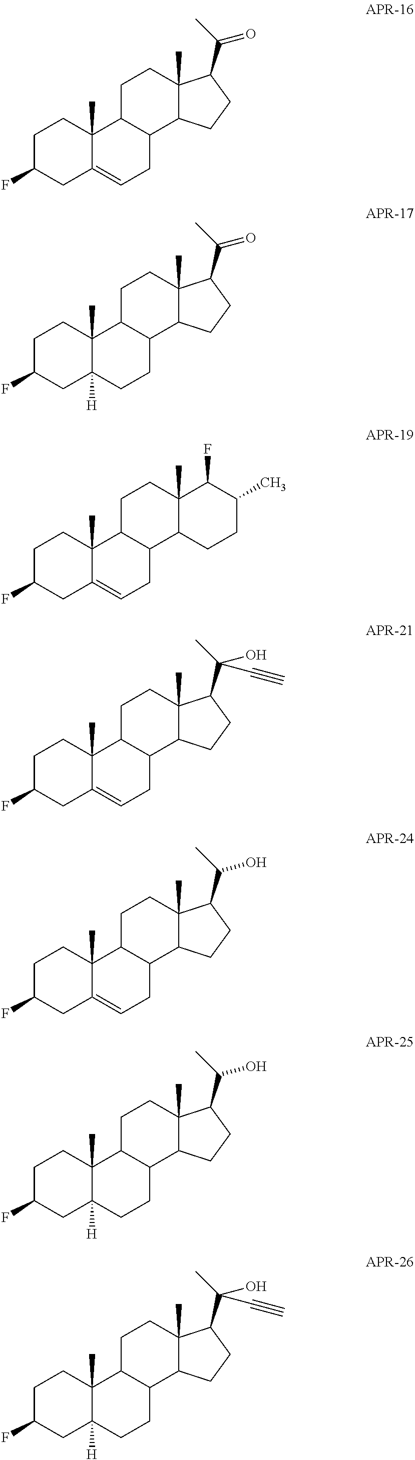

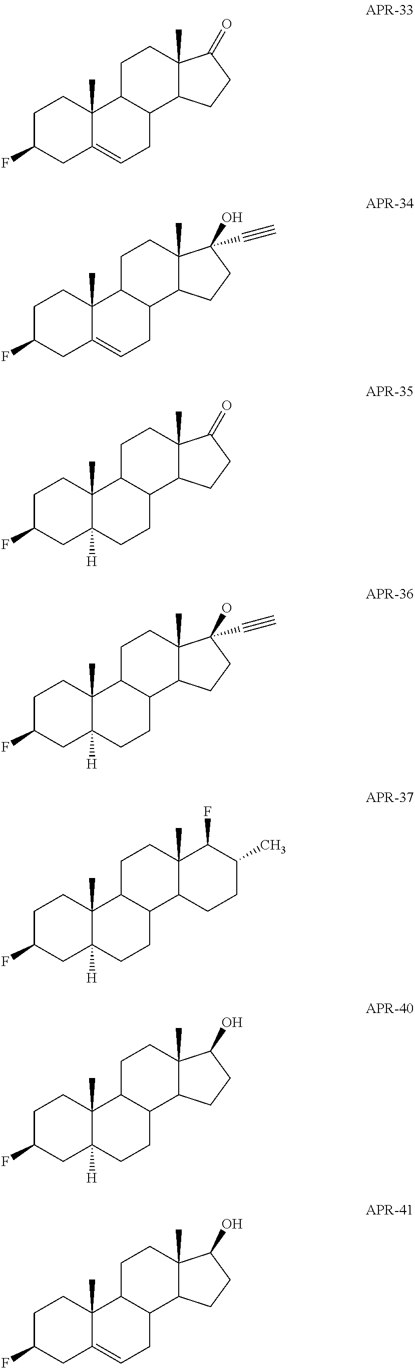

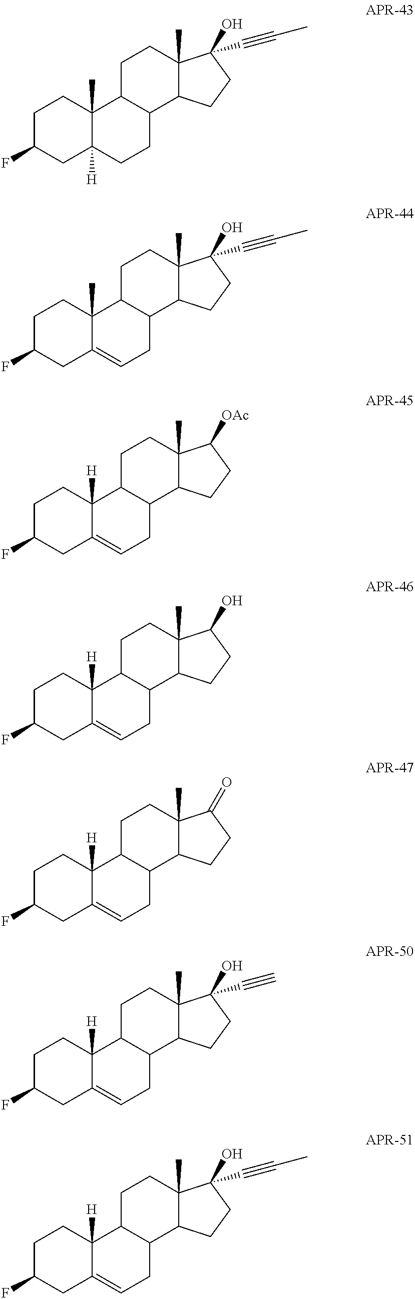

The present invention also relates to the compound of formula (II-1):

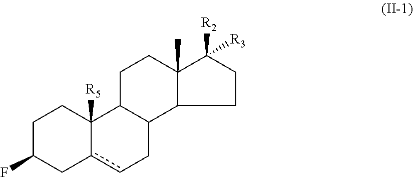

wherein R₅, R₂ and R₃ are as defined above in formula (I),

and

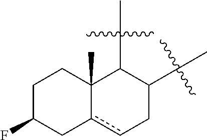

is as defined above,

for its use as defined above, as progesterone receptor antagonist, in particular for its use for the prevention and/or the treatment of breast cancer.

A particular group of compounds of formula (II-1) are compounds having formula (II-1-1) as follows:

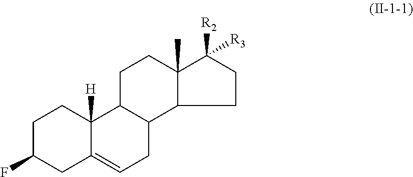

wherein R₂ and R₃ are as defined above in formula (I).

According to a particular embodiment, in formula (II-1-1), R₃ is H.

According to a particular embodiment, in formula (II-1-1), R₂ is OR₇, R₇ being preferably H or a C(O)R₈ group, R₈ being preferably CH₃. Most preferably, R₂ is OH or OCH₃.

According to a particular embodiment, in formula (II-1-1), R₂ is OR₇, R₇ being preferably H, and R₃ is C≡CR₆, R₆ being preferably H or CH₃. Most preferably, R₂ is OH and R₃ is C≡CH or C≡CCH₃.

According to a particular embodiment, in formula (II-1-1), R₂ and R₃ form a group C═O together with the carbon atom to which they are attached.

A particular group of compounds of formula (II-1) are compounds having formula (II-1-2) as follows:

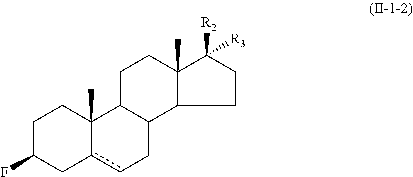

wherein R₂ and R₃ are as defined above in formula (I), and

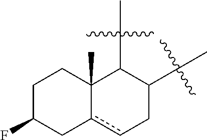

is (IIa) or (IIb) as defined above.

According to a particular embodiment, in formula (II-1-2), R₃ is H.

According to a particular embodiment, in formula (II-1-2), R₂ is OR₇, R₇ being preferably H or an alkyl group. Most preferably, R₂ is OH.

According to a particular embodiment, in formula (II-1-2), R₂ is OR₇, R₇ being preferably H, and R₃ is H. Most preferably, R₂ is OH and R₃ is H.

According to a particular embodiment, in formula (II-1-2), R₂ is OR₇, R₇ being preferably H, and R₃ is C≡CR₆, R₆ being preferably H or CH₃. Most preferably, R₂ is OH and R₃ is C═CH or C≡CCH₃.

According to a particular embodiment, in formula (II-1-2), R₂ and R₃ form a group C═O together with the carbon atom to which they are attached.

According to a particular embodiment, in formula (II-1-2), R₃ is H, and R₂ is selected from C(O)R₈, CH(OR₇)(R₈), and C(OR₇)(C≡CR₆)(R₈), R₇ and R₈ being as defined above, R₆ and R₇ being preferably H and R₈ being preferably CH₃.

The present invention also relates to the compound of formula (II-2′):

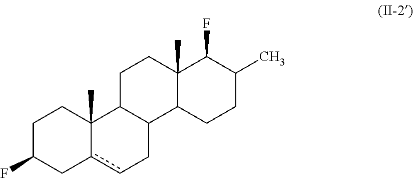

wherein

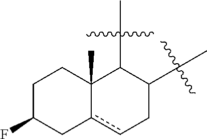

is as defined above,

for its use as defined above, as progesterone receptor antagonist, in particular for its use for the prevention and/or the treatment of breast cancer.

The present invention also relates to the compound of formula (II-2′):

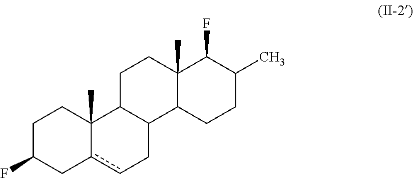

wherein

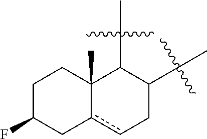

is as defined above,

for its use for the prevention and/or the treatment of pathologies involving progesterone receptor, in particular for the prevention and/or the treatment of cancer or uterine pathologies.

The present invention also relates to the compound of formula (III):

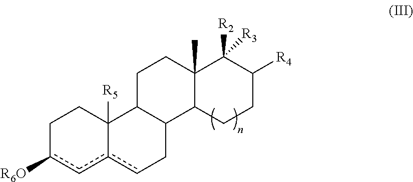

wherein n, R₂, R₃, R₄, R₅ and R₆ are as defined above in formula (I),

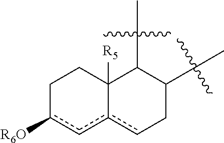

is selected from (IIIa), (IIIb), (IIIc), (IIId), (IIIe), (IIIf) and (IIIg):

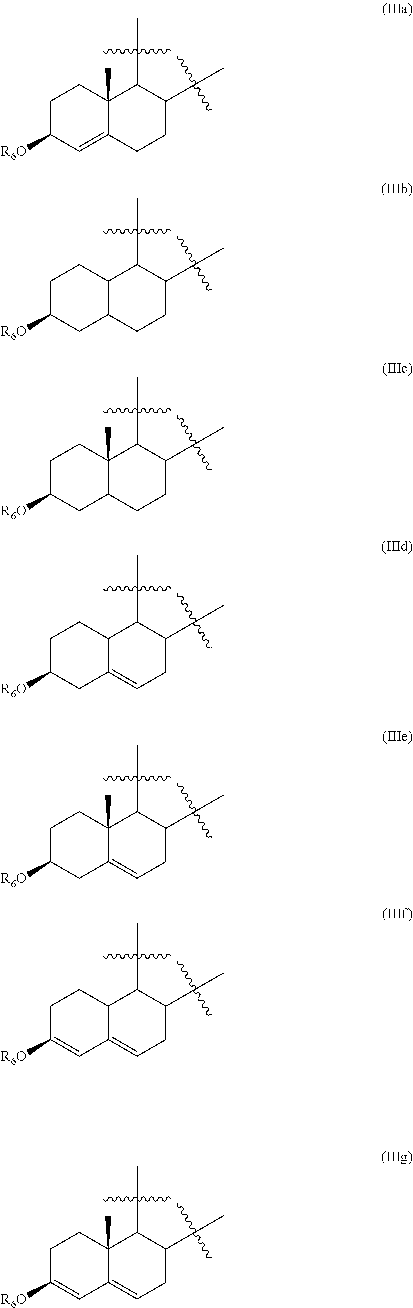

for its use as defined above, as progesterone receptor antagonist, in particular for its use for the prevention and/or the treatment of breast cancer.

Preferably, in formula (III) as defined above, R₆ is H or methyl.

The present invention also relates to the compound of formula (III-1):

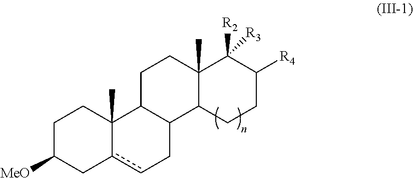

wherein n, R₂, R₃, and R₄ are as defined above in formula (I),

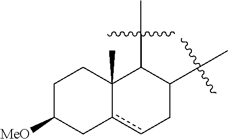

is selected from (III-1-a) and (III-1-b):

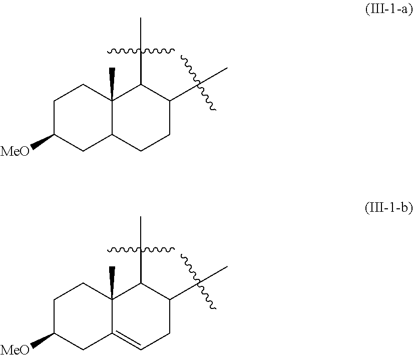 [SMILES: *C1CCC2C[C@@H](OC)CC[C@]2(C)C1* ; *C1CC=C2C[C@@H](OC)CC[C@]2(C)C1*]

for its use as defined above, as progesterone receptor antagonist, in particular for its use for the prevention and/or the treatment of breast cancer.

Preferred compounds of formula (III-1) are as follows:

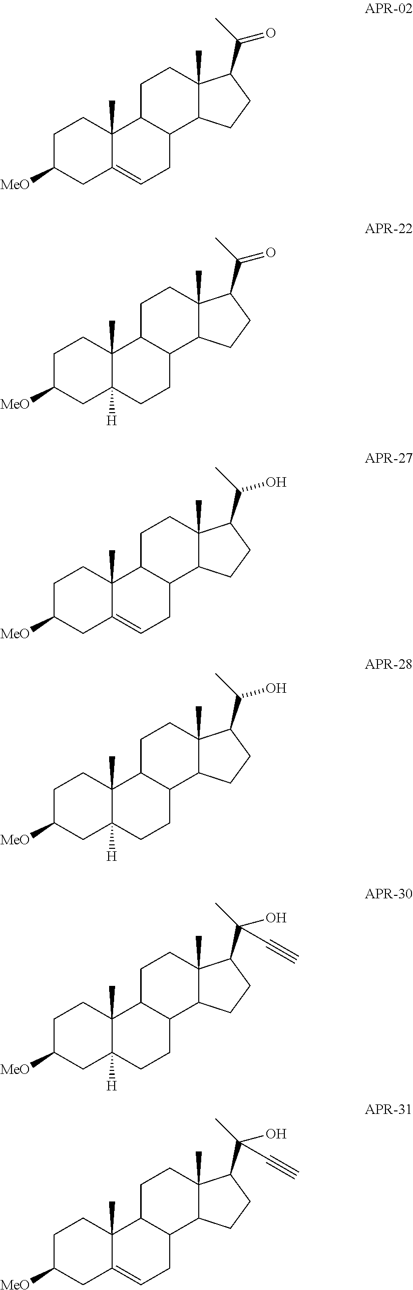 [SMILES: CO[C@H]1CC[C@@]2(C)C(=CCC3C2CC[C@@]2(C)C3CC[C@@H]2C(C)=O)C1 ; CO[C@H]1CC[C@]2(C)C3CC[C@@]4(C)C(CC[C@@H]4C(C)=O)C3CC[C@H]2C1 ; CO[C@H]1CC[C@@]2(C)C(=CCC3C2CC[C@@]2(C)C3CC[C@@H]2[C@H](C)O)C1 ; CO[C@H]1CC[C@]2(C)C3CC[C@@]4(C)C(CC[C@@H]4[C@H](C)O)C3CC[C@H]2C1 ; C#CC(C)(O)[C@H]1CCC2C3CC[C@H]4C[C@@H](OC)CC[C@]4(C)C3CC[C@@]21C ; C#CC(C)(O)[C@H]1CCC2C3CC=C4C[C@@H](OC)CC[C@]4(C)C3CC[C@@]21C]

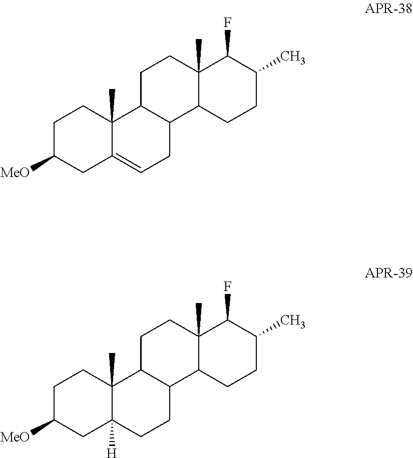 [SMILES: CO[C@H]1CC[C@@]2(C)C(=CCC3C2CC[C@@]2(C)C3CC[C@@H](C)[C@@H]2F)C1 ; CO[C@H]1CC[C@]2(C)C3CC[C@@]4(C)C(CC[C@@H](C)[C@@H]4F)C3CC[C@H]2C1]

The present invention also relates to the compound of formula (III-2):

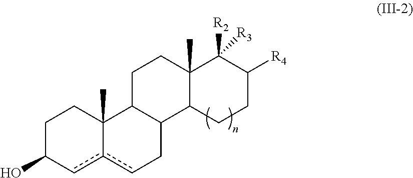

wherein n, R₂, R₃, and R₄ are as defined above in formula (I),

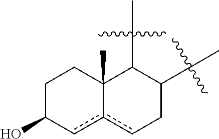

is selected from (III-2-a), (III-2-b), and (III-2-c):

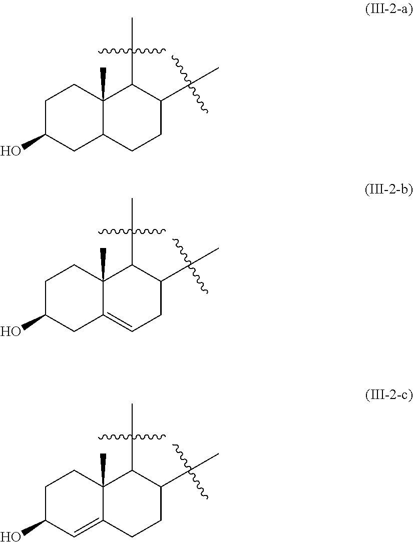 [SMILES: *C1CCC2C[C@@H](O)CC[C@]2(C)C1* ; *C1CC=C2C[C@@H](O)CC[C@]2(C)C1* ; *C1CCC2=C[C@@H](O)CC[C@]2(C)C1*]

for its use as defined above, as progesterone receptor antagonist, in particular for its use for the prevention and/or the treatment of breast cancer.

Preferred compounds of formula (III-2) are as follows:

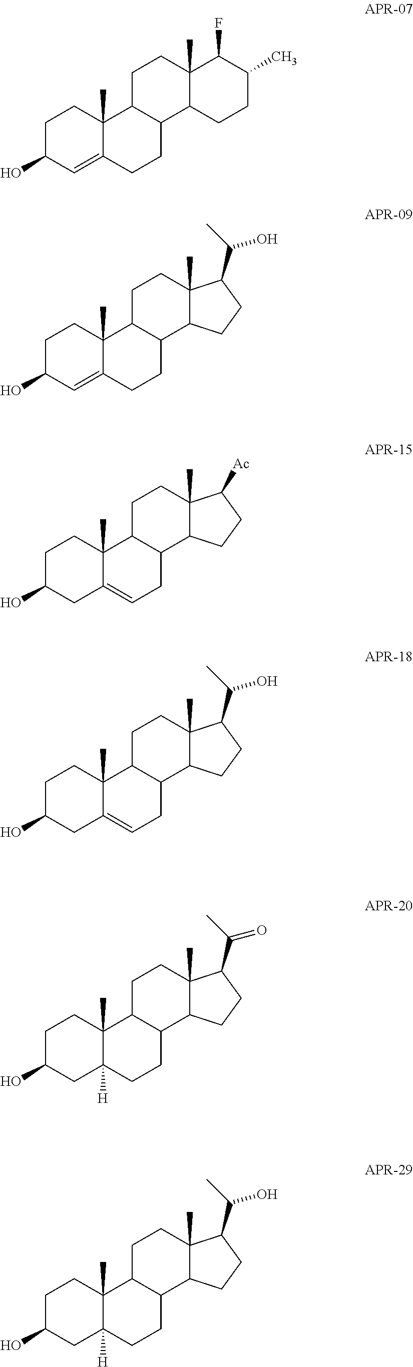 [SMILES: C[C@@H]1CCC2C3CCC4=C[C@@H](O)CC[C@]4(C)C3CC[C@]2(C)[C@H]1F ; C[C@H](O)[C@H]1CCC2C3CCC4=C[C@@H](O)CC[C@]4(C)C3CC[C@@]21C ; CC(=O)[C@H]1CCC2C3CC=C4C[C@@H](O)CC[C@]4(C)C3CC[C@@]21C ; C[C@H](O)[C@H]1CCC2C3CC=C4C[C@@H](O)CC[C@]4(C)C3CC[C@@]21C ; CC(=O)[C@H]1CCC2C3CC[C@H]4C[C@@H](O)CC[C@]4(C)C3CC[C@@]21C ; C[C@H](O)[C@H]1CCC2C3CC[C@H]4C[C@@H](O)CC[C@]4(C)C3CC[C@@]21C]

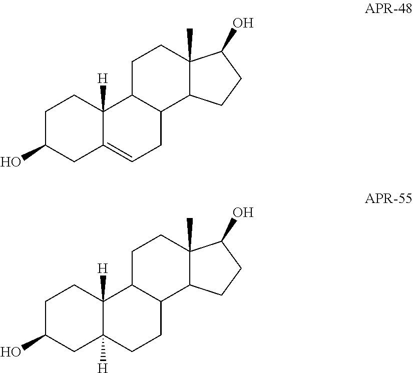 [SMILES: C[C@]12CCC3C(CC=C4C[C@@H](O)CC[C@@H]43)C1CC[C@@H]2O ; C[C@]12CCC3C(CC[C@H]4C[C@@H](O)CC[C@H]34)C1CC[C@@H]2O]

The present invention also relates to the compound of formula (IV):

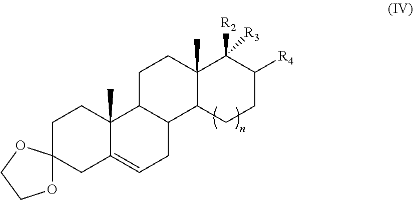

wherein n, R₂, R₃, and R₄ are as defined above in formula (I),

for its use as defined above, as progesterone receptor antagonist, in particular for its use for the prevention and/or the treatment of breast cancer.

Preferred compounds of formula (IV) are as follows:

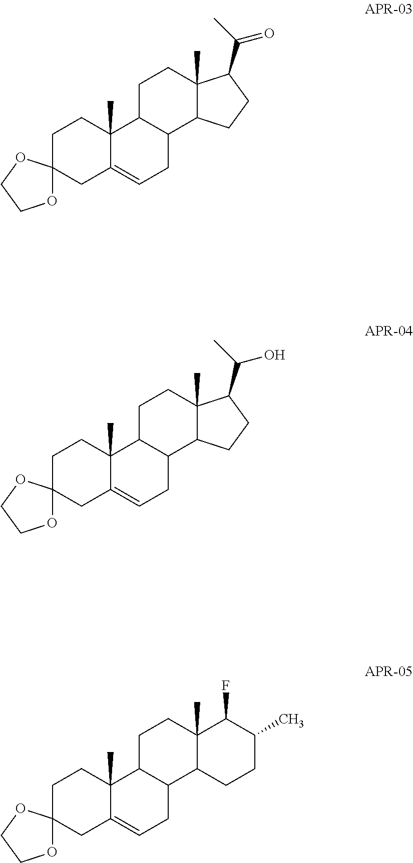 [SMILES: CC(=O)[C@H]1CCC2C3CC=C4CC5(CC[C@]4(C)C3CC[C@@]21C)OCCO5 ; CC(O)[C@H]1CCC2C3CC=C4CC5(CC[C@]4(C)C3CC[C@@]21C)OCCO5 ; C[C@@H]1CCC2C3CC=C4CC5(CC[C@]4(C)C3CC[C@]2(C)[C@H]1F)OCCO5]

The present invention also relates to a compound of formula (II-2):

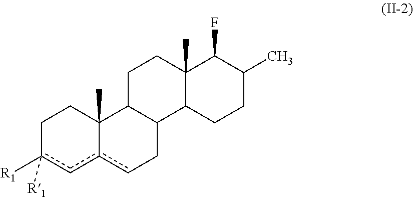

wherein:

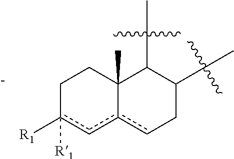

is selected from (II-2a), (II-2b), (II-2c), and (II-2d):

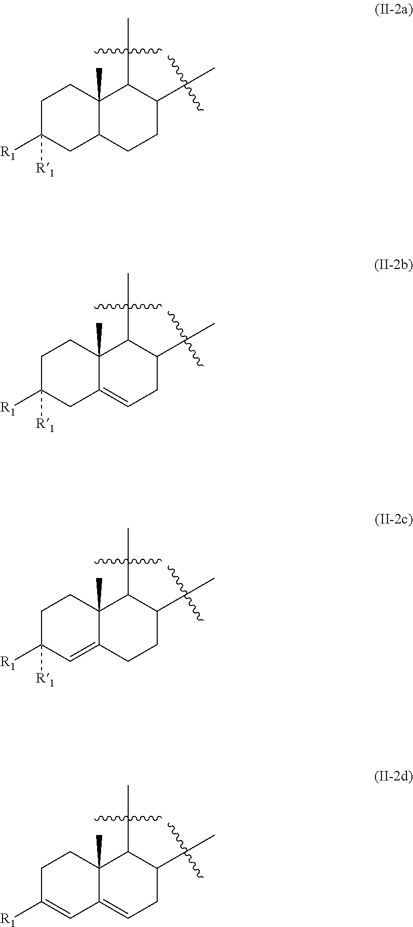

R₁ and R′₁ are each independently selected from H, OR₆, and halogen, or together with the carbon atom to which they are attached form a group C═O, or a 5 to 7 membered heterocyclyl group;

provided that when

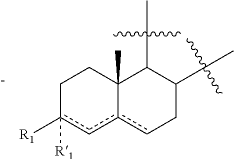

is (II-2d), R₁ cannot be C═O; and
R₆ is H or an alkyl group comprising from 1 to 6 carbon atoms.
   or its pharmaceutically acceptable salts, hydrates or hydrated salts or its polymorphic crystalline structures, racemates, diastereoisomers or enantiomers.

According to a particular embodiment, in formula (II-2), R′₁ is H and R₁ is selected from halogen, in particular F, and OR₆, R₆ being preferably H or Me.

The present invention also relates to a compound of formula (II-3):

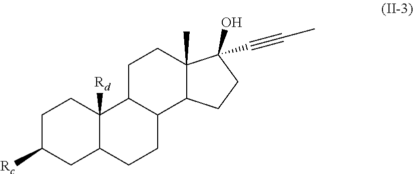

wherein:

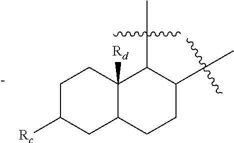

is selected from (II-3a), (II-3b) and (II-3c):

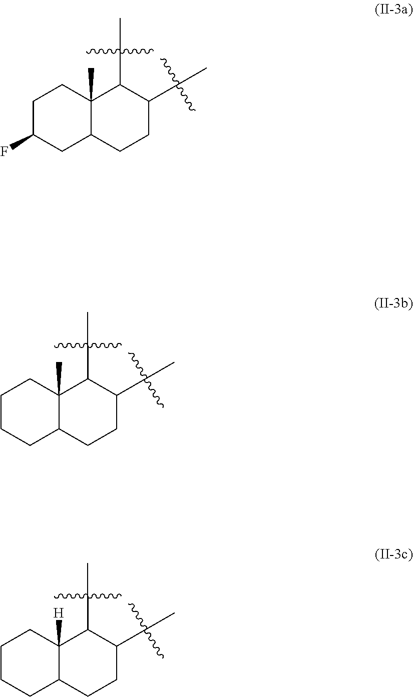 [SMILES: *C1CCC2C[C@@H](F)CC[C@]2(C)C1* ; *C1CCC2CCCC[C@]2(C)C1* ; *C1CCC2CCCC[C@@H]2C1*]

or its pharmaceutically acceptable salts, hydrates or hydrated salts or its polymorphic crystalline structures, racemates, diastereoisomers or enantiomers.

The present invention also relates to a compound of formula (II-2) or (II-3) for its use as a medicament.

The present invention also relates to a medicament comprising a compound of formula (II-2) or (II-3).

The present invention also relates to a pharmaceutical composition comprising a compound of formula (II-2) or (II-3) and a pharmaceutically acceptable excipient.

The present invention also relates to a compound of formula (II-2) or (II-3) for its use for the prevention and/or the treatment of pathologies involving progesterone receptor, in particular for the prevention and/or the treatment of cancer or uterine pathologies.

The present invention also relates to a method for treating or preventing pathologies involving progesterone receptor, in particular for the prevention and/or the treatment of cancer or uterine pathologies, comprising the administration of a pharmaceutically acceptable amount of a compound of formula (II-2) or (II-3) to a patient in need thereof.

The present invention also relates to compounds having one of the following formulae:

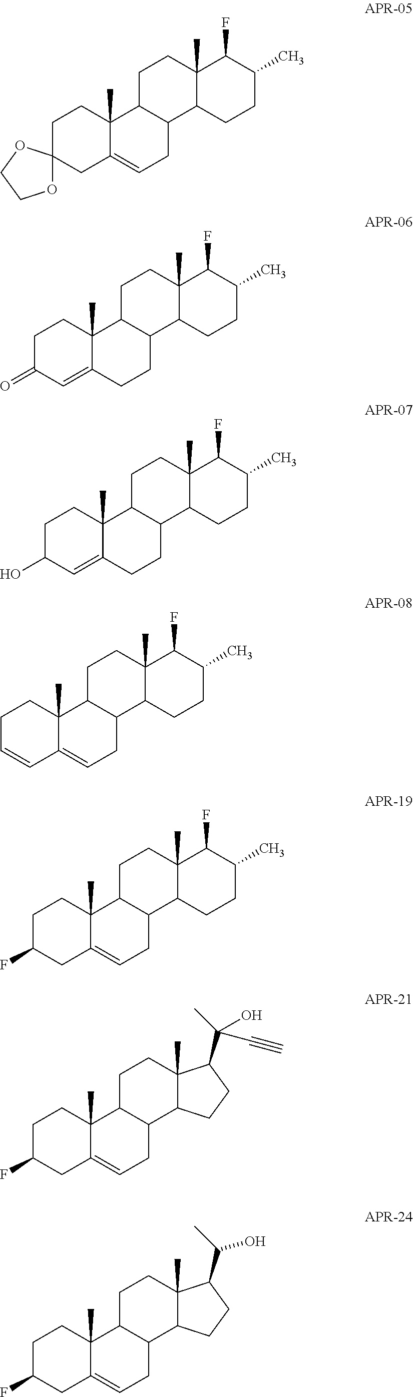

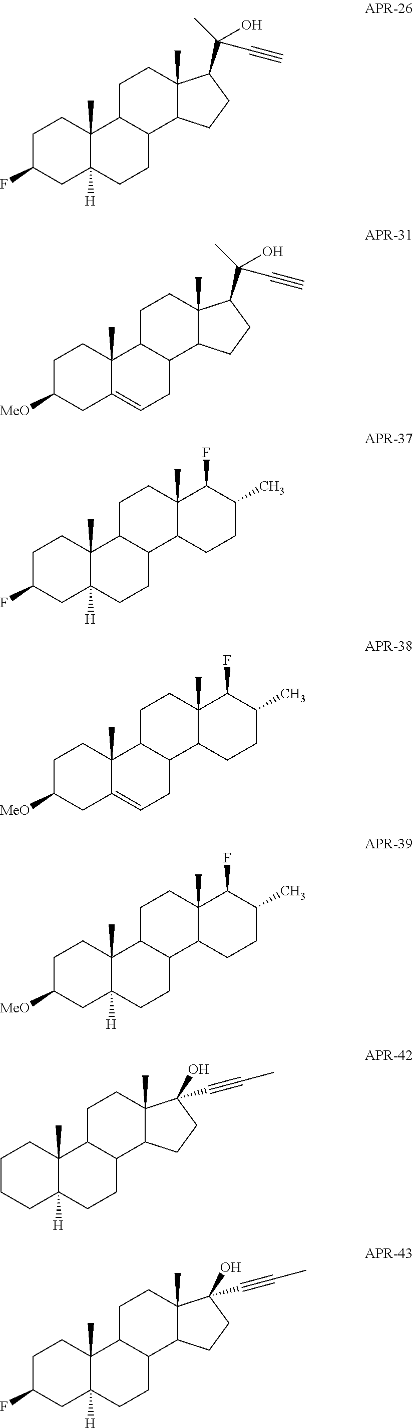

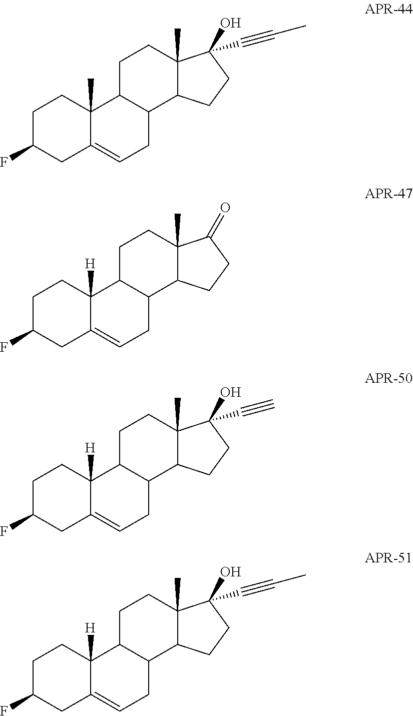 [SMILES: CC#C[C@]1(O)CCC2C3CC=C4C[C@@H](F)CC[C@]4(C)C3CC[C@@]21C ; C[C@]12CCC3C(CC=C4C[C@@H](F)CC[C@@H]43)C1CCC2=O ; C#C[C@]1(O)CCC2C3CC=C4C[C@@H](F)CC[C@@H]4C3CC[C@@]21C ; CC#C[C@]1(O)CCC2C3CC=C4C[C@@H](F)CC[C@@H]4C3CC[C@@]21C]

The present invention also relates to compounds having one of the following formulae:

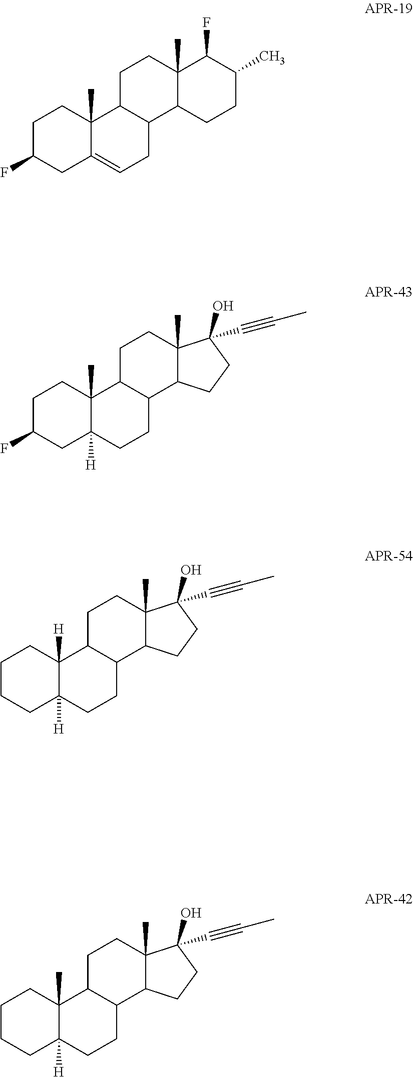 [SMILES: C[C@@H]1CCC2C3CC=C4C[C@@H](F)CC[C@]4(C)C3CC[C@]2(C)[C@H]1F ; CC#C[C@]1(O)CCC2C3CC[C@H]4C[C@@H](F)CC[C@]4(C)C3CC[C@@]21C ; CC#C[C@]1(O)CCC2C3CC[C@H]4CCCC[C@@H]4C3CC[C@@]21C ; CC#C[C@]1(O)CCC2C3CC[C@H]4CCCC[C@]4(C)C3CC[C@@]21C]

The present invention also relates to a compound selected from the above formulae for its use as a medicament.

The present invention also relates to a medicament comprising a compound selected from the above formulae.

The present invention also relates to a pharmaceutical composition comprising a compound selected from the above formulae and a pharmaceutically acceptable excipient.

The present invention also relates to a compound selected from the above formulae for its use for the prevention and/or the treatment of pathologies involving progesterone receptor, in particular for the prevention and/or the treatment of cancer or uterine pathologies.

The present invention also relates to a method for treating or preventing pathologies involving progesterone receptor, in particular for the prevention and/or the treatment of cancer or uterine pathologies, comprising the administration of a pharmaceutically acceptable amount of a compound selected from the above formulae to a patient in need thereof.

The present invention also relates to the compound having formula (II-2) for its use as a drug. The present invention also relates to a pharmaceutical composition comprising at least a compound having formula (II-2) as defined above.

The present invention also relates to a compound of formula (VI):

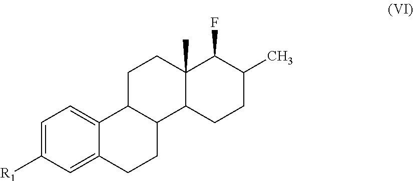

wherein R₁ is H or halogen, in particular F, or its pharmaceutically acceptable salts, hydrates or hydrated salts or its polymorphic crystalline structures, racemates, diastereoisomers or enantiomers.

The present invention also relates to a compound of formula (VII):

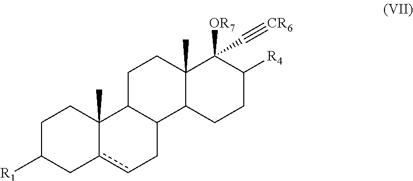

wherein R₁, R₄, R₆ and R₇ are as defined above in formula (I),

R₁ being preferably F, R₄ being preferably methyl, and R₇ being preferably H, or its pharmaceutically acceptable salts, hydrates or hydrated salts or its polymorphic crystalline structures, racemates, diastereoisomers or enantiomers.

The present invention also relates to a compound of formula (VIII):

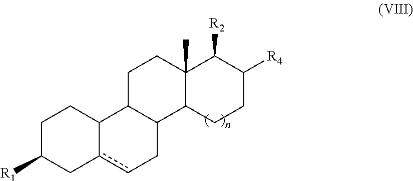

wherein n, R₁ and R₄ are as defined above in formula (I), R₁ being preferably F, R₄ being preferably H or methyl, and

R₂ is an aryl or heteroaryl group, said (hetero)aryl being possibly substituted, or its pharmaceutically acceptable salts, hydrates or hydrated salts or its polymorphic crystalline structures, racemates, diastereoisomers or enantiomers.

The present invention also relates to compounds having formula (VI), (VII) or (VIII) for their use as a drug. The present invention also relates to pharmaceutical compositions comprising at least a compound having formula (VI), (VII) or (VIII) as defined above.

### FIGURES

FIG. 1 shows the efficiency (in %) of APR1, APR12, APR10, APR11, APR13, APR23, APR53, APR14, APR52, APR49, APR32, APR54, APR42, APR54, APR8 in HEK293T cells transiently expressing hPRB. The black column corresponds to the antagonist efficiency and the white column corresponds to the agonist efficiency.

FIG. 2 shows the efficiency (in %) of APR1, APR12, APR10, APR11, APR13, APR23, APR53, APR14, APR52, APR49, APR32, APR54, APR42, APR54, APR8 in MDA-MB-231 iPRAB cells conditionally expressing hPRB. The black column corresponds to the antagonist efficiency and the white column corresponds to the agonist efficiency.

FIG. 3 shows the efficiency (in %) of APR2, APR22, APR27, APR28, APR30, APR31, APR38 and APR39 in HEK293T cells transiently expressing hPRB. The black column corresponds to the antagonist efficiency and the white column corresponds to the agonist efficiency.

FIG. 4 shows the efficiency (in %) of APR2, APR22, APR27, APR28, APR30, APR31, APR38 and APR39 in MDA-MB-231 iPRAB cells conditionally expressing hPRB. The black column corresponds to the antagonist efficiency and the white column corresponds to the agonist efficiency.

FIG. 5 shows the efficiency (in %) of APR15, APR20, APR9, APR18, APR29, APR55, APR48 and APR7 in HEK293T cells transiently expressing hPRB. The black column corresponds to the antagonist efficiency and the white column corresponds to the agonist efficiency.

FIG. 6 shows the efficiency (in %) of APR15, APR20, APR9, APR18, APR29, APR55, APR48 and APR7 in MDA-MB-231 iPRAB cells conditionally expressing hPR B. The black column corresponds to the antagonist efficiency and the white column corresponds to the agonist efficiency.

FIG. 7 shows the efficiency (in %) of APR16, APR17, APR24, APR25, APR21, APR26, APR35, APR33, APR47, APR40, APR41, APR46, APR45, APR36, APR34, APR50, APR43, APR44, APR51, APR19 and APR37 in HEK293T cells transiently expressing hPRB. The black column corresponds to the antagonist efficiency and the white column corresponds to the agonist efficiency.

FIG. 8 shows the efficiency (in %) of APR16, APR17, APR24, APR25, APR21, APR26, APR35, APR33, APR47, APR40, APR41, APR46, APR45, APR36, APR34, APR50, APR43, APR44, APR51, APR19 and APR37 in MDA-MB-231 iPRAB cells conditionally expressing hPRB. The black column corresponds to the antagonist efficiency and the white column corresponds to the agonist efficiency.

FIG. 9 shows the dose-response efficiency (in %) of APR16, APR19, APR43, APR47, APR51 and APR54 in MDA-MB-231 iPRAB cells conditionally expressing hPRB.

FIG. 10 shows the efficiency (in %) of APR16, APR19, APR43, APR47, APR51 and APR54 on PRB-mediated amphiregulin gene transcription. The black column corresponds to the antagonist efficiency and the white column corresponds to the agonist efficiency.

FIG. 11 shows the efficiency (in %) of APR16, APR19, APR43, APR47, APR51 and APR54 in HEK293T cells transiently expressing hAR. The black column corresponds to the antagonist efficiency and the white column corresponds to the agonist efficiency.

FIG. 12 shows the recruitment of the transcriptional co-repressor NcoR by PR upon ligand binding (fold induction as a function of log [ligand]). The black column corresponds to progesterone and the white column corresponds to RU486.

FIG. 13 shows the recruitment of the transcriptional co-repressor SMRT by PR upon ligand binding (fold induction as a function of log [ligand]). The black column corresponds to progesterone and the white column corresponds to RU486.

FIG. 14 shows the recruitment of the transcriptional co-repressors NcoR and SMRT by PR upon RU486 and APRn (APR16, APR19, APR43, APR47, APR51 and APR54) binding. The black column corresponds to NcoR and the white column corresponds to SMRT.

FIG. 15 shows the recruitment of the transcriptional co-activator TIF-2-Nter by PR upon ligand binding (fold induction as a function of log [ligand]). The black column corresponds to progesterone and the white column corresponds to RU486.

FIG. 16 shows the recruitment of the transcriptional co-activator TIF-2-NterRID by PR upon ligand binding (fold induction as a function of log [ligand]). The black column corresponds to progesterone and the white column corresponds to RU486.

FIG. 17 shows the recruitment of the transcriptional co-activator TIF-2 by PR upon progesterone and APRn (APR16, APR19, APR43, APR47, APR51 and APR54) binding. The black column corresponds to TIF2-Nter and the white column corresponds to TIF2-NterRID.

FIG. 18 shows the efficacy of APRn (APR16, APR19, APR43, APR47, APR51 and APR54) to inhibit the progesterone-induced TIF2 recruitment by PR. The black column corresponds to TIF2-Nter and the white column corresponds to TIF2-NterRID.

FIG. 19 shows the efficacy of APR-19 to inhibit the anti-proliferative effects of progesterone on E2-induced endometrial proliferation.

### EXAMPLES

### Chemical Synthesis of Compounds of the Invention

All APRn (antagonist progesterone receptor) compounds of the invention have been obtained by partial synthesis either starting from readily available progesterone, pregnenolone acetate, 17β-hydroxyandrostanolone, (+)-dehydroisoandrosterone or 19-nortestosterone. Derivatization of these steroids at either the carbon-3 and/or carbon-17 was planned in order to examine the relative effect of such selective transformation.

A—Synthesis of Antagonist Progesterone Receptor (APRn) Lacking a C3-Substituent (Compounds Having Formula (I) Wherein R₁═R′₁═H) from Progesterone:

Progesterone was used as starting material for the synthesis of APRn lacking C3-substituent (Scheme 1). Initially, progesterone was reduced with NaBH₄ to give 3β-hydroxysteroid APR-09 ((a) Di Chenna, P. H.; Dansey, V.; Ghini, A. A.; Burton, G. ARKIVOC 2005, 12, 154-162. (b) Mori, M.; Tamaoki, B. Steroids 1977, 29, 517) which upon treatment with H₂ under Pd/C (Diedrich, C. L.; Frey, W.; Christoffers, J. Eur. J. Org. Chem. 2007, 4731) provided 5α steroid APR-10 ((a) Lau, C. K.; Dufresne, C.; Belanger, P. C.; Pietre, S.; Scheigetz, J. J. Org. Chem. 1986, 51, 3038. (b) Kirk, D. N.; Mudd, A. J. Chem. Soc., C 1969, 804), with trans stereochemistry at the A/B ring junction as judged by NMR spectra. This latter was further either transformed quantitatively into the corresponding acetylated APR-13 using acetic anhydride in pyridine or oxidized to provide APR-01.

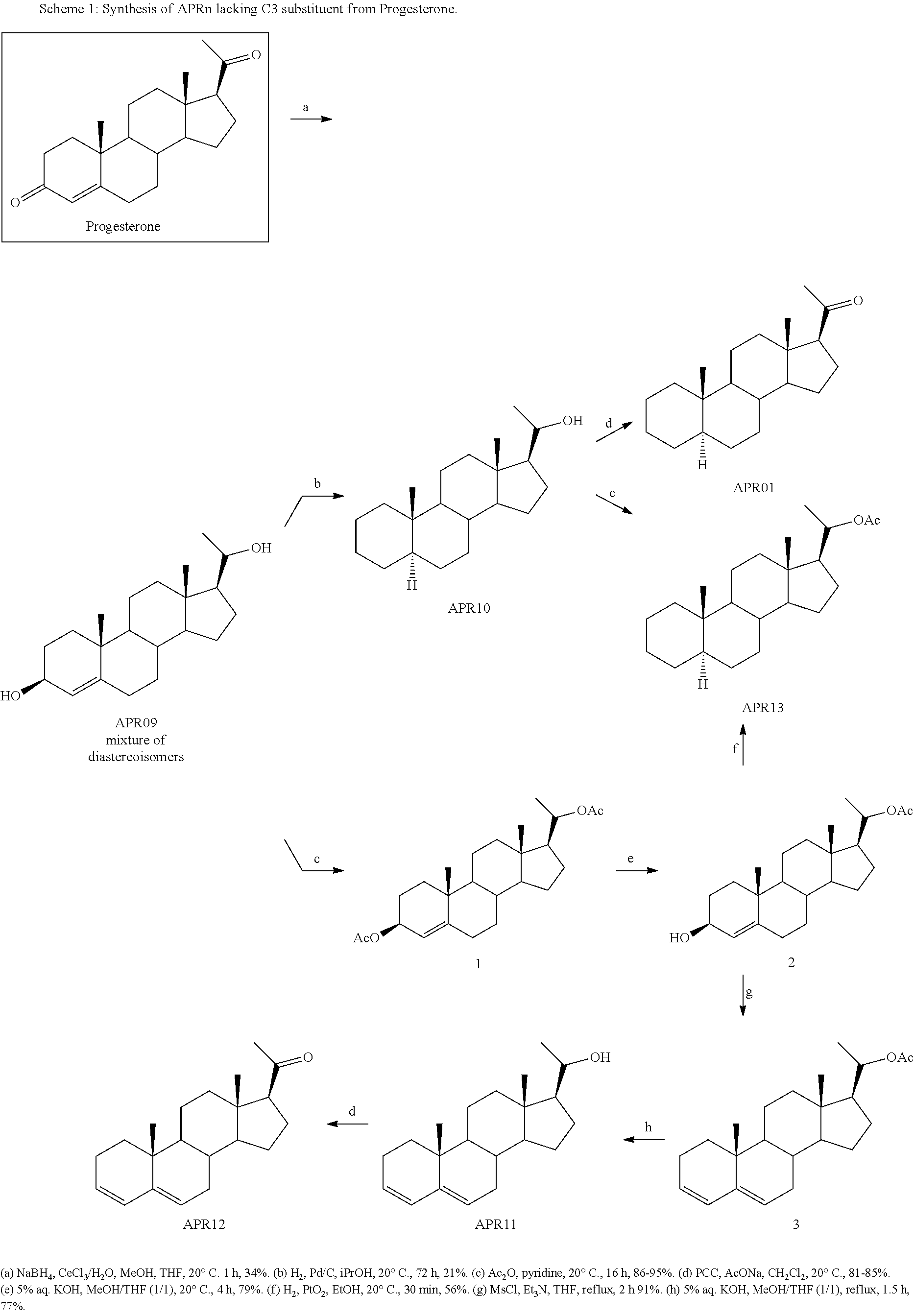

As shown in Scheme 1, APR-12 was synthesized from APR-09 in a five step-sequence. After protection of the alcohol functions, the selective C3-deacetylation of 1 was achieved using 3% of an aqueous solution of potassium hydroxide in MeOH/THF (1/1). Subsequent mesylation (Castellanos, L.; Duque, C.; Rodriguez, J.; Jiménez, C. Tetrahedron 2007, 63, 1544) of 2 under standard conditions directly gave Δ^{3,5} diene steroid 3 which was then transformed into APR-11 by saponification of the acetate function. Further PCC oxidation of the alcohol function yielded APR-12.

### Synthesis of 3β,20-Diacetoxypregn-4-ene (1)

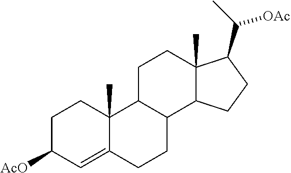 [SMILES: CC(=O)O[C@@H]1C=C2CCC3C(CC[C@@]4(C)C3CC[C@@H]4[C@H](C)OC(C)=O)[C@@]2(C)CC1]

To a solution of progesterone (400 mg, 1.26 mmol) in MeOH (12 mL) and THF (5 mL) was added NaBH₄ (96 mg, 2.52 mmol) and CeCl₃.7H₂O (480 mg, 1.27 mmol) at room temperature. After 1 h, the mixture was quenched with ethyl acetate and was washed with 10% HCl. The organic layer was dried over MgSO₄ and concentrated. Without purification, the crude 3β,20-dihydroxy-4-pregnene was dissolved in acetic anhydride (3 mL) and pyridine (2 mL) was added DMAP (10 mg, 82 μmol), the solution was stirred for 16 h at room temperature, then diluted with dichloromethane and washed with 5% HCl, 5% NaHCO₃ and finally with water, the organic layer was dried with Na₂SO₄, filtered and the solvent evaporated. The crude product was purified by chromatography on silica gel (eluant: cyclohexane/EtOAc, 90/10) to afford (1) (437 mg, 86% yield) as a white solid. R_{f}=0.74 (eluant: cyclohexane/EtOAc, 60/40). IR (v cm⁻¹): 854, 1025, 1074, 1239, 1369, 1449, 1728, 2930. ¹H NMR (CDCl₃, 300 MHz): δ 0.57 (s, 3H, Me-18), 0.98 (s, 3H, Me-19), 1.06 (d, 3H, J=6.1 Hz, Me-21), 1.93 (s, 3H, OAc), 1.96 (s, 3H, OAc), 0.54-2.18 (m, 20H), 4.75 (m, 1H, H-20), 5.11-5.14 (m, 2H, H-3, H-4). From the ¹³C NMR data this product was determined to be 9:1 mixture of epimers at C₂₀, the ¹³C NMR (CDCl₃, 75 MHz) for the major β-isomer were δ 12.4, 18.8, 19.9, 20.8, 21.3, 21.4, 24.2, 25.0, 25.4, 32.1, 32.9, 35.0, 35.7, 37.3, 39.1, 42.2, 54.2, 54.9, 55.4, 70.8, 72.7, 119.1, 149.2, 170.2, 170.8. MS (APCI+) m/z 425.0 (M+Na)⁺.

### Synthesis of 20-Acetoxypregn-4-en-3β-ol (2)

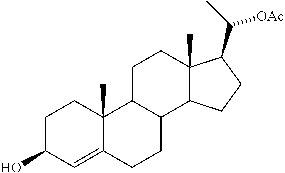 [SMILES: CC(=O)O[C@@H](C)[C@H]1CCC2C3CCC4=C[C@@H](O)CC[C@]4(C)C3CC[C@@]21C]

The 3β,20-diacetoxypregn-4-ene (1) (1 g, 2.48 mmol) was dissolved in THF (30 mL) and methanol (30 mL). To this solution was added 5% aqueous KOH (2.85 mL) and the mixture was stirred for 3 h at room temperature, concentrated to ⅓ of its volume, diluted with water and extracted with dichloromethane. The organic layer was dried over MgSO₄, filtered and evaporated. The amorphous solid was purified by flash chromatography (eluant: cyclohexane/EtOAc, 80/20) to give (2) (710 mg, 79%) as a white solid. R_{f}=0.31 (eluant: cyclohexane/EtOAc, 80/20). IR (v cm⁻¹): 1027, 1246, 1370, 1449, 1719, 2930, 3330. ¹H NMR (CDCl₃, 300 MHz): δ 0.65 (s, 3H, Me-18), 1.04 (s, 3H, Me-19), 1.14 (d, 3H, J=6.1 Hz, Me-21), 2.01 (s, 3H, OAc), 0.58-2.09 (m, 19H), 2.14-2.25 (m, 1H), 4.14 (m, 1H, H-3), 4.83 (m, 1H, H-20), 5.28 (m, 1H, H-4). From the ¹³C NMR data this product was determined to be 9:1 mixture of epimers at C₂₀, the ¹³C NMR (CDCl₃, 75 MHz) for the major β-isomer were δ 12.6, 19.0, 20.0, 21.1, 21.7, 24.4, 25.6, 29.6, 32.3, 33.2, 35.5, 35.9, 37.5, 39.4, 42.4, 54.6, 55.1, 55.7, 68.1, 73.0, 123.6, 147.6, 170.6. MS (APCI+) m/z 383.0 (M+Na)⁺.

### Synthesis of 20-Acetoxypregn-3,5-diene (3)

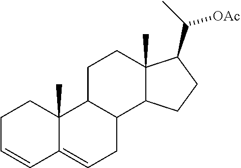 [SMILES: CC(=O)O[C@@H](C)[C@H]1CCC2C3CC=C4C=CCC[C@]4(C)C3CC[C@@]21C]

A solution of 20-acetoxypregn-4-en-3-ol (2) (500 mg, 1.38 mmol) in THF (15 mL) was cooled to 0° C. and methanesulfonyl chloride (0.16 mL, 2.08 mmol) was added dropwise. The mixture was refluxed for 2 h and then allowed to reach room to temperature. The water was added and then the aqueous layer was extracted with CH₂Cl₂. The organic layer was dried over MgSO₄ and concentrated. The crude product was purified by chromatography on silica gel (eluant: cyclohexane/EtOAc, 95/05) to afford (3) (434 mg, 91% yield) as a white solid. R_{f}=0.72 (eluant: cyclohexane/EtOAc, 80/20). IR (v cm⁻¹): 850, 962, 1020, 1242, 1373, 1724, 2935. From the ¹H NMR and ¹³C NMR data this product was determined to be 9:1 mixture of epimers at C₂₀, the ¹H NMR (CDCl₃, 300 MHz) for the major β-isomer were δ 0.67 (s, 3H, Me-18), 0.94 (s, 3H, Me-19), 1.15 (d, 3H, J=6.1 Hz, Me-21), 2.02 (s, 3H, OAc), 0.85-2.25 (m, 18H), 4.85 (m, 1H, H-20), 5.38 (m, 1H, H-6), 5.58 (m, 1H, H-3), 5.92 (d, 1H, J=9.7 Hz, H-4). The ¹³C NMR (CDCl₃, 75 MHz) for the major β-isomer were δ 12.6, 18.9, 20.0, 21.0, 21.6, 23.2, 24.3, 25.6, 31.8, 31.9, 33.9, 35.3, 39.3, 42.4, 48.6, 55.1, 56.5, 73.0, 123.0, 125.1, 129.1, 141.6, 170.5. MS (APCI+) m/z 343.0 (M+H)⁺.

### Example 1

### Synthesis of pregn-4-en-3β,20-diol (APR-09)

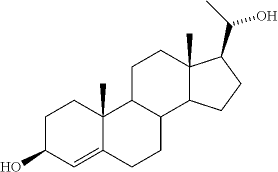 [SMILES: C[C@H](O)[C@H]1CCC2C3CCC4=C[C@@H](O)CC[C@]4(C)C3CC[C@@]21C]

To a solution of progesterone (1 g, 3.18 mmol) in MeOH (30 mL) and THF (12 mL) was added NaBH₄ (240 mg, 6.36 mmol) and CeCl₃.7H₂O (1.2 g, 3.18 mmol) at room temperature. The mixture was stirred for 1 h. The excess amount of NaBH₄ was quenched with ethyl acetate and the mixture was washed with 10% HCl. The organic layer was dried over MgSO₄ and concentrated. The crude product was purified by chromatography on silica gel (eluant: cyclohexane/EtOAc, 60/40) to afford (APR-09) (343 mg, 34% yield) as a white solid. R_{f}=0.37 (eluant: cyclohexane/EtOAc, 50/50). Mp 169-170° C. IR (v cm⁻¹): 879, 962, 1028, 1375, 1448, 2921, 3332. ¹H NMR (CDCl₃, 300 MHz): δ 0.76 (s, 3H, Me-18), 1.04 (s, 3H, Me-19), 1.12 (d, 3H, J=6.1 Hz, Me-21), 0.67-1.76 (m, 16H), 1.90-2.08 (m, 3H), 2.13-2.25 (m, 1H), 3.71 (m, 1H, H-20), 4.13 (m, 1H, H-3α), 5.27 (m, 1H, H-4). ¹³C NMR (Acetone-d₆, 75 MHz): δ 13.6, 20.3, 22.6, 25.2, 26.2, 27.4, 31.2, 33.9, 35.2, 37.6, 37.7, 39.0, 41.6, 44.3, 56.8, 57.7, 60.2, 68.7, 71.1, 127.0, 147.1. MS (APCI+) m/z 341.0 (M+Na)⁺.

### Example 2

### Synthesis of pregn-3,5-dien-20-ol (APR-11)

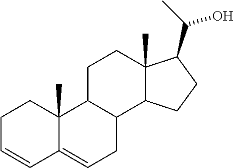 [SMILES: C[C@H](O)[C@H]1CCC2C3CC=C4C=CCC[C@]4(C)C3CC[C@@]21C]

The same procedure for the synthesis of (2) was followed. From (3), the compound (APR-11) was obtained after chromatography on silica gel (eluant: petroleum ether/EtOAc, 98/02) in 77% yield. IR (v cm⁻¹): 967, 1094, 1373, 2176, 2928, 3354. Pregn-3,5-dien-20β-ol: Rf=0.43 (eluant: petroleum ether/EtOAc, 8/2). White solid. Mp 119-120° C. ¹H NMR (CDCl₃, 300 MHz): δ 0.80 (s, 3H, Me-18), 0.96 (s, 3H, Me-19), 1.15 (d, 3H, J=6.1 Hz, Me-21), 0.82-2.21 (m, 18H), 3.75 (m, 1H, H-20), 5.38 (m, 1H, H-6), 5.59 (m, 1H, H-3), 5.93 (d, 1H, J=9.7 Hz, H-4). ¹³C NMR (CDCl₃, 75 MHz) for the major β-isomer were δ 12.7, 18.9, 21.0, 23.2, 23.8, 24.6, 25.8, 31.8, 31.9, 33.9, 35.4, 40.1, 42.5, 48.5, 56.6, 58.7, 70.7, 123.1, 125.2, 129.1, 141.7. MS (APCI+) m/z 301.0 (M+H)⁺. Pregn-3,5-dien-20α-ol: Rf=0.28 (eluant: petroleum ether/EtOAc, 8/2). White solid. Mp 103-104° C. ¹H NMR (CDCl₃, 300 MHz): δ 0.70 (s, 3H, Me-18), 0.95 (s, 3H, Me-19), 1.24 (d, 3H, J=6.3 Hz, Me-21), 0.85-2.20 (m, 18H), 3.74 (m, 1H, H-20), 5.39 (m, 1H, H-6), 5.59 (m, 1H, H-3), 5.92 (d, 1H, J=9.6 Hz, H-4). ¹³C NMR (CDCl₃, 75 MHz) for the minor α-isomer were δ 12.7, 18.9, 20.8, 23.2, 23.7, 24.3, 25.9, 31.6, 31.9, 33.9, 35.4, 39.0, 41.9, 48.5, 56.9, 58.6, 70.5, 123.1, 125.2, 129.1, 141.6. MS (APCI+) m/z 301.0 (M+H)⁺.

### Example 3

### Synthesis of Pregn-3,5-dien-20-one (APR-12)

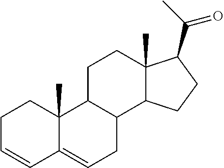 [SMILES: CC(=O)[C@H]1CCC2C3CC=C4C=CCC[C@]4(C)C3CC[C@@]21C]

A suspension of pyridinium chlorochromate (323 mg, 1.50 mmol), sodium acetate (68 mg, 0.83 mmol) and 3 Å molecular sieves in anhydrous dichloromethane (4 mL) was stirred for 5 min under a nitrogen atmosphere. A solution of the (APR-11) (100 mg, 0.33 mmol) in anhydrous dichloromethane (4 mL) was added and stirring continued at room temperature for 2 h. The mixture was filtered through celite and the solvent was concentrated. The crude product was chromatographed on silica gel (eluant: cyclohexane/EtOAc, 90:10) to afford (APR-12) (80 mg, 81% yield) as a white solid. R_{f}=0.2 (eluant: cyclohexane/EtOAc, 50/50). Mp 135-136° C. IR (v cm⁻¹): 841, 1153, 1353, 1701, 2926. ¹H NMR (CDCl₃, 300 MHz): δ 0.66 (s, 3H, Me-18), 0.95 (s, 3H, Me-19), 2.13 (s, 3H, Me-21), 0.74-2.24 (m, 17H), 2.54 (m, 1H, H-17), 5.39 (m, 1H, H-6), 5.60 (m, 1H, H-3), 5.93 (d, 1H, J=9.7 Hz, H-4). ¹³C NMR (CDCl₃, 75 MHz): δ 13.5, 18.9, 21.1, 23.0, 23.2, 24.5, 31.7, 31.8, 31.9, 33.9, 35.4, 39.0, 44.3, 48.4, 57.3, 63.9, 122.9, 125.3, 129.0, 141.6, 209.7. MS (APCI+) m/z 321.0 (M+Na)⁺.

### Example 4

### Synthesis of 20-Acetoxy-5α-pregnane (APR-13)

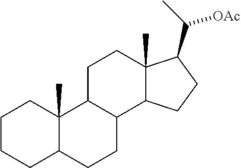 [SMILES: CC(=O)O[C@@H](C)[C@H]1CCC2C3CCC4CCCC[C@]4(C)C3CC[C@@]21C]

To a solution of (2) (500 mg, 1.38 mmol) in ethanol (5 mL) was added PtO₂ catalyst (113 mg, 20%) and hydrogenation was carried at room temperature in atmospheric pressure for 12 h. The reaction mixture was filtered and the filtrate was evaporated under reduced pressure. The crude product was chromatographed on silica gel (eluant: cyclohexane/EtOAc, 98/02) to afford (APR-13) (269 mg, 56% yield) as a white solid. R_{f}−0.76 (eluant: cyclohexane/EtOAc, 80/20). IR (v cm⁻¹): 1019, 1241, 1371, 1442, 1728, 2915. From the ¹H NMR and ¹³C NMR data this product was determined to be 24:76 mixture of epimers at C₂₀, the ¹H NMR (CDCl₃, 300 MHz) for the major β-isomer were δ 0.60 (s, 3H, Me-18), 0.76 (s, 3H, Me-19), 1.13 (d, 3H, J=6.1 Hz, Me-21), 0.69-1.89 (m, 25H), 1.99 (s, 3H, OAc), 4.82 (m, 1H, H-20). The ¹³C NMR (CDCl₃, 75 MHz) for the major β-isomer were δ 12.4, 12.7, 20.0, 20.9, 21.6, 22.3, 24.3, 25.6, 25.7, 27.0, 29.2, 32.3, 35.5, 38.8, 39.6, 47.2, 55.0, 55.3, 56.2, 73.0, 170.5. MS (APCI+) m/z 347.0 (M+H)⁺.

### Example 5

### Synthesis of 20-Hydroxypregnane (APR-10)

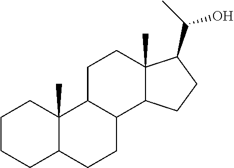 [SMILES: C[C@H](O)[C@H]1CCC2C3CCC4CCCC[C@]4(C)C3CC[C@@]21C]

To a solution of (APR-09) (162 mg, 0.51 mmol) in isopropanol (4 mL) was added Pd/C (30 mg, 10%) and hydrogenation was carried at room temperature under atmospheric pressure for 72 h. The reaction mixture was filtered and the filtrate was evaporated under reduced pressure. The crude product was purified on a silica gel column chromatography (eluant: cyclohexane/EtOAc, 7/3) to afford (APR-10) in 21% yield. R_{f}=0.77 (Cyclohexane/Acétate d'éthyle: 7/3). IR (v cm⁻¹): 3380, 2922, 2860, 1448, 1375, 1087, 1011, 966, 878. RMN ¹H δ (300 MHz) ppm: 0.79 (s, 3H, CH₃), 0.89 (s, 3H, CH₃), 1.05 (d, 3H, CH₃, J=6.0 Hz), 0.50-2.10 (m, 34H), 3.65 (dq, 1H, J=6.1 Hz, J=10.0 Hz). RMN ¹³C (75 MHz) δ ppm: 70.6, 58.7, 58.6, 56.1, 54.7, 47.1, 43.7, 42.6, 42.5, 40.6, 40.4, 40.3, 38.7, 37.6, 36.3, 35.7, 35.6, 35.4, 32.2, 29.1, 25.7, 25.6, 24.5, 24.4, 24.3, 23.5, 22.2, 21.3, 20.7, 12.6. MS (ESI): m/z=327.3 [M+Na]⁺

### Example 6

### 5α-pregnan-20-one (APR-01)

This compound is a commercial product obtained from Steraloids (Newport, R.I. USA).

B—Synthesis of Antagonist Progesterone Receptor (APRn) Lacking a C3-Substituent (Compounds Having Formula (I) Wherein R₁═R′₁═H) from 17β-Hydroxy Androstanolone:

For the synthesis of 17-ethynyl APRn analogues with no substituent at the A-ring, 17-hydroxy androstanolone has been used as starting material (Scheme 2). Thus, selective transformation of the 3-keto function into the methylene group was achieved via a clemmensen-type reduction using zinc dust in acetic acid to produce APR-14 (Salvador, J. A. R.; Sá e Melo, M. L.; Neves, A. S. C. Tetrahedron Lett. 1993, 34, 361-362). PCC alcohol oxidation furnished APR-23 (Makoto, O.; Kozaburo, N. Synthesis 1994, 6, 624-628) which was then reacted with metal acetylide to produce 17a-alkynyl APR-32 and APR-42 ((a) Djerassi, C.; Yashin, R.; Rosenkranz, G. J. Am. Chem. Soc. 1950, 72, 5750-5751. (b) Fernandez, C.; Diouf, O.; Moman, E.; Gomez, G.; Fall, Y. Synthesis 2005, 1701-1705. (c) Hungerford, N. L.; McKinney, A. R.; Stenhouse, A. M.; McLeod, M. D. Org. Biomol. Chem. 2006, 4, 3951-3959).

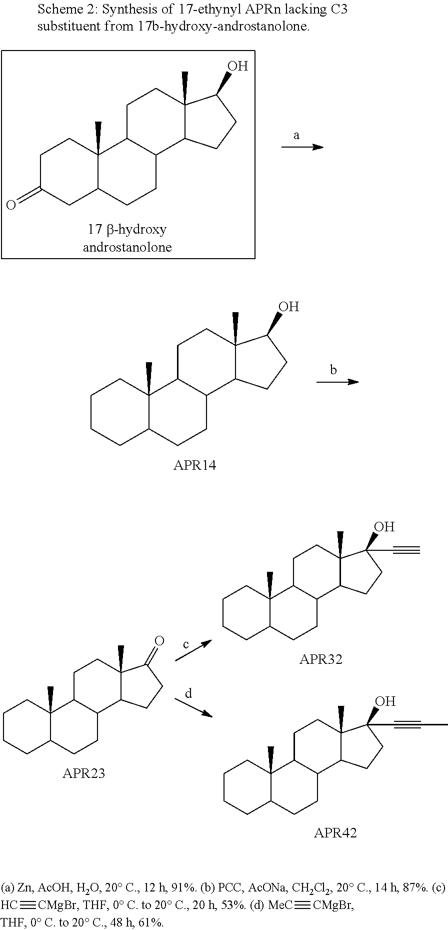

### Example 7

### Synthesis of 5α-Androstan-17β-ol (APR-14)

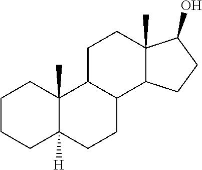 [SMILES: C[C@]12CCC3C(CC[C@H]4CCCC[C@]34C)C1CC[C@@H]2O]

To a solution of 17β-hydroxy-5α-androstan-3-one (1 g, 3.44 mmol) in acetic acid (20 mL) and H₂O (10 mL) was added Zn (10 g, 154 mmol, 5 μm, Aldrich), the solution was stirred for 12 h at room temperature, then filtred through celite and the filtrate neutralized with NaHCO₃. The aqueous layer was extracted with CH₂Cl₂. The organic layer was dried with Na₂SO₄, filtered and the solvent was evaporated. The crude product was chromatographed on silica gel (eluant: cyclohexane/EtOAc, 90:10) to afford (APR-14) (862 mg, 91% yield) as a white solid. Mp 170-171° C. IR (v cm⁻¹): 1053, 1967, 2145, 2921, 3288. ¹H NMR (CDCl₃, 300 MHz): δ 0.73 (s, 3H, Me-18), 0.79 (s, 3H, Me-19), 0.61-1.68 (m, 22H), 1.78 (m, 1H, H-15), 2.04 (m, 1H, H-16), 3.62 (m, 1H, H-17). ¹³C NMR (CDCl₃, 75 MHz): δ 11.3, 12.4, 20.6, 22.3, 23.5, 27.0, 29.1, 29.2, 30.7, 31.9, 35.8, 36.5, 37.0, 38.9, 43.2, 47.3, 51.3, 55.1, 82.2. MS (ESI+) m/z 299.0 (M+Na)⁺.

### Example 8

### Synthesis of 5α-Androstan-17-one (APR-23)

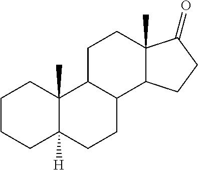 [SMILES: C[C@]12CCC3C(CC[C@H]4CCCC[C@]34C)C1CCC2=O]

The same procedure for the synthesis of (APR-12) was followed. From (APR-14), the compound (APR-23) was obtained after chromatography on silica gel (eluant: cyclohexane/EtOAc, 90/10) in 87% yield as a white solid. R_{f}−0.53 (eluant: cyclohexane/EtOAc, 80/20). Mp 120-121° C. IR (v cm⁻¹): 1010, 1376, 1448, 1742, 2852, 2919. ¹H NMR (CDCl₃, 300 MHz): δ 0.80 (s, 3H, Me-18), 0.85 (s, 3H, Me-19), 0.68-2.11 (m, 23H), 2.42 (m, 1H, H-16). ¹³C NMR (CDCl₃, 75 MHz): δ 12.4, 14.0, 20.2, 21.9, 22.3, 26.9, 28.9, 29.2, 31.2, 31.8, 35.3, 36.0, 36.6, 38.8, 47.2, 48.0, 51.8, 55.0, 221.6. MS (ESI+) m/z 297.0 (M+Na)⁺.

### Example 9

### Synthesis of 17α-Ethynyl-5α-androstan-17β-ol (APR-32)

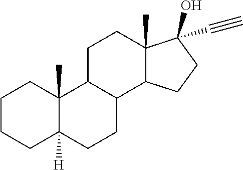 [SMILES: C#C[C@]1(O)CCC2C3CC[C@H]4CCCC[C@]4(C)C3CC[C@@]21C]

The same procedure for the synthesis of (APR-21) was followed. From (APR-23), the compound (APR-32) was obtained after chromatography on silica gel (eluant: cyclohexane/EtOAc, 98/02) in 53% yield as a white solid. R_{f}=0.52 (eluant: cyclohexane/EtOAc, 80/20). Mp 152-153° C. (lit. 144-150° C.). IR (v cm⁻¹): 1013, 1454, 2160, 2849, 2923, 3315. ¹H NMR (CDCl₃, 300 MHz): δ 0.79 (s, 3H, Me-18), 0.83 (s, 3H, Me-19), 0.66-1.84 (m, 22H), 1.96 (m, 1H, H-15), 2.27 (m, 1H, H-16), 2.56 (s, 1H, H_{C≡C}). ¹³C NMR (CDCl₃, 75 MHz): δ 12.4, 13.0, 20.6, 22.3, 23.3, 27.0, 29.1, 29.2, 31.9, 33.0, 36.3, 36.5, 38.9, 39.1, 47.1, 47.2, 50.8, 54.6, 73.9, 80.2. MS (ESI+) m/z 323.0 (M+Na)⁺.

### Example 10

### Synthesis of 17α-(1-Propynyl)-5α-androstan-17β-ol (APR-42)

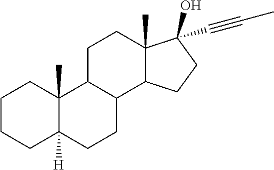 [SMILES: CC#C[C@]1(O)CCC2C3CC[C@H]4CCCC[C@]4(C)C3CC[C@@]21C]

To a solution of (APR-23) (100 mg, 364 μmol) in dry THF (2 mL) was added 1-propynylmagnesium bromide (14.57 mL, 7.28 mmol, 0.5M in THF) at 0° C. The solution was stirred at room temperature under nitrogen atmosphere 48 h. Then, saturated aq NH₄Cl was added and the mixture was thoroughly extracted with EtOAc. The solvent was removed under reduced pressure and subsequent purification by flash chromatography (eluant: cyclohexane/EtOAc, 95/05) to afford (APR-42) (70 mg, 61% yield) as a white solid. R_{f}=0.50 (eluant: cyclohexane/EtOAc, 80/20). Mp 150-151° C. IR (v cm⁻¹): 852, 986, 1017, 1248, 1378, 1449, 2857, 2920, 3523. ¹H NMR (CDCl₃, 300 MHz): δ 0.71 (m, 1H), 0.79 (s, 3H, Me-18), 0.81 (s, 3H, Me-19), 0.85-1.74 (m, 22H), 1.88 (s, 3H, Me), 1.91-1.97 (m, 1H), 2.19 (m, 1H). ¹³C NMR (CDCl₃, 75 MHz): δ 3.9, 12.4, 13.1, 20.7, 22.4, 23.3, 27.0, 29.1, 29.2, 31.9, 33.1, 36.4, 36.5, 38.9, 39.2, 47.1, 47.3, 50.7, 54.6, 80.4, 81.7, 83.1. MS (ESI+) m/z 337.0 (M+Na)⁺.

C—Synthesis of Antagonists Progesterone Receptor (APRn) Bearing a C3 Fluorine Atom (Compounds of Formula (I) Wherein R₁═F and R′₁═H) from Pregnenolone Acetate:

Readily available pregnenolone acetate was next used as starting material for the synthesis of APRn analogues having at the C3 position a fluorine atom (Scheme 3).

APR-18 was obtained from pregnenolone acetate in a two-step sequence by reduction of the 20-keto group followed by saponification of the acetate function of 4. The reduction of D5 double bond of 4 was achieved using H₂ and Pd/C in AcOEt to form 5a steroid APR-29, with trans stereochemistry at the A/B ring junction. When APR-18 was submitted to diethylaminosulfurtrifluoride (DAST) in CH₂Cl₂, a simultaneous fluorination of the alcohol function together with a D-ring-expansion ((a) Nishizawa, M.; Iwamoto, Y.; Takao, H.; Imagawa, H.; Sugihara, T. Org. Lett. 2000, 2, 1685-1687. (b) Nishizawa, M.; Asai, Y.; Imagawa, H. Org. Lett. 2006, 8, 5793-5796) occurred, furnishing exclusively difluorinated homosteroid APR-19 in 69% yield. In addition to the MS and NMR spectra, the X-ray crystallography pattern of APR-19 clearly indicated the a-stereochemistry of 17-methyl group as well as the b-stereochemistry of the 3- and 17α-fluoro atoms substituents.

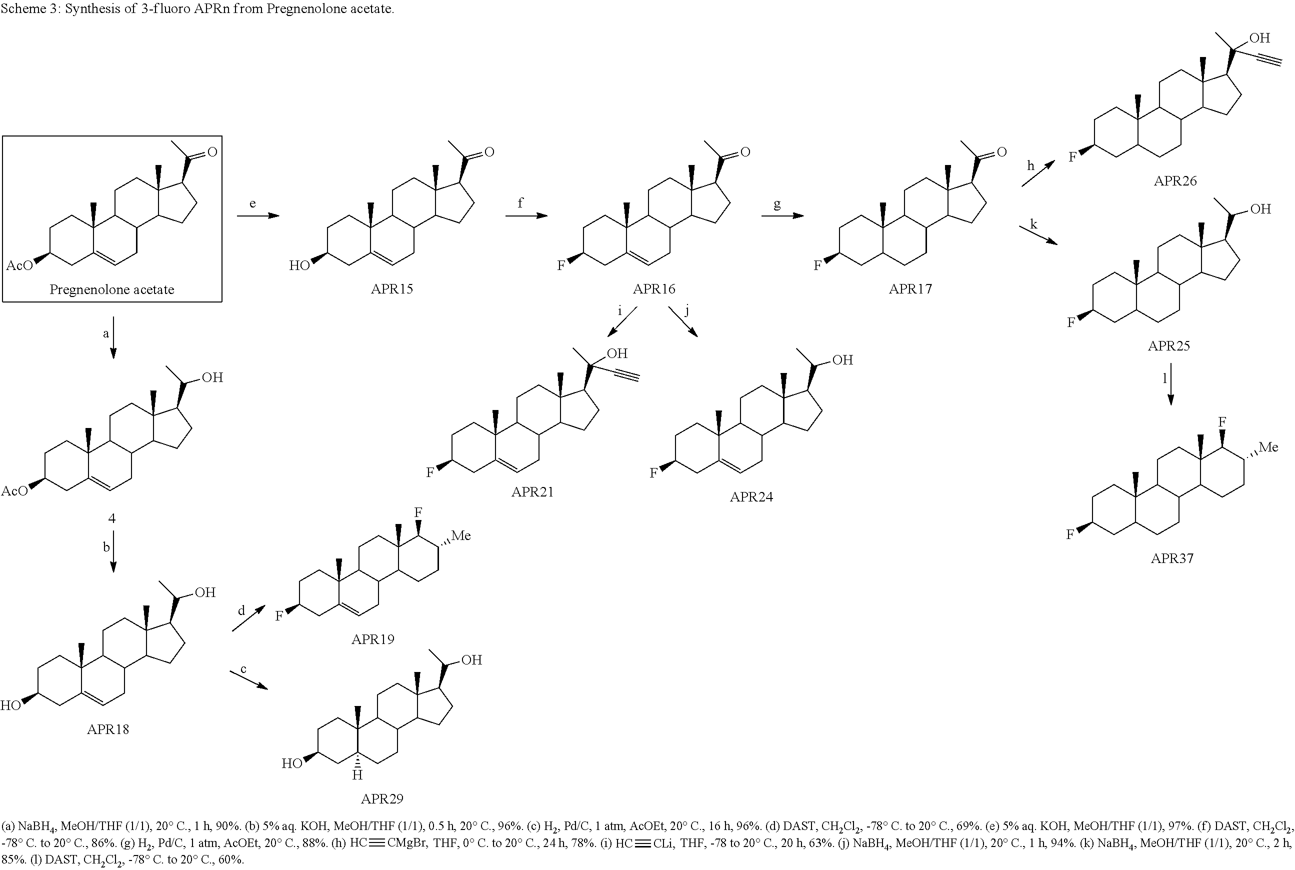

Deacetylation reaction of pregnenolone acetate under alkaline conditions furnished pregnenolone APR-15. Subsequent fluorination with DAST, a highly effective nucleophilic fluorinating agent, in CH₂Cl₂ successfully provided 3-fluoro derivative APR-16. In addition to the MS and NMR spectra, the X-ray crystallography pattern of APR-16 clearly indicated the pstereochemistry of the 3-fluoro atom substituent.

The catalytic hydrogenation of Δ⁵ double bond in APR-16 using Pd/C gave the 5a steroid APR-17, with trans stereochemistry at the A/B ring junction (Scheme 3)(Monsalve, L. N.; Machado Rada, M. Y.; Ghini, A. A.; Baldessari, A. Tetrahedron 2008, 64, 1721-1730). With the synthesized APR-16 and APR-17 was carried out a reaction with metal acetylide. It has been established the arising acetylene alcohols reaction with metal acetylide. It has been established the arising acetylene alcohols APR-21 and APR-26 contained two epimers at C20 atom which were virtually indistinguishable both by chromatography and ¹H NMR spectra. The formation of two epimers was detected only with the use of ¹³C NMR spectroscopy.

The 20-keto functions of APR-16 and APR-17 were also reduced using NaBH₄ in MeOH/THF (1/1) to produce quantitatively APR-24 and APR-25, respectively as a mixture of two epimers at C20 in a 14:86 C20α/C20β ratio. Finally, treatment of APR-25 with DAST furnished, as expected, the rearrangement product difluorinated APR-37 as a single isomer in 60% yield (Scheme 3).

### Synthesis of 3β-Acetoxypregn-5-en-20-ol (4)

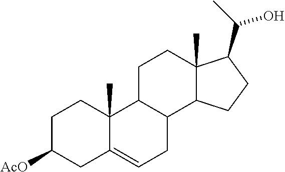 [SMILES: CC(=O)O[C@H]1CC[C@@]2(C)C(=CCC3C2CC[C@@]2(C)C3CC[C@@H]2[C@H](C)O)C1]

The same procedure for the synthesis of (APR-09) was followed. From pregnenolone acetate, the compound (4) was obtained after chromatography on silica gel (eluant: petroleum ether/EtOAc, 80/20) in 90% yield as a mixture (3:7, α/β). IR (v cm⁻¹): 883, 1031, 1254, 1367, 1721, 2937, 3557. MS (APCI+) m/z 383.0 (M+Na)⁺. 3β-Acetoxypregn-5-en-20β-ol: Rf=0.31 (eluant: petroleum ether/EtOAc, 8/2). White solid. Mp 164-165° C. ¹H NMR (CDCl₃, 300 MHz): δ 0.77 (s, 3H, Me-18), 1.03 (s, 3H, Me-19), 1.14 (d, 3H, J=6.0 Hz, Me-21), 2.03 (s, 3H, OAc), 0.83-2.11 (m, 19H), 2.32 (m, 2H), 3.74 (m, 1H, H-20), 4.60 (m, 1H, H-3), 5.37 (m, 1H, H-6). ¹³C NMR (CDCl₃, 75 MHz) for the major β-isomer were δ 12.5, 19.5, 21.0, 21.6, 23.9, 24.7, 25.8, 27.9, 31.8, 32.0, 36.8, 37.1, 38.3, 40.0, 42.4, 50.2, 56.3, 58.7, 70.7, 74.1, 122.6, 139.9, 170.7. MS (ESI+) m/z 383.0 (M+Na)⁺. 3β-Acetoxypregn-5-en-20α-ol: Rf=0.22 (eluant: petroleum ether/EtOAc, 8/2). White solid. Mp 123-124° C. ¹H NMR (CDCl₃, 300 MHz): δ 0.68 (s, 3H, Me-18), 1.02 (s, 3H, Me-19), 1.24 (d, 3H, J=6.1 Hz, Me-21), 0.79-1.97 (m, 19H), 2.03 (s, 3H, OAc), 2.32 (m, 2H), 3.73 (m, 1H, H-20), 4.60 (m, 1H, H-3), 5.38 (m, 1H, H-6). ¹³C NMR (CDCl₃, 75 MHz) for the minor α-isomer were δ 12.6, 19.4, 20.9, 21.6, 23.7, 24.3, 25.8, 27.9, 31.7, 32.0, 36.8, 37.1, 38.2, 38.9, 41.7, 50.1, 56.6, 58.6, 70.4, 74.1, 122.6, 139.8, 170.7. MS (ESI+) m/z 383.0 (M+Na)⁺.

### Example 11

### Synthesis of 3β-Hydroxypregn-5-en-20-one (APR-15)

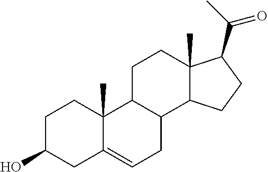 [SMILES: CC(=O)[C@H]1CCC2C3CC=C4C[C@@H](O)CC[C@]4(C)C3CC[C@@]21C]

The same procedure for the synthesis of (2) was followed. From pregnenolone acetate, the compound (APR-15) was obtained after chromatography on silica gel (eluant: cyclohexane/EtOAc, 98/02) in 97% yield as a white solid. R_{f}=0.56 (eluant: cyclohexane/EtOAc, 80/20). Mp 190-191° C. IR (v cm⁻¹): 952, 1050, 1194, 1359, 1682, 2930, 3446. ¹H NMR (CDCl₃, 300 MHz): δ 0.63 (s, 3H, Me-18), 1.01 (s, 3H, Me-19), 2.12 (s, 3H, Me-21), 0.93-2.34 (m, 19H), 2.53 (m, 1H, H-17), 3.52 (m, 1H, H-3), 5.35 (m, 1H, H-6). ¹³C NMR (CDCl₃, 75 MHz): δ 13.4, 19.5, 21.3, 23.0, 24.6, 31.7, 31.8, 31.9, 32.0, 36.7, 37.4, 39.0, 42.4, 44.2, 50.2, 57.1, 63.9, 71.9, 121.5, 140.9, 209.7. MS (APCI+) m/z 339.0 (M+Na)⁺, 655.0 (2M+Na)⁺.

### Example 12

### Synthesis of 3β-Fluoropregn-5-en-20-one (APR-16)

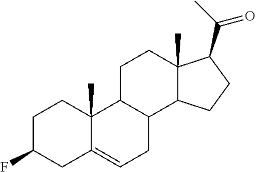 [SMILES: CC(=O)[C@H]1CCC2C3CC=C4C[C@@H](F)CC[C@]4(C)C3CC[C@@]21C]

At −78° C., the DAST (348 μL, 2.84 mmol) was added to a solution of (APR-15) (0.6 g, 1.89 mmol) in dry dichloromethane (12 mL), and the solution was stirred at room temperature for 20 min under argon. The reaction was quenched by pouring it into ice water and by washing the organic layer thoroughly with saturated sodium bicarbonate solution, followed by water. The solution was evaporated under reduced pressure. The crude product was chromatographed on silica gel (eluant: cyclohexane/EtOAc, 98/02) to afford one diastereoisomer 3β-fluoropregn-5-en-20-one (APR-16) (460 mg, 76% yield) as a white solid. R-0.56 (eluant: cyclohexane/EtOAc, 80/20). Mp 166-167° C. (lit. 155-160° C.). IR (v cm⁻¹): 836, 952, 1008, 1355, 1449, 1695, 2949. ¹H NMR (CDCl₃, 300 MHz): δ 0.63 (s, 3H, Me-18), 1.03 (s, 3H, Me-19), 2.12 (s, 3H, Me-21), 0.73-2.23 (m, 17H), 2.44 (m, 2H, H-4), 2.52 (m, 1H, H-17), 4.38 (dm, 1H, J_{HF}=50.5 Hz, Hα-3), 5.39 (m, 1H, H-6). ¹³C NMR (CDCl₃, 75 MHz): δ 13.4, 19.5, 21.3, 23.0, 24.6, 28.9 (d, ²J_{CF}=17.7 Hz), 29.9, 31.7, 31.9, 32.0, 36.5 (d, ³J_{CF}=10.8 Hz), 36.7, 39.0, 39.5 (d, ²J_{CF}=19.5 Hz), 44.1, 50.0, 57.0, 92.8 (d, ¹J_{CF}=174.1 Hz), 122.9, 139.5 (d, ³J_{CF}=12.4 Hz), 209.6. ¹⁹F NMR: 6-168.00 (d, 1F, J=50.5 Hz). MS (APCI+) m/z 341.0 (M+Na)⁺.

### Example 13

### Synthesis of 20-Ethynyl-3β-fluoropregn-5-en-20-ol (APR-21)

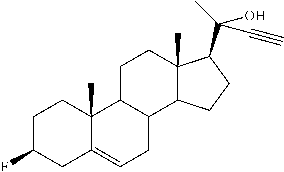 [SMILES: C#CC(C)(O)[C@H]1CCC2C3CC=C4C[C@@H](F)CC[C@]4(C)C3CC[C@@]21C]

To a suspension of LiC≡CH·EDA (179 mg, 1.94 mmol) in anhydrous THF (5 mL) at −78° C. was added dropwise a solution of (APR-16) (62 mg, 194 μmol) in THF (5 mL). The mixture was stirred 48 h at room temperature, quenched with saturated aq NH₄Cl, and extracted with CH₂Cl₂. The combined organic phases were dried over Na₂SO4. Filtration and solvent evaporation afforded a residue, which was chromatographed on silica gel (eluant: cyclohexane/EtOAc, 98/02) to afford (APR-21) (42 mg, 63% yield) as a white solid. R_{f}=0.44 (eluant: cyclohexane/EtOAc, 80/20). Mp 213-214° C. IR (v cm⁻¹): 799, 1010, 1454, 2087, 2925, 3274. ¹H NMR (CDCl₃, 300 MHz): δ 0.98 (s, 3H, Me-18), 1.03 (s, 3H, Me-19), 1.51 (s, 3H, CH₃), 0.83-2.00 (m, 17H), 2.16 (m, 1H), 2.44 (m, 2H), 2.51 (s, 1H, H_{C≡C}), 4.38 (dm, 1H, J_{HF}=50.7 Hz, Hα-3), 5.39 (m, 1H, H-6). ¹³C NMR (CDCl₃, 75 MHz): δ 13.5, 19.5, 21.0, 24.4, 25.3, 28.9 (d, ²J_{CF}=17.6 Hz), 31.5, 32.0, 32.9, 36.5 (d, ³J_{CF}=10.8 Hz), 36.7, 39.5 (d, ²J_{CF}=19.2 Hz), 40.3, 43.4, 50.1, 56.4, 60.1, 71.4, 74.0, 87.8, 92.9 (d, ¹J_{CF}=174.0 Hz), 122.9, 139.6 (d, ³J_{CF}=12.5 Hz). MS (APCI+) m/z 327.0 [M−(H₂O)+H]⁺.

### Example 14

### Synthesis of Pregn-5-en-3β,20-diol (APR-18)

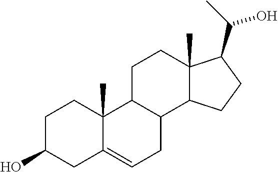 [SMILES: C[C@H](O)[C@H]1CCC2C3CC=C4C[C@@H](O)CC[C@]4(C)C3CC[C@@]21C]

The same procedure for the synthesis of (APR-09) was followed. From (4), the compound (APR-18) was obtained after chromatography on silica gel (eluant: cyclohexane/EtOAc, 60/40) in 96% yield as a mixture (3:7, α:β). R_{f}=0.39 (eluant: cyclohexane/EtOAc, 40/60). White solid. IR (v cm⁻¹): 1052, 1375, 2139, 2365, 2932, 3299, 3396. From the ¹H NMR and ¹³C NMR data this product was determined to be 3:7 mixture of epimers at C₂₀, the ¹H NMR (CDCl₃, 300 MHz) for the major β-isomer were δ 0.77 (s, 3H, Me-18), 1.01 (s, 3H, Me-19), 1.14 (d, 3H, J=5.9 Hz, Me-21), 0.91-2.27 (m, 20H), 3.52 (m, 1H, H-3), 3.73 (m, 1H, H-20), 5.35 (m, 1H, H-6). The ¹³C NMR (CDCl₃, 75 MHz) for the major β-isomer were δ 12.5, 19.6, 21.1, 23.8, 24.7, 25.8, 31.8, 31.9, 32.1, 37.4, 40.1, 42.5, 50.3, 56.4, 58.7, 70.7, 71.9, 121.7, 141.0. MS (APCI+) m/z 341.0 (M+Na)⁺.

### Example 15

### Synthesis of 5α-Pregnan-3β,20-diol (APR-29)

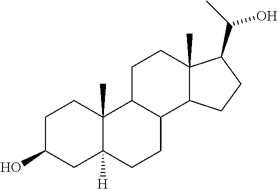 [SMILES: C[C@H](O)[C@H]1CCC2C3CC[C@H]4C[C@@H](O)CC[C@]4(C)C3CC[C@@]21C]

The same procedure for the synthesis of (5) was followed. From (APR-18), the compound (APR-29) was obtained in 96% yield as a mixture (3:7, α:β). R_{f}=0.39 (eluant: cyclohexane/EtOAc, 40/60). White solid. IR (v cm⁻¹): 1032, 1369, 2175, 2932, 3277, 3400. From the ¹H NMR and ¹³C NMR data this product was determined to be 3:7 mixture of epimers at C₂₀, the ¹H NMR (CDCl₃, 300 MHz) for the major β-isomer were δ 0.74 (s, 3H, Me-18), 0.81 (s, 3H, Me-19), 1.13 (d, 3H, J=6.1 Hz, Me-21), 0.60-2.04 (m, 23H), 3.59 (m, 1H, H-3α), 3.72 (m, 1H, H-20). The ¹³C NMR (CD₃OD, 75 MHz) for the major β-isomer were δ 12.8, 22.3, 23.8, 25.6, 26.9, 30.0, 32.2, 33.5, 36.7, 36.9, 38.3, 39.0, 41.1, 43.8, 46.3, 56.1, 57.5, 59.5, 70.9, 71.9. MS (APCI+) m/z 343.0 (M+Na)⁺.

### Example 16

### Synthesis of 3β,17α-Difluoro-17α-methyl-D-Homo-pregn-5-ene (APR-19)

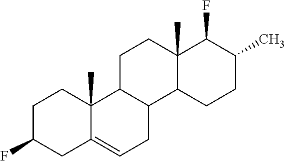 [SMILES: C[C@@H]1CCC2C3CC=C4C[C@@H](F)CC[C@]4(C)C3CC[C@]2(C)[C@H]1F]

At −78° C., the DAST (139 μL, 1.13 mmol) was added to a solution of (APR-18) (120 mg, 376 μmol) in dry dichloromethane (10 mL), and the solution was stirred at room temperature for 14 h under argon. The reaction was quenched by pouring it into ice water and by washing the organic layer thoroughly with saturated sodium bicarbonate solution, followed by water. The solution was evaporated under reduced pressure. The crude product was chromatographed on silica gel (eluant: petroleum ether/EtOAc, 99/01) to afford one diastereoisomere (APR-19) (83 mg, 69% yield) as a white solid. R_{f}=0.54 (eluant: petroleum ether/EtOAc, 95/05). Mp 131-132° C. IR (v cm⁻¹): 800, 950, 1003, 1382, 1442, 2918. ¹H NMR (CDCl₃, 300 MHz): δ 0.89 (s, 3H, Me-18), 0.98 (d, 3H, J=6.1 Hz, CH₃), 1.01 (s, 3H, Me-19), 0.68-2.17 (m, 18H), 2.45 (m, 2H, H-4), 3.60 (dd, 1H, ²J_{HF}=49.2, ³J_{HH}=10.2 Hz, H₁₇ₐ), 4.38 (dm, 1H, J_{HF}=50.4 Hz, Hα-3), 5.38 (m, 1H, H-6). ¹³C NMR (CDCl₃, 75 MHz): 11.9 (d, ³J_{CF}=3.1 Hz), 18.6 (d, ³J_{CF}=2.1 Hz), 19.4, 19.8, 23.5, 28.9 (d, ²J_{CF}=17.7 Hz), 31.2, 32.1, 32.3 (d, ²J_{CF}=18.0 Hz), 32.9 (d, ³J_{CF}=9.8 Hz), 36.2 (d, ³J_{CF}=10.8 Hz), 36.8, 36.9 (d, ²J_{CF}=18.0 Hz), 38.5 (d, ²J_{CF}=17.0 Hz), 39.3 (d, ²J_{CF}=19.3 Hz), 49.5, 49.9 (d, ³J_{CF}=5.0 Hz), 92.9 (d, ¹J_{CF}=174.0 Hz), 106.1 (d, ¹J_{CF}=181.1 Hz), 122.8, 139.3 (d, ³J_{CF}=12.5 Hz). MS (APCI+) m/z 303.0 [M−(HF)+H]⁺, 283.0 [M−(2HF)+H]⁺.

### Example 17

### Synthesis of 3β-Fluoro-5α-pregnan-20-one (APR-17)

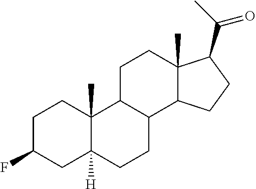 [SMILES: CC(=O)[C@H]1CCC2C3CC[C@H]4C[C@@H](F)CC[C@]4(C)C3CC[C@@]21C]

The same procedure for the synthesis of (5) was followed. From (APR-16), the compound (APR-17) was obtained after chromatography on silica gel (eluant: cyclohexane/EtOAc, 95/05) in 86% yield as a white solid. R_{f}=0.73 (eluant: cyclohexane/EtOAc, 80/20). Mp 154-155° C. IR (v cm⁻¹): 1018, 1150, 1356, 1705, 2162, 2931. ¹H NMR (CDCl₃, 300 MHz): δ 0.61 (s, 3H, Me-18), 0.83 (s, 3H, Me-19), 2.11 (s, 3H, Me-21), 0.63-2.20 (m, 22H), 2.51 (m, 1H, H-17), 4.47 (dm, 1H, J_{HF}=49.6 Hz, Hα-3). ¹³C NMR (CDCl₃, 75 MHz): δ 12.4, 13.6, 21.5, 23.0, 24.6, 28.6, 28.7 (d, ²J_{CF}=17.9 Hz), 31.6, 32.1, 35.2 (d, ²J_{CF}=16.8 Hz), 35.6, 36.5 (d, ³J_{CF}=11.1 Hz), 39.2, 44.4 (d, ³J_{CF}=9.9 Hz), 54.3, 56.8, 64.0, 92.9 (d, ¹J_{CF}=172.0 Hz), 209.7. MS (APCI+) m/z 343.0 (M+Na)⁺.

### Example 18

### Synthesis of 3β-Fluoro-5α-pregnan-20-ol (APR-25)

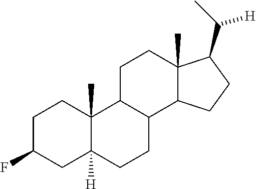

The same procedure for the synthesis of (APR-09) was followed. From (APR-17), the compound (APR-25) was obtained in 85% yield as a mixture (14:86, α:β). White solid. IR (v cm⁻¹): 876, 936, 1018, 1079, 1373, 1448, 2930, 3396. 3β-Fluoro-5α-pregnan-20β-ol: Rf=0.42 (eluant: petroleum ether/EtOAc, 8/2). White solid. Mp 118-119° C. ¹H NMR (CDCl₃, 300 MHz): δ 0.74 (s, 3H, Me-18), 0.83 (s, 3H, Me-19), 1.13 (d, 3H, J=4.8 Hz, Me-21), 0.60-2.05 (m, 23H), 3.73 (m, 1H, H-20), 4.47 (dm, 1H, J_{HF}=49.7 Hz, Hα-3). ¹³C NMR (CDCl₃, 75 MHz): δ 12.4, 12.7, 21.3, 23.8, 24.6, 25.8, 28.7 (d, ²J_{CF}=17.8 Hz), 28.8, 32.2, 35.3 (d, ²J_{CF}=16.5 Hz), 35.5, 35.6, 36.5 (d, ³J_{CF}=11.2 Hz), 40.2, 42.7, 44.4 (d, ³J_{CF}=9.7 Hz), 54.4, 56.1, 58.8, 70.7, 93.0 (d, ¹J_{CF}=171.9 Hz). MS (ESI+) m/z 345.0 (M+Na)⁺. 3β-Fluoro-5α-pregnan-20α-ol: Rf=0.28 (eluant: petroleum ether/EtOAc, 8/2). White solid. Mp 143-144° C. ¹H NMR (CDCl₃, 300 MHz): δ 0.65 (s, 3H, Me-18), 0.82 (s, 3H, Me-19), 1.21 (d, 3H, J=5.9 Hz, Me-21), 0.85-1.95 (m, 23H), 3.70 (m, 1H, H-20), 4.47 (dm, 1H, J_{HF}=49.3 Hz, Hα-3). ¹³C NMR (CDCl₃, 75 MHz): δ 12.4, 12.8, 21.1, 23.7, 24.2, 25.9, 28.7 (d, ²J_{CF}=17.9 Hz), 28.7, 32.1, 35.2 (d, ²J_{CF}=16.8 Hz), 35.2, 35.6, 36.4 (d, ³J_{CF}=11.4 Hz), 39.1, 42.0, 44.3 (d, ³J_{CF}=9.9 Hz), 54.3, 56.4, 58.7, 70.5, 92.9 (d, ¹J_{CF}=171.8 Hz). MS (ESI+) m/z 345.0 (M+Na)⁺.

### Example 19

### Synthesis of 3β,17aβ-Difluoro-17α-methyl-D-Homo-5α-androstane (APR-37)

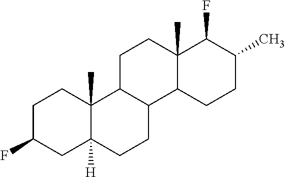 [SMILES: C[C@@H]1CCC2C3CC[C@H]4C[C@@H](F)CC[C@]4(C)C3CC[C@]2(C)[C@H]1F]

The same procedure for the synthesis of (APR-19) was followed. From (APR-25), the compound (APR-37) was obtained after chromatography on silica gel (eluant: cyclohexane/EtOAc, 99/01) in 60% yield as a white solid. R_{f}=0.57 (eluant: cyclohexane/EtOAc, 80/20). Mp 116-117° C. IR (v cm⁻¹): 837, 1022, 1387, 1448, 2876, 2935. ¹H NMR (CDCl₃, 300 MHz): δ 0.81 (s, 3H, Me-18), 0.85 (s, 3H, Me-19), 0.96 (d, 3H, J=5.9 Hz, CH₃), 0.62-1.98 (m, 23H), 3.57 (dd, 1H, ²J_{HF}=49.7, ³J_{HH}=10.5 Hz, H₁₇ₐ), 4.46 (dm, 1H, J_{HF}=49.8 Hz, Hα-3). ¹³C NMR (CDCl₃, 75 MHz): 12.0 (d, ³J_{CF}=3.3 Hz), 12.4, 18.7, 20.2, 23.3, 28.6 (d, ²J_{CF}=17.9 Hz), 28.7, 31.5, 32.1 (d, ²J_{CF}=17.9 Hz), 32.9 (d, ³J_{CF}=9.5 Hz), 34.7, 35.2 (d, ²J_{CF}=16.9 Hz), 35.8, 36.3 (d, ³J_{CF}=11.3 Hz), 37.2, 38.8 (d, ²J_{CF}=16.5 Hz), 43.9 (d, ³J_{CF}=10.0 Hz), 49.6 (d, ³J_{CF}=4.8 Hz), 53.8, 92.9 (d, ¹J_{CF}=171.7 Hz), 106.3 (d, ¹J_{CF}=181.0 Hz). MS (APCI+) m/z m/z 305.0 [M−(HF)+H]⁺, 285.0 [M−(2HF)+H]⁺.

### Example 20

### Synthesis of 3β-Fluoropregn-5-en-20-ol (APR-24)

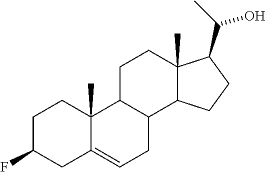 [SMILES: C[C@H](O)[C@H]1CCC2C3CC=C4C[C@@H](F)CC[C@]4(C)C3CC[C@@]21C]

The same procedure for the synthesis of (APR-09) was followed. From (APR-16), the compound (APR-24) was obtained in 94% yield as a mixture (13:87, α:β). White solid. IR (v cm⁻¹): 894, 954, 1011, 1375, 1447, 2942, 3380. 3β-Fluoropregn-5-en-20β-ol: Rf=0.46 (eluant: petroleum ether/EtOAc, 8/2). White solid. Mp 177-1780. ¹H NMR (CDCl₃, 300 MHz): δ 0.77 (s, 3H, Me-18), 1.03 (s, 3H, Me-19), 1.14 (d, 3H, J=5.7 Hz, Me-21), 0.90-2.17 (m, 18H), 2.43 (m, 2H), 3.74 (m, 1H, H-20), 4.38 (dm, 1H, J_{HF}=50.5 Hz, Hα-3), 5.38 (m, 1H, H-6). ¹³C NMR (CDCl₃, 75 MHz): δ 12.5, 19.5, 21.1, 23.8, 24.7, 25.8, 28.9 (d, ²J_{CF}=17.6 Hz), 31.8, 32.1, 36.5 (d, ³J_{CF}=10.8 Hz), 36.7, 39.5 (d, ²J_{CF}=19.2 Hz), 40.0, 42.4, 50.1, 56.3, 58.6, 70.7, 93.0 (d, ¹J_{CF}=174.0 Hz), 123.0, 139.6 (d, ³J_{CF}=12.5 Hz). MS (ESI+) m/z 343.0 (M+Na)⁺. 3β-Fluoropregn-5-en-20α-ol: Rf=0.31 (eluant: petroleum ether/EtOAc, 8/2). White solid. Mp 166-1670. ¹H NMR (CDCl₃, 300 MHz): δ 0.68 (s, 3H, Me-18), 1.05 (s, 3H, Me-19), 0.82-2.02 (m, 21H), 2.44 (m, 2H), 3.78 (m, 1H, H-20), 4.38 (dm, 1H, J_{HF}=50.5 Hz, Hα-3), 5.39 (m, 1H, H-6). ¹³C NMR (CDCl₃, 75 MHz): δ 12.6, 19.4, 20.9, 23.8, 24.3, 25.8, 28.9 (d, ²J_{CF}=17.4 Hz), 31.6, 32.0, 36.5 (d, ³J_{CF}=10.8 Hz), 36.7, 38.9, 39.5 (d, ²J_{CF}=19.2 Hz), 41.8, 50.0, 56.6, 58.6, 70.5, 92.9 (d, ¹J_{CF}=173.9 Hz), 123.0, 139.4 (d, ³J_{CF}=12.7 Hz). MS (ESI+) m/z 343.0 (M+Na)⁺.

### Example 21

### Synthesis of 20-Ethynyl-3β-fluoro-5α-pregnan-20-ol (APR-26)

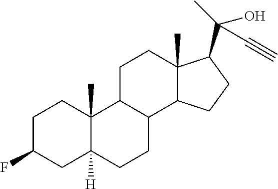 [SMILES: C#CC(C)(O)[C@H]1CCC2C3CC[C@H]4C[C@@H](F)CC[C@]4(C)C3CC[C@@]21C]

The same procedure for the synthesis of (APR-42) was followed with ethynylmagnesium bromide. From (APR-17), the compound (APR-26) was obtained after chromatography on silica gel (eluant: cyclohexane/EtOAc, 99/01) in 78% yield as a white solid. R_{f}=0.45 (eluant: cyclohexane/EtOAc, 80/20). Mp 230-231° C. IR (v cm⁻¹): 810, 1012, 1372, 1450, 1708, 2928, 3271. ¹H NMR (CDCl₃, 300 MHz): δ 0.62 (m, 1H), 0.83 (s, 3H, Me-18), 0.94 (s, 3H, Me-19), 1.49 (s, 3H, CH₃), 0.78-1.94 (m, 21H), 2.10 (m, 1H), 2.51 (s, 1H, H_{C≡C}), 4.56 (dm, 1H, J_{HF}=49.9 Hz, Hα-3). ¹³C NMR (CDCl₃, 75 MHz): δ 12.4, 13.7, 21.2, 24.3, 25.3, 28.7 (d, ²J_{CF}=17.8 Hz), 28.7, 32.1, 32.9, 35.0, 35.3 (d, ²J_{CF}=16.7 Hz), 35.6, 36.4 (d, ³J_{CF}=11.3 Hz), 40.6, 43.7, 44.4 (d, ³J_{CF}=9.8 Hz), 54.3, 56.1, 60.2, 71.4, 73.9, 87.8, 93.0 (d, ¹J_{CF}=171.8 Hz). MS (APCI+) m/z 329.0 [M−(H₂O)+H]⁺.

D—Synthesis of antagonists progesterone receptor (APRn) bearing a C3 fluorine atom (compounds of formula (I) wherein R₁═F and R′═H) from (+)-dehydro isoandrosterone:

The synthesis of 17-ethynyl APRn analogues with a C3 fluorine atom has been carried out using dehydro isoandrosterone as starting material (Scheme 4).

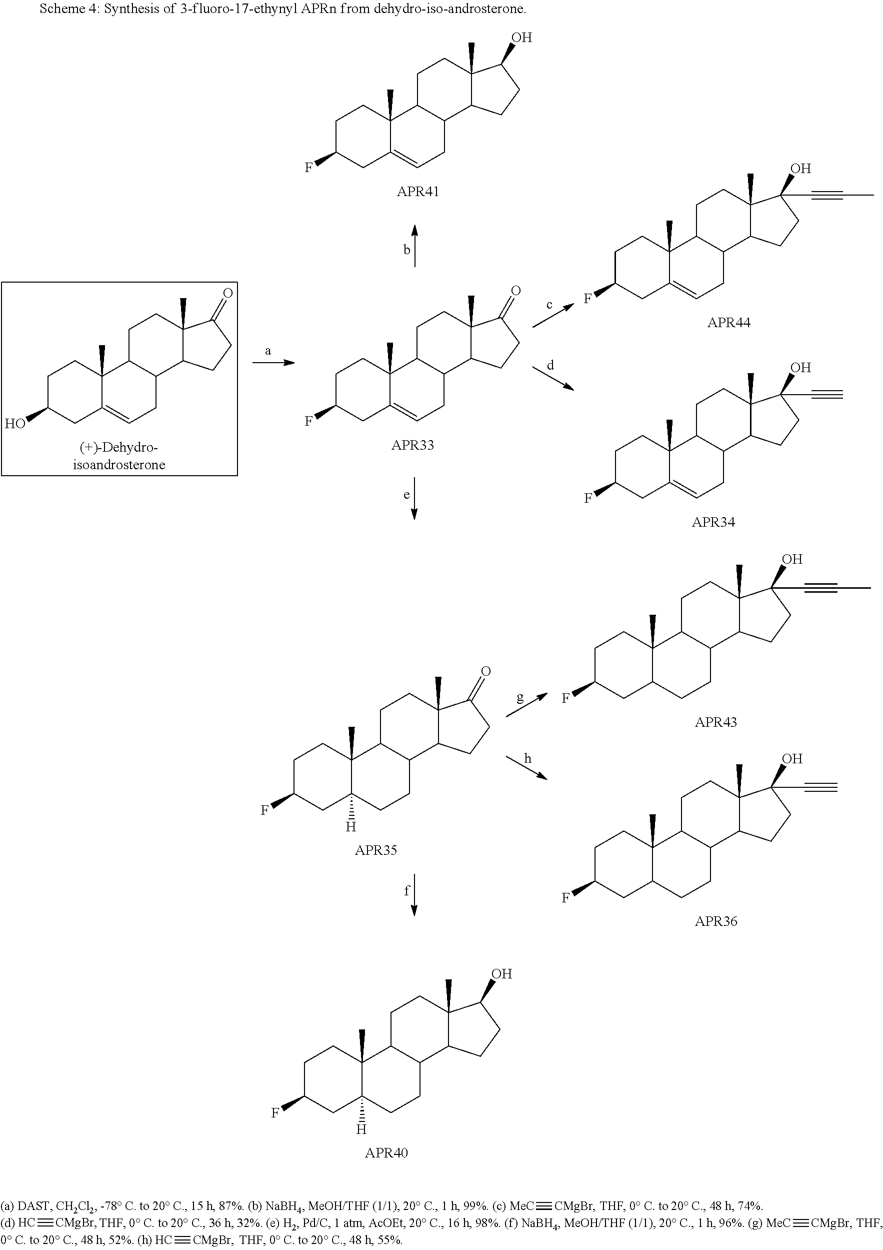

The preparation of the D⁵ APRn having a C3 fluoro atom began with the fluorination of alcohol function of (+)-dehydro isoandrosterone to give APR-33 (Scheme 4). The 17-keto function was then either subjected to selective reduction to give APR-41 or to react with metal acetylide to furnish acetylenic alcohols APR-34 and APR-44. For the synthesis of APRn analogues having no D⁵ double bond, APR-33 was initially reduced using H₂ in the presence of Pd/C to provide the 5a reduction product APR-35. Treatment of this latter with NaBH₄ in MeOH/THF led to selective reduction of the carbonyl function producing APR-40. Reaction of APR-35 with metal acetylide successfully forms acetylenic alcohols APR-36 and APR-43.

### Example 22

### Synthesis of 3β-Fluoroandrost-5-en-17-one (APR-33)

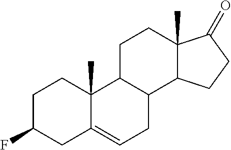 [SMILES: C[C@]12CC[C@H](F)CC1=CCC1C2CC[C@]2(C)C(=O)CCC12]

The same procedure for the synthesis of (APR-16) was followed. From (+)-dehydroisoandrosterone, the compound (APR-33) was obtained after chromatography on silica gel (eluant: cyclohexane/EtOAc, 95/05) in 71% yield as a white solid. R_{f}=0.36 (eluant: cyclohexane/EtOAc, 80/20). Mp 155-156° C. IR (v cm⁻¹): 846, 1006, 1023, 1380, 1456, 1737, 2941. ¹H NMR (CDCl₃, 300 MHz): δ 0.89 (s, 3H, Me-18), 1.05 (s, 3H, Me-19), 0.95-2.51 (m, 19H), 4.38 (dm, 1H, J_{HF}=50.4 Hz, Hα-3), 5.42 (m, 1H, H-6). ¹³C NMR (CDCl₃, 75 MHz): δ 13.7, 19.5, 20.6, 22.0, 28.8 (d, ²J_{CF}=17.7 Hz), 30.9, 31.57, 31.63, 36.0, 36.4 (d, ³J_{CF}=10.9 Hz), 36.8, 39.5 (d, ²J_{CF}=19.5 Hz), 47.7, 50.3, 51.9, 92.7 (d, ¹J_{CF}=174.3 Hz), 122.4, 139.8 (d, ³J_{CF}=12.7 Hz), 221.0. MS (APCI+) m/z 291.0 (M+H)⁺.

### Example 23

### Synthesis of 3β-Fluoro-androst-5-en-17β-ol (APR-41)

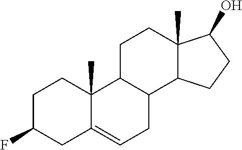 [SMILES: C[C@]12CC[C@H](F)CC1=CCC1C2CC[C@@]2(C)C1CC[C@@H]2O]

The same procedure for the synthesis of (APR-09) was followed. From (APR-33), the compound (APR-41) was obtained in 99% yield as a white solid. R_{f}=0.21 (eluant: cyclohexane/EtOAc, 50/50). Mp 165-166° C. IR (v cm⁻¹): 843, 952, 1025, 1442, 2198, 2939, 3348. ¹H NMR (CDCl₃, 300 MHz): δ 0.76 (s, 3H, Me-18), 1.04 (s, 3H, Me-19), 0.91-2.13 (m, 17H), 2.44 (m, 2H), 3.65 (m, 1H, H-17), 4.38 (dm, 1H, J_{HF}=50.5 Hz, Hα-3), 5.39 (m, 1H, H-6). ¹³C NMR (CDCl₃, 75 MHz): δ 11.1, 19.5, 20.9, 23.6, 28.9 (d, ²J_{CF}=17.6 Hz), 30.7, 31.7, 32.1, 36.5 (d, ³J_{CF}=10.8 Hz), 36.7, 39.6 (d, ²J_{CF}=19.2 Hz), 42.9, 50.3, 51.5, 82.0, 92.9 (d, ¹J_{CF}=174.0 Hz), 122.8, 139.6 (d, ³J_{CF}=12.4 Hz). MS (APCI+) m/z 275.0 [M−(H₂O)+H]⁺.

### Example 24

### Synthesis of 17α-Ethynyl-3β-fluoro-androst-5-en-17β-ol (APR-34)

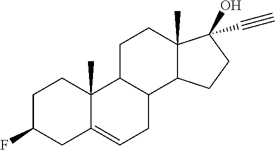 [SMILES: C#C[C@]1(O)CCC2C3CC=C4C[C@@H](F)CC[C@]4(C)C3CC[C@@]21C]

The same procedure for the synthesis of (APR-42) was followed with ethynylmagnesium bromide. From (APR-33), the compound (APR-34) was obtained after chromatography on silica gel (eluant: cyclohexane/EtOAc, 95/05) in 32% yield as a white solid. R_{f}=0.38 (eluant: cyclohexane/EtOAc, 80/20). Mp 219-220° C. IR (v cm⁻¹): 715, 803, 1011, 1366, 2936, 3285. ¹H NMR (CDCl₃, 300 MHz): δ 0.86 (s, 3H, Me-18), 1.04 (s, 3H, Me-19), 0.92-2.05 (m, 16H), 2.30 (m, 1H, H-16), 2.44 (m, 2H, H-4), 2.57 (s, 1H, H_{C≡C}), 4.38 (dm, 1H, J_{HF}=50.4 Hz, Hα-3), 5.39 (m, 1H, H-6). ¹³C NMR (CDCl₃, 75 MHz): δ 12.8, 19.5, 20.9, 23.3, 28.9 (d, ²J_{CF}=17.6 Hz), 31.6, 32.6, 32.7, 36.5 (d, ³J_{CF}=10.7 Hz), 36.7, 39.1, 39.5 (d, ²J_{CF}=19.2 Hz), 46.8, 49.8, 50.8, 74.1, 80.0, 92.9 (d, ¹J_{CF}=174.1 Hz), 122.8, 139.5 (d, ³J_{CF}=12.5 Hz). MS (ESI+) m/z 339.0 (M+Na)⁺.

### Example 25

### Synthesis of 3β-Fluoro-5α-androstan-17-one (APR-35)

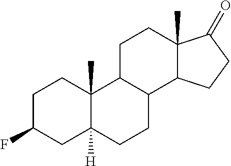 [SMILES: C[C@]12CCC3C(CC[C@H]4C[C@@H](F)CC[C@]34C)C1CCC2=O]

The same procedure for the synthesis of (5) was followed. From (APR-33), the compound (APR-35) was obtained in 86% yield as a white solid. R_{f}=0.55 (eluant: cyclohexane/EtOAc, 80/20). Mp 126-127° C. IR (v cm⁻¹): 986, 1059, 1373, 1453, 1739, 2854, 2934. ¹H NMR (CDCl₃, 300 MHz): δ 0.85 (s, 6H, Me-18, Me-19), 0.64-2.17 (m, 21H), 2.43 (m, 1H, H-16), 4.47 (dm, 1H, J_{HF}=49.5 Hz, Hα-3). ¹³C NMR (CDCl₃, 75 MHz): δ 12.4, 14.0, 20.7, 21.9, 28.5, 28.7 (d, ²J_{CF}=18.2 Hz), 31.0, 31.7, 35.1, 35.2 (d, ²J_{CF}=17.1 Hz), 35.8, 36.0, 36.4 (d, ³J_{CF}=11.3 Hz), 44.3 (d, ³J_{CF}=9.8 Hz), 47.9, 51.5, 54.5, 92.7 (d, ¹J_{CF}=172.0 Hz), 221.3. MS (ESI+) m/z 315.0 (M+Na)⁺.

### Example 26

### Synthesis of 3β-Fluoro-5α-androstan-17β-ol (APR-40)

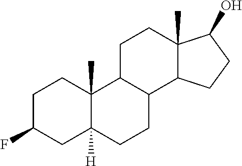 [SMILES: C[C@]12CCC3C(CC[C@H]4C[C@@H](F)CC[C@]34C)C1CC[C@@H]2O]

The same procedure for the synthesis of (APR-09) was followed. From (APR-35), the compound (APR-40) was obtained in 969% yield as a white solid. R_{f}=0.27 (eluant: cyclohexane/EtOAc, 80/20). Mp 150-151° C. IR (v cm⁻¹): 1015, 1052, 1356, 1448, 2010, 2159, 2924, 3261. ¹H NMR (CDCl₃, 300 MHz): δ 0.73 (s, 3H, Me-18), 0.84 (s, 3H, Me-19), 0.58-2.11 (m, 22H), 3.62 (m, 1H, H-17), 4.46 (dm, 1H, J_{HF}=49.7 Hz, Hα-3). ¹³C NMR (CDCl₃, 75 MHz): δ 11.3, 12.4, 21.0, 23.5, 28.7, 28.7 (d, ²J_{CF}=17.5 Hz), 30.7, 31.7, 35.3 (d, ²J_{CF}=16.8 Hz), 35.7, 36.5 (d, ³J_{CF}=11.4 Hz), 36.9, 43.1, 44.4 (d, ³J_{CF}=9.8 Hz), 51.1, 54.5, 82.1, 92.9 (d, ¹J_{CF}=172.0 Hz). MS (APCI+) m/z 277.0 [M−(H₂O)+H]⁺.

### Example 27

### Synthesis of 17α-Ethynyl-3β-fluoro-5α-androstan-17β-ol (APR-36)

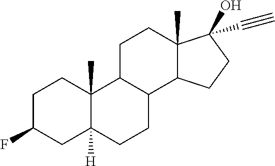 [SMILES: C#C[C@]1(O)CCC2C3CC[C@H]4C[C@@H](F)CC[C@]4(C)C3CC[C@@]21C]

The same procedure for the synthesis of (APR-42) was followed with ethynylmagnesium bromide. From (APR-35), the compound (APR-36) was obtained after chromatography on silica gel (eluant: cyclohexane/EtOAc, 95/05) in 55% yield as a white solid. R_{f}=0.39 (eluant: cyclohexane/EtOAc, 80/20). Mp 225-226° C. IR (v cm⁻¹): 795, 1007, 1079, 1373, 1453, 2932, 3287. ¹H NMR (CDCl₃, 300 MHz): δ 0.83 (s, 3H, Me-18), 0.84 (s, 3H, Me-19), 0.63-2.01 (m, 21H), 2.27 (m, 1H, H-16), 2.57 (s, 1H, H_{C≡C}), 4.46 (dm, 1H, J_{HF}=49.6 Hz, Hα-3). ¹³C NMR (CDCl₃, 75 MHz): δ 12.4, 12.9, 21.1, 23.2, 28.6, 28.7 (d, ²J_{CF}=15.8 Hz), 31.7, 32.8, 35.2 (d, ²J_{CF}=16.8 Hz), 35.7, 36.2, 36.5 (d, ³J_{CF}=11.2 Hz), 39.1, 44.4 (d, ³J_{CF}=9.9 Hz), 47.0, 50.5, 54.0, 74.0, 80.0, 87.7, 92.9 (d, ¹J_{CF}=171.9 Hz). MS (ESI+) m/z 341.0 (M+Na)⁺.

### Example 28

### Synthesis of 3β-Fluoro-17α-(1-propynyl)-5α-androstan-17β-ol (APR-43)

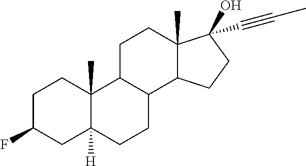 [SMILES: CC#C[C@]1(O)CCC2C3CC[C@H]4C[C@@H](F)CC[C@]4(C)C3CC[C@@]21C]

The same procedure for the synthesis of (APR-42) was followed. From (APR-35), the compound (APR-43) was obtained after chromatography on silica gel (eluant: cyclohexane/EtOAc, 95/05) in 52% yield as a white solid. R_{f}=0.39 (eluant: cyclohexane/EtOAc, 80/20). Mp 139-140°. IR (v cm⁻¹): 853, 937, 1009, 1073, 1132, 1374, 1439, 2854, 2920, 3532. ¹H NMR (CDCl₃, 300 MHz): δ 0.63-0.71 (m, 1H), 0.81 (s, 3H, Me-18), 0.84 (s, 3H, Me-19), 0.86-1.82 (m, 21H), 1.87 (s, 1H, Me), 1.89-1.98 (m, 2H), 2.15-2.24 (m, 1H), 4.46 (dm, 1H, J_{HF}=49.6 Hz, Hα-3). ¹³C NMR (CDCl₃, 75 MHz): δ 3.9, 12.4, 13.1, 21.1, 23.3, 28.7, 28.8 (d, ²J_{CF}=17.8 Hz), 31.7, 33.0, 35.3 (d, ²J_{CF}=16.9 Hz), 35.7, 36.3, 36.5 (d, ³J_{CF}=11.2 Hz), 39.2, 44.4 (d, ³J_{CF}=9.8 Hz), 47.0, 50.5, 54.0, 80.2, 81.8, 83.0, 92.9 (d, ¹J_{CF}=171.9 Hz). MS (APCI+) m/z 315.0 [M−(H₂O)+H]⁺.

### Example 29

### Synthesis of 31-Fluoro-17α-(1-propynyl)androst-5-en-17β-ol (APR-44)

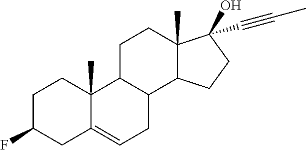 [SMILES: CC#C[C@]1(O)CCC2C3CC=C4C[C@@H](F)CC[C@]4(C)C3CC[C@@]21C]

The same procedure for the synthesis of (APR-42) was followed. From (APR-33), the compound (APR-44) was obtained after chromatography on silica gel (eluant: cyclohexane/EtOAc, 90/10) in 74% yield as a white solid. R_{f}=0.39 (eluant: cyclohexane/EtOAc, 80/20). Mp 136-137° C. IR (v cm⁻¹): 955, 1013, 1077, 1139, 1247, 1380, 1439, 2855, 2943, 3532. ¹H NMR (CDCl₃, 300 MHz): δ 0.84 (s, 3H, Me-18), 1.04 (s, 3H, Me-19), 0.87-1.77 (m, 12H), 1.86 (m, 3H, Me), 1.89-2.05 (m, 5H), 2.21 (m, 1H), 2.44 (m, 2H), 4.37 (dm, 1H, J_{HF}=50.5 Hz, Hα-3), 5.39 (m, 1H, H-6). ¹³C NMR (CDCl₃, 75 MHz): δ 3.9, 12.9, 19.5, 21.0, 23.4, 28.9 (d, ²J_{CF}=17.6 Hz), 31.6, 32.7, 32.9, 36.5 (d, ³J_{CF}=10.7 Hz), 36.7, 39.2, 39.6 (d, ²J_{CF}=19.3 Hz), 46.8, 49.8, 50.8, 80.2, 81.9, 82.9, 92.9 (d, ¹J_{CF}=174.1 Hz), 122.9, 139.6 (d, ³J_{CF}=12.6 Hz). MS (APCI+) m/z 313.0 [M−(H₂O)+H]⁺.

E—Synthesis of antagonists progesterone receptor (APRn) bearing a C3 methoxy or hydroxy substituent (compounds having formula (I) wherein R₁═OMe or OH and R′₁═H) from pregnenolone acetate:

Readily available pregnenolone acetate was also used as starting material for the synthesis of APRn analogues having at the C3 position a methoxy or a hydroxy substituent (Scheme 5).

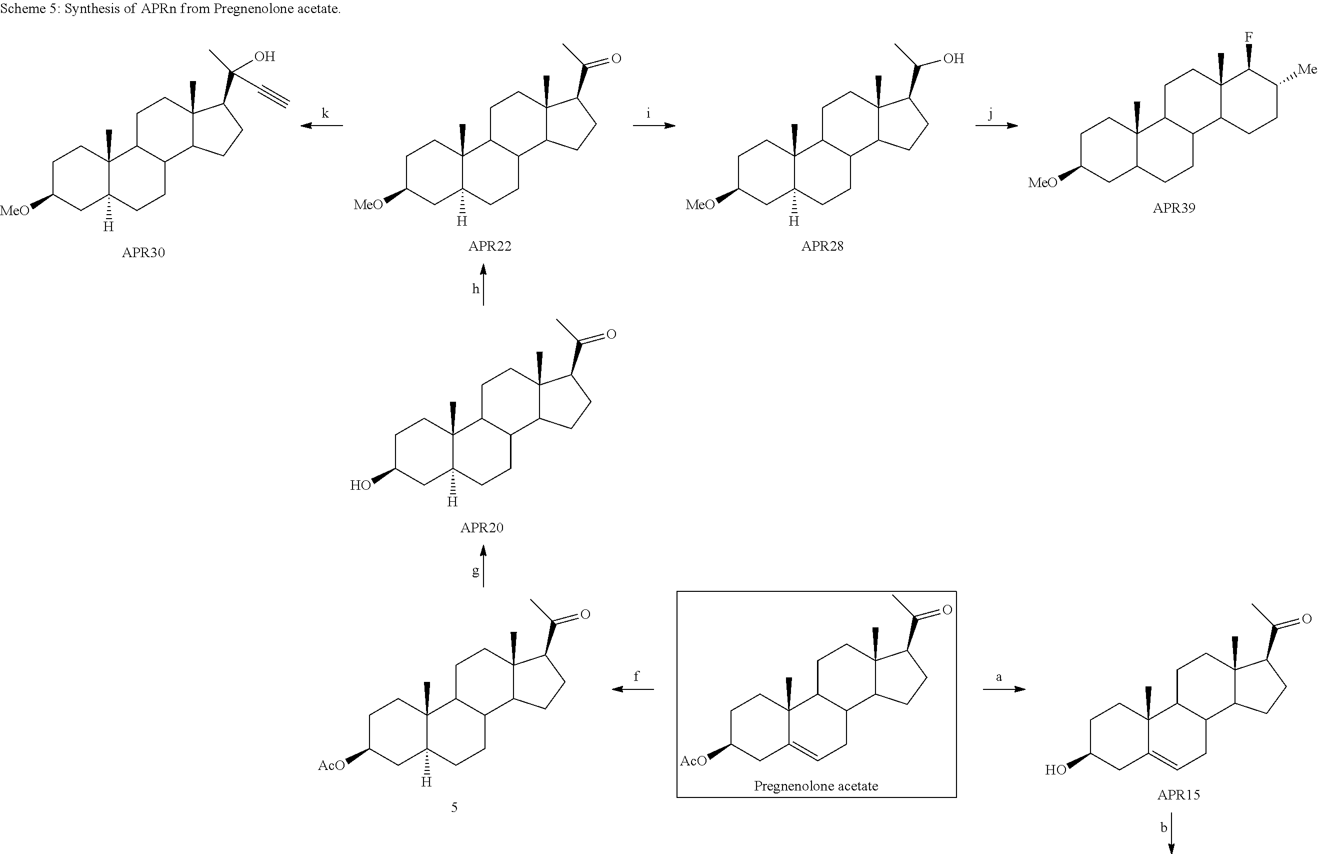

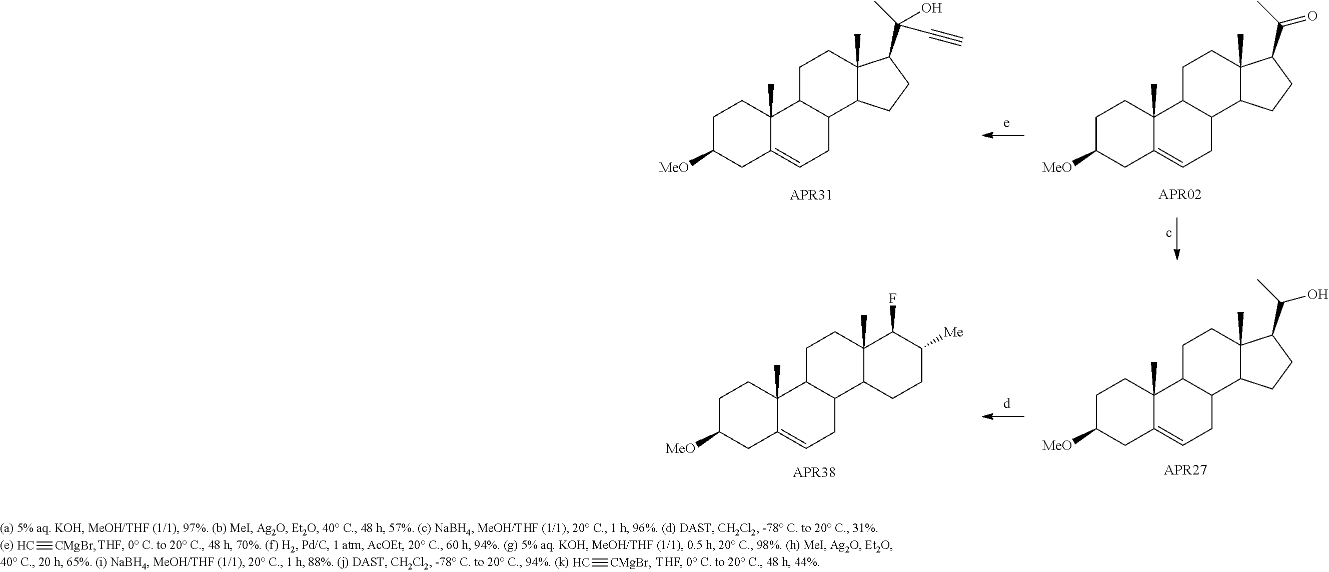

For the synthesis of D⁵ APRn analogues having a C3-methoxy substituent, the previously obtained APR-15 was reacted with methyl iodide using Ag₂O as the base to give APR-02, which was then reduced with NaBH₄ to afford APR-27. Further reaction in the presence of DAST furnished as expected homosteroid APR-38. Condensation of APR-02 with acetylenemagnesium bromide was also successful to give acetylene alcohol APR-31.

APRn analogues having a C3-methoxy substituent but with no D⁵ double bond have also been prepared (Scheme 3). To this end, the catalytic hydrogenation of D⁵ in pregnenolone acetate using Pd/C gave the 5a steroid 5 with trans stereochemistry at the A/B ring junction, which was deacetylated under alkaline conditions to provide APR-20. After alkylation of the C3 hydroxy group, the formed APR-22 was reduced with NaBH₄ in THF/MeOH (APR-28) and then was subjected to a rearrangement reaction in the presence of DAST to afford homosteroid APR-39. The same APR-22 was also reacted with acetylenemagnesium bromide to give steroid APR-30.

### Synthesis of 3β-Acetoxy-5α-pregnan-20-one (5)

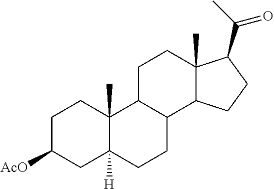 [SMILES: CC(=O)O[C@H]1CC[C@]2(C)C3CC[C@@]4(C)C(CC[C@@H]4C(C)=O)C3CC[C@H]2C1]

To a solution of pregnenolone acetate (2 g, 5.58 mmol) in ethyl acetate (30 mL) was added Pd/C catalyst (296 mg, 5%) and hydrogenation was carried at room temperature in atmospheric pression for 60 h. The reaction mixture was filtered and the filtrate was evaporated under reduced pressure. The crude product was chromatographed on silica gel (eluant: cyclohexane/EtOAc, 90/10) to afford one diastereoisomere 3β-acetoxy-5α-pregnan-20-one (5) (1.89 g, 94% yield) as a white solid. R_{f}=0.45 (eluant: cyclohexane/EtOAc, 80/20). Mp 141-142° C. (lit. 144-146° C.). IR (v cm⁻¹): 1031, 1258, 1367, 1706, 1728, 2937. ¹H NMR (CDCl₃, 300 MHz): δ 0.60 (s, 3H, Me-18), 0.82 (s, 3H, Me-19), 2.02 (s, 3H, CH₃), 2.10 (s, 3H, Me-21), 0.65-2.16 (m, 22H), 2.51 (m, 1H, H-17), 4.68 (m, 1H, H-3). ¹³C NMR (CDCl₃, 75 MHz): δ 12.3, 13.6, 21.3, 21.6, 23.0, 24.5, 27.6, 28.6, 31.6, 32.1, 34.1, 35.6, 35.7, 36.9, 39.2, 44.4, 44.8, 54.3, 56.8, 64.0, 73.8, 170.8, 209.7. MS (APCI+) m/z 383.0 (M+Na)⁺.

### Example 30

### Synthesis of 3β-Methoxypregn-5-en-20-one (APR-02)

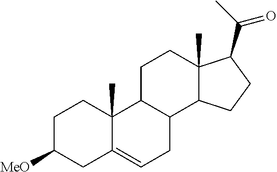 [SMILES: CO[C@H]1CC[C@@]2(C)C(=CCC3C2CC[C@@]2(C)C3CC[C@@H]2C(C)=O)C1]

General Procedure for Methylation:

(see Reymond, S.; Cossy, J. Tetrahedron 2007, 63, 5918)

To a solution of freshly prepared Ag₂O (2.19 g, 9.48 mmol) and activated MS 4 Å in Et₂O (100 mL) and THF (40 mL), was added the above alcohol (APR-15) (2 g, 6.32 mmol) followed by iodomethane (11.8 mL, 189 mmol). After 48 h at 40° C., the mixture was filtered on a pad of Celite and washed with Et₂O. The solvent was removed under reduced pressure. The crude product was chromatographed on silica gel (eluant: petroleum ether/EtOAc, 95/05) to afford (APR-02) (1.2 g, 57% yield) as a white solid. R_{f}=0.57 (eluant: cyclohexane/EtOAc, 80/20). Mp 123-124° C. (lit. 121-123° C.). IR (v cm⁻¹): 945, 1094, 1189, 1352, 1452, 1698, 2945. ¹H NMR (CDCl₃, 300 MHz): δ 0.63 (s, 3H, Me-18), 1.00 (s, 3H, Me-19), 2.12 (s, 3H, Me-21), 0.92-2.23 (m, 18H), 2.39 (m, 1H, H-4), 2.53 (m, 1H, H-17), 3.06 (m, 1H, Hα-3), 3.35 (s, 3H, OMe), 5.35 (m, 1H, H-6). ¹³C NMR (CDCl₃, 75 MHz): δ 13.4, 19.5, 21.2, 23.0, 24.6, 28.1, 31.7, 31.9, 32.0, 37.1, 37.3, 38.8, 39.0, 44.2, 50.2, 55.8, 57.1, 63.9, 80.4, 121.4, 141.0, 209.7. MS (APCI+) m/z 331.0 (M+H)⁺.

### Example 31

### Synthesis of 3β-Methoxypregn-5-en-20-ol (APR-27)

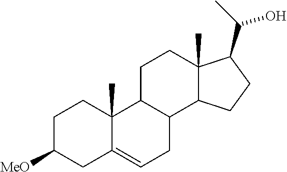 [SMILES: CO[C@H]1CC[C@@]2(C)C(=CCC3C2CC[C@@]2(C)C3CC[C@@H]2[C@H](C)O)C1]

The same procedure for the synthesis of (APR-09) was followed. From (APR-02), the compound (APR-27) was obtained in 96% yield as a mixture (3:7, α:β). IR (v cm⁻¹): 928, 1081, 1357, 2019, 2120, 2936, 3377. 3β-Methoxypregn-5-en-20β-ol: Rf=0.33 (eluant: petroleum ether/EtOAc, 8/2). White solid. Mp 152-153° C. ¹H NMR (CDCl₃, 300 MHz): δ 0.77 (s, 3H, Me-18), 1.01 (s, 3H, Me-19), 1.14 (d, 3H, J=5.9 Hz, Me-21), 0.71-2.20 (m, 19H), 2.36-2.42 (m, 1H), 3.06 (m, 1H, H-3), 3.36 (s, 3H, OMe), 3.74 (m, 1H, H-20), 5.35 (m, 1H, H-6). ¹³C NMR (CDCl₃, 75 MHz) δ 12.5, 19.5, 21.1, 23.8, 24.7, 25.8, 28.1, 31.9, 32.1, 37.1, 37.3, 38.9, 40.1, 42.4, 50.3, 55.7, 56.4, 58.6, 70.7, 80.5, 121.5, 141.1. MS (ESI+) m/z 355.0 (M+Na)⁺. 3β-Methoxypregn-5-en-20α-ol: Rf=0.28 (eluant: petroleum ether/EtOAc, 8/2). White solid. Mp 124-125° C. ¹H NMR (CDCl₃, 300 MHz): δ 0.68 (s, 3H, Me-18), 1.00 (s, 3H, Me-19), 1.23 (d, 3H, J=6.3 Hz, Me-21), 0.84-2.02 (m, 18H), 2.15 (m, 1H), 2.39 (m, 1H), 3.06 (m, 1H, H-3), 3.35 (s, 3H, OMe), 3.71 (m, 1H, H-20), 5.35 (m, 1H, H-6). ¹³C NMR (CDCl₃, 75 MHz) δ 12.6, 19.5, 20.9, 23.7, 24.3, 25.9, 28.1, 31.7, 32.0, 37.0, 37.3, 38.8, 38.9, 41.7, 50.3, 55.7, 56.7, 58.6, 70.4, 80.4, 121.6, 141.0. MS (ESI+) m/z 355.0 (M+Na)⁺.

### Example 32

### Synthesis of 20-Ethynyl-3β-methoxypregn-5-en-20-ol (APR-31)

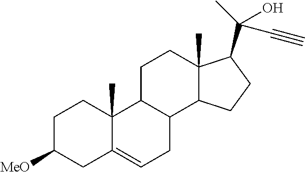 [SMILES: C#CC(C)(O)[C@H]1CCC2C3CC=C4C[C@@H](OC)CC[C@]4(C)C3CC[C@@]21C]

The same procedure for the synthesis of (APR-42) was followed with ethynylmagnesium bromide. From (APR-02), the compound (APR-31) was obtained after chromatography on silica gel (eluant: cyclohexane/EtOAc, 98/02) in 70% yield as a white solid. R_{f}=0.44 (eluant: cyclohexane/EtOAc, 80/20). Mp 175-176° C. IR (v cm⁻¹): 801, 936, 1015, 1081, 1366, 1451, 2933, 3455. ¹H NMR (CDCl₃, 300 MHz): δ 0.97 (s, 3H, Me-18), 1.01 (s, 3H, Me-19), 1.51 (s, 3H, Me-21), 0.79-2.03 (m, 17H), 2.16 (m, 2H), 2.38 (m, 1H), 2.51 (s, 1H, H_{C≡C}), 3.05 (m, 1H, Hα-3), 3.35 (s, 3H, OMe), 5.35 (m, 1H, H-6). ¹³C NMR (CDCl₃, 75 MHz): δ 13.5, 19.5, 21.0, 24.4, 25.3, 28.1, 31.5, 32.0, 32.9, 37.1, 37.3, 38.9, 40.4, 43.5, 50.3, 55.7, 56.5, 60.2, 71.4, 73.9, 80.5, 87.8, 121.5, 141.2. MS (APCI+) m/z 379.0 (M+Na)⁺.

### Example 33

### Synthesis of 17aβ-Fluoro-3β-methoxy-17α-methyl-D-Homo-pregn-5-ene (APR-38)

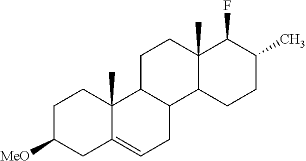 [SMILES: CO[C@H]1CC[C@@]2(C)C(=CCC3C2CC[C@@]2(C)C3CC[C@@H](C)[C@@H]2F)C1]

The same procedure for the synthesis of (APR-19) was followed. From (APR-27), the compound (APR-38) was obtained after chromatography on silica gel (eluant: cyclohexane/EtOAc, 99/01) in 96% yield as a white solid. R_{f}=0.55 (eluant: cyclohexane/EtOAc, 95/05). Mp 164-165° C. IR (v cm⁻¹): 984, 1095, 1191, 1367, 1456, 2933. ¹H NMR (CDCl₃, 300 MHz): δ 0.88 (s, 3H, Me-18), 0.97 (d, 3H, J=6.0 Hz, CH₃), 0.98 (s, 3H, Me-19), 0.68-2.19 (m, 19H), 2.39 (m, 1H, H-4), 3.05 (m, 1H, H-3), 3.35 (s, 3H, CH₃), 3.59 (dd, 1H, ²J_{HF}=49.2, ³J_{HH}=10.3 Hz, H₁₇ₐ), 5.33 (m, 1H, H-6). ¹³C NMR (CDCl₃, 75 MHz): 11.9 (d, ³J_{CF}=3.0 Hz), 18.6, 19.4, 19.8, 23.5, 28.1, 31.2, 32.1, 32.4, 32.9 (d, ³J_{CF}=9.5 Hz), 36.8, 37.0, 37.3, 38.3 (d, ³J_{CF}=9.6 Hz), 38.7, 49.7, 49.9 (d, ³J_{CF}=5.0 Hz), 55.7, 80.4, 106.1 (d, ¹J_{CF}=181.0 Hz), 121.3, 140.8. MS (APCI+) m/z 303.0 [M−(CH₃OH)+H]⁺.

### Example 34

### Synthesis of 3β-Hydroxy-5α-pregnan-20-one (APR-20)

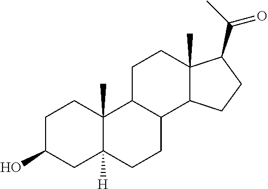 [SMILES: CC(=O)[C@H]1CCC2C3CC[C@H]4C[C@@H](O)CC[C@]4(C)C3CC[C@@]21C]

The same procedure for the synthesis of (2) was followed. From (5) the compound (APR-20) was obtained in 98% yield as a white solid. Mp 196-197° C. (lit. 194-196° C.). IR (v cm⁻¹): 1036, 1080, 1350, 1683, 1698, 2929. ¹H NMR (CDCl₃, 300 MHz): δ 0.60 (s, 3H, Me-18), 0.80 (s, 3H, Me-19), 2.10 (s, 3H, Me-21), 0.63-2.20 (m, 22H), 2.51 (m, 1H, H-17), 3.59 (m, 1H, Hα-3). ¹³C NMR (CDCl₃, 75 MHz): δ 12.5, 13.6, 21.4, 23.0, 24.6, 28.7, 31.6, 32.2, 35.7, 37.2, 38.3, 39.2, 44.4, 45.0, 54.4, 56.9, 64.0, 71.4, 209.8. MS (ESI+) m/z 341.0 (M+Na)⁺.

### Example 35

### Synthesis of 3β-Methoxy-5α-pregnan-20-one (APR-22)

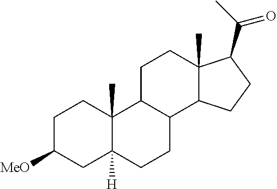 [SMILES: CO[C@H]1CC[C@]2(C)C3CC[C@@]4(C)C(CC[C@@H]4C(C)=O)C3CC[C@H]2C1]

The same procedure for the synthesis of (APR-02) was followed. From (APR-20), the compound (APR-22) was obtained after chromatography on silica gel (eluant: cyclohexane/EtOAc, 95/05) in 65% yield as a white solid. R_{f}=0.47 (eluant: cyclohexane/EtOAc, 80/20). Mp 125-126° C. IR (v cm⁻¹): 1092, 1352, 1701, 2845, 2923. ¹H NMR (CDCl₃, 300 MHz): δ 0.60 (s, 3H, Me-18), 0.79 (s, 3H, Me-19), 2.10 (s, 3H, Me-21), 0.63-2.20 (m, 22H), 2.51 (m, 1H, H-17), 3.12 (m, 1H, Hα-3), 3.34 (s, 3H, OMe). ¹³C NMR (CDCl₃, 75 MHz): δ 12.4, 13.6, 21.4, 23.0, 24.6, 28.0, 28.9, 31.6, 32.2, 34.5, 35.7, 36.0, 37.1, 39.3, 44.4, 45.0, 54.5, 55.7, 56.9, 64.0, 80.0, 209.7. MS (APCI+) m/z 333.0 (M+H)⁺.

### Example 36

### Synthesis of 3β-Methoxy-5α-pregnan-20-ol (APR-28)

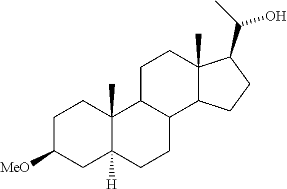 [SMILES: CO[C@H]1CC[C@]2(C)C3CC[C@@]4(C)C(CC[C@@H]4[C@H](C)O)C3CC[C@H]2C1]

The same procedure for the synthesis of (APR-09) was followed. From (APR-22), the compound (APR-28) was obtained in 88% yield as a mixture (3:7, α:β). White solid. IR (v cm⁻¹): 969, 1094, 1372, 1448, 2094, 2921, 3488. From the ¹H NMR and ¹³C NMR data this product was determined to be 3:7 mixture of epimers at C₂₀, the ¹H NMR (CDCl₃, 300 MHz) for the major β-isomer were δ 0.74 (s, 3H, Me-18), 0.80 (s, 3H, Me-19), 1.13 (d, 3H, J=6.2 Hz, Me-21), 0.60-2.04 (m, 23H), 3.12 (m, 1H, H-3α), 3.34 (s, 3H, OMe), 3.72 (m, 1H, H-20). The ¹³C NMR (CD₃OD, 75 MHz) for the major β-isomer were δ 12.4, 12.7, 21.3, 23.7, 24.6, 25.8, 28.0, 29.0, 32.3, 34.5, 35.5, 36.0, 37.1, 40.3, 42.7, 44.9, 54.5, 55.7, 56.1, 58.7, 70.7, 80.0. MS (APCI+) m/z 357.0 (M+Na)⁺.

### Example 37

### Synthesis of 17aβ-Fluoro-3β-methoxy-17α-methyl-D-Homo-5α-androstane (APR-39)

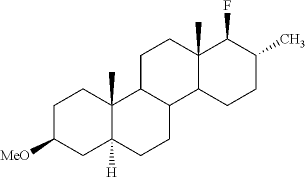 [SMILES: CO[C@H]1CC[C@]2(C)C3CC[C@@]4(C)C(CC[C@@H](C)[C@@H]4F)C3CC[C@H]2C1]

The same procedure for the synthesis of (APR-19) was followed. From (APR-28), the compound (APR-39) was obtained after chromatography on silica gel (pentane/EtOAc, 998/002) in 60% yield as a white solid. R_{f}=0.69 (pentane/EtOAc, 95/5). Mp 132-133° C. IR (v cm⁻¹): 838, 1021, 1105, 1385, 1446, 2848, 2921. ¹H NMR (CDCl₃, 300 MHz): δ 0.77 (s, 3H, Me-18), 0.85 (s, 3H, Me-19), 0.96 (d, 3H, J=6.1 Hz, CH₃), 0.61-1.97 (m, 23H), 3.12 (m, 1H, H-3), 3.33 (s, 3H, CH₃), 3.58 (dd, 1H, ²J_{HF}=49.3, ³J_{HH}=10.2 Hz, H₁₇ₐ). ¹³C NMR (CDCl₃, 75 MHz): 12.0 (d, ³J_{CF}=2.8 Hz), 12.4, 18.7 (d, ³J_{CF}=1.9 Hz), 20.1, 23.3, 27.9, 28.9, 31.5, 32.1 (d, ²J_{CF}=17.9 Hz), 32.9 (d, ³J_{CF}=9.5 Hz), 34.4, 34.7, 36.1, 36.9, 37.3, 38.8 (d, ²J_{CF}=16.0 Hz), 44.4, 49.6 (d, ³J_{CF}=4.8 Hz), 54.0, 55.7, 79.9, 106.4 (d, ¹J_{CF}=180.9 Hz). MS (APCI+) m/z 305.0 [M−(CH₃OH)+H]⁺, 285.0 [M−(CH₃OH)—(HF)+H]⁺.

### Example 38

### Synthesis of 20-Ethynyl-3β-methoxy-5α-pregnan-20-ol (APR-30)

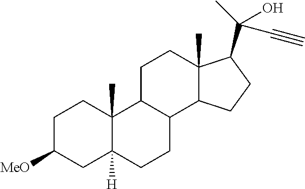 [SMILES: C#CC(C)(O)[C@H]1CCC2C3CC[C@H]4C[C@@H](OC)CC[C@]4(C)C3CC[C@@]21C]

The same procedure for the synthesis of (APR-42) was followed with ethynylmagnesium bromide. From (APR-22), the compound (APR-30) was obtained after chromatography on silica gel (eluant: cyclohexane/EtOAc, 98/02) in 44% yield as a white solid. R_{f}=0.47 (eluant: cyclohexane/EtOAc, 80/20). Mp 168-169. IR (v cm⁻¹): 923, 1086, 1369, 1447, 2027, 2926, 3393. ¹H NMR (CDCl₃, 300 MHz): δ 0.80 (s, 3H, Me-18), 0.94 (s, 3H, Me-19), 1.49 (s, 3H, Me-21), 0.58-1.89 (m, 22H), 2.09 (m, 1H), 2.50 (s, 1H, H_{C≡C}), 3.12 (m, 1H, Hα-3), 3.33 (s, 3H, OMe). ¹³C NMR (CDCl₃, 75 MHz): δ 12.4, 13.8, 21.2, 24.3, 25.3, 28.0, 29.0, 32.2, 32.9, 34.5, 35.1, 36.0, 37.1, 40.7, 43.7, 45.0, 54.6, 55.7, 56.2, 60.3, 71.4, 73.9, 80.0, 87.9. MS (APCI+) m/z 341.0 [M−(H₂O)+H]⁺.

F—Synthesis of antagonists progesterone receptor (APRn) bearing a C3 methoxy or hydroxy substituent (compounds having formula (I) wherein R₁═OMe or OH and R′₁═H) from progesterone:

The synthesis of fluoro homosteroid APR-07 was carried out from progesterone (Scheme 6). Both ketone groups were protected as ethylene ketal, yielding known steroid 6 (Sondheimer, F.; Velasco, M.; Rosenkranz, G. J. Am. Chem. Soc. 1955, 77, 192-194). It was reported that the cleavage of 1,3-dioxolanes in conjugated enone systems is faster than in saturated dioxolanes. All our attempts to achieve the selective deacetalization at the C3 position in the presence of cerium(III) chloride (Marcantoni, E.; Nobili, F. J. Org. Chem. 1997, 62, 4183-4184), magnesium sulfate (Brown, J. J.; Lenhard, R. H.; Bernstein, S. J. Am. Chem. Soc. 1964, 86, 2183-2187), or wet silica gel (Huet, F.; Lechevallier, A.; Pellet, M.; Conia, J. M. Synthesis 1978, 63-65) resulted in unsuccessful results. After a series of experiments, it has been found that the use silica gel in the presence of oxalic acid (3 mol %) in CH₂Cl₂ at room temperature provided APR-03 (Constantin, J. M.; Haven, A. C.; Sarett, L. H. J. Am. Chem. Soc. 1953, 75, 1716-1718) in 68% yield.

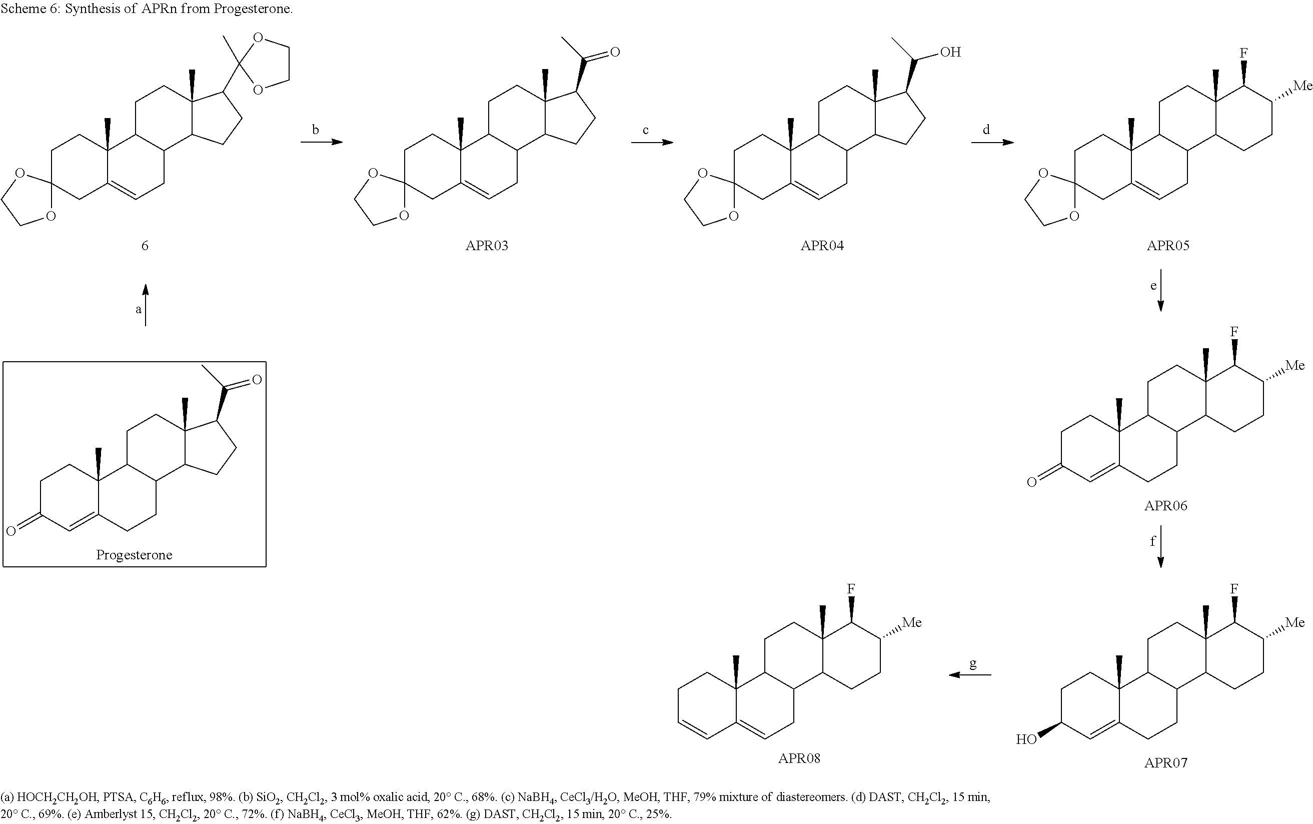

The C20 keto function was then treated with NaBH₄ and CeCl₃ in THF/MeOH, producing the ethylene ketal APR-04. An attempt fluorination of the hydroxy group at the C20 position using DAST as a reagent did not provide the corresponding fluorinated compound; instead, it has been found that the reaction selectively led the rearrangement to the six-membered homosteroid APR-05. Hydrolysis of the ethylene acetal gave APR-06, and reduction of the C3 keto function provided APR-07. It should be noted that an attempt fluorination of the allylic alcohol function using DAST resulted in elimination reaction producing diene steroid APR-08.

### Synthesis of 3,20-Bis-ethylenedioxo-5-pregnene (6)

This compound is prepared according to the protocol of Brown, J. J.; Lenhard, R. N.; Bernstein, S. J. Am. Chem. Soc. 1964, 86, 2183.

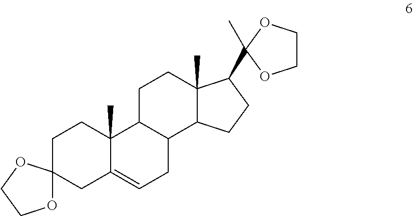 [SMILES: CC1([C@H]2CCC3C4CC=C5CC6(CC[C@]5(C)C4CC[C@@]32C)OCCO6)OCCO1]

A stirred mixture of progesterone (1.0 g, 3.18 mmol) and p-toluenesulfonic acif hydrate (0.06 equiv, 0.19 mmol, 36.3 mg) in toluene (56 mL) and ethylene glycol (10.4 equiv, 33.0 mmol, 1.85 mL) was boiled for 16 h, a water separator being employed. The cooled mixture was then diluted with Et₂O (220 mL), poured into NaHCO₃ solution and the organic layer was washed with water, dried and evaporated. The amorphous solid obtained was purified by flash chromatography (Cyclohexane/Ethyl acetate=3/7) to give 6 in 94% yield as a white solid. R_{f}=0.63 (Cyclohexane/Ethyl acetate=3/7); m.p.=105° C. IR (cm⁻¹): 2930, 1479, 1365, 1261, 1136, 1096, 1047, 946, 866. RMN ¹H (300 MHz) δ ppm: 0.70 (s, 3H, CH₃), 0.95 (s, 3H, CH₃), 1.20 (s, 3H, CH₃), 0.70-2.60 (m, 29H), 3.8 (m, 8H), 5.25 (s, 1H). RMN ¹³C (75 MHz) δ ppm: 140.2, 122.13, 112.0, 109.5, 65.2, 64.6, 64.5, 64.3, 63.3, 58.2, 49.7, 41.8, 40.5, 39.4, 36.7, 36.4, 35.0, 32.4, 30.0, 24.6, 23.8, 23.0, 20.9, 18.9, 12.9.

### Example 39

### Synthesis of 3-Ethylenedioxo-5-pregnene-17-one (APR-03)

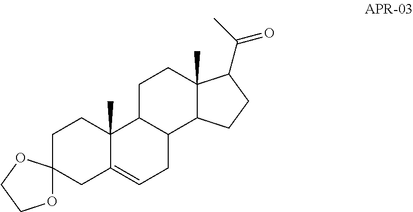 [SMILES: CC(=O)C1CCC2C3CC=C4CC5(CC[C@]4(C)C3CC[C@]12C)OCCO5]

This compound is prepared according to the protocol of Sondheimer, F.; Velasco, M.; Rosenkranz, G. J. Am. Chem. Soc. 1955, 77, 192-194.

Silica gel (4.5 g; 9.0 g SiO₂ per g of acetal) was added with continuous magnetic stirring to a CH₂Cl₂ (6 mL) and an aqueous solution of 3% oxalic acid (0.45 g, 10% of silica gel). After few minutes, the water phase disappears due to adsorption on the silica gel surface. The acetal 6 (500 mg) was added and stirring was continued at room temperature for 1 h. The solid phase was separated by filtration and the solid was washed several times with CH₂Cl₂. The organic layer was washed with an aqueous saturated sodium bicarbonate solution and saturated brine solution and dried over anhydrous sodium sulfate. The extracts were then concentrated and the residue chromatographed on a silica gel column (Cyclohexane/Ethyl acetate=8/2) to give 68% yield of (APR-03). R_{f}=0.35 (Cyclohexane/Ethyl acetate=8/2); m.p.=176° C. IR (cm⁻¹): 2936, 1706, 1424, 1357, 1091, 1026, 953, 910, 869, 731. RMN ¹H (400 MHz) δ ppm: 0.63 (s, 3H, CH₃), 1.03 (s, 3H, CH₃), 2.12 (s, 3H, CH₃), 1.03-2.50 (m, 26H), 3.98 (m, 4H), 5.35 (s, 1H). RMN ¹³C (100 MHz) δ ppm: 209.7, 140.3, 122.0, 109.6, 64.6, 64.4, 63.8, 57.1, 49.7, 44.2, 41.9, 39.0, 36.8, 36.5, 32.0, 31.8, 31.7, 31.2, 24.6, 23.0, 21.2, 19.0, 13.4.

### Example 40

### Synthesis of 3-Ethylenedioxo-20-hydroxy-5-pregnene (APR-04)

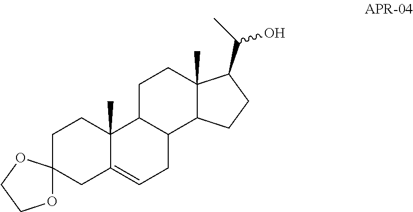 [SMILES: CC(O)[C@H]1CCC2C3CC=C4CC5(CC[C@]4(C)C3CC[C@@]21C)OCCO5]

To a solution of (APR-03) (200 mg, 0.56 mmol) in MeOH (12 mL) and THF (5 mL) was added NaBH₄ (42 mg, 1.12 mmol) and CeCl₃.7H₂O (209 mg, 0.56 mmol) at room temperature. After 1 h, the mixture was quenched with ethyl acetate and was washed with an aqueous solution of 10% HCl. The organic layer was washed with an aqueous saturated sodium bicarbonate solution and saturated brine solution and dried over anhydrous sodium sulfate. The extracts were then concentrated and the residue (1/1 mixture of two epimers at the C20 atom) was purified on a silica gel chromatography column (Cyclohexane/Ethyl acetate: 7/3) to yield 45% of one pure epimer of (APR-04). R_{f1}=0.50 (Cyclohexane/Ethyl acetate: 7/3); m.p.=198-199° C. IR (cm⁻¹)=3515, 2935, 2874, 1447, 1367, 1247, 1095, 1034, 945, 864. RMN ¹H (300 MHz) δ ppm: 0.79 (s, 3H, CH₃), 1.09 (s, 3H, CH₃), 1.19 (d, 3H, CH₃, ³J=4.0 Hz), 1.09-2.70 (m, 26H), 3.73 (m, 1H, CH), 3.95 (m, 4H), 5.34 (s, 1H, CH). RMN ¹³C (100 MHz) δ ppm: 140.4, 122.2, 109.6, 70.7, 64.6, 64.4, 58.6, 56.4, 49.9, 42.4, 42.0, 40.0, 36.8, 31.9, 31.9, 31.2, 25.8, 24.7, 23.8, 21.1, 19.0, 12.5.

### Example 41

### Synthesis of 17aβ-Fluoro-17α-methyl-D-homo-3-ethylenedioxo-5-androstene (APR-05)

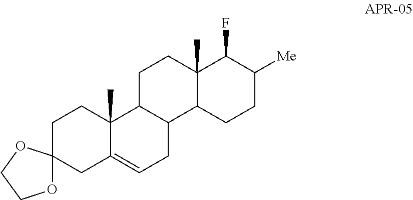 [SMILES: CC1CCC2C3CC=C4CC5(CC[C@]4(C)C3CC[C@]2(C)[C@H]1F)OCCO5]

At −78° 0, the DAST (300 μL, 1.75 mmol) was added to a solution of (APR-04) (282.2 mg, 0.80 mmol) in dry dichloromethane (15 mL), and the solution was stirred at room temperature for 15 min under argon. The reaction was poured into ice water and extracted with CH₂Cl₂. The organic layer was washed with an aqueous saturated sodium bicarbonate solution and saturated brine solution and dried over anhydrous sodium sulfate. The extracts were then concentrated and the residue was purified on a silica gel column chromatography to afford (APR-05) as a single diastereoisomere (Yield=69%). R_{f}=0.70 (Cyclohexane/Ethyl acetate: 7/3); m.p.=146-147° C. IR (cm⁻¹): 2943, 1455, 1367, 1264, 1142, 1099, 1084, 999, 980, 954, 907, 874, 819. RMN ¹H (300 MHz) δ ppm: 0.79 (s, 3H, CH₃), 0.90 (d, 3H, CH₃, ³J=6.3 Hz), 0.91 (s, 3H, CH₃), 0.50-2.60 (m, 29H), 3.55 (dd, 1H, ³J_{H—F}=10.3 Hz, ²J_{H—F}=49.2 Hz), 3.87 (m, 4H), 5.30 (s, 1H). RMN ¹³C (75 MHz) δ ppm: 139.9, 121.9, 109.4, 106.0, (CH, d, J_{C—F}=180 Hz), 64.5, 64.3, 49.7 (CH, d, ³J=5.3 Hz), 49.1, 38.4 (Cq, d, ²J_{C—F}=16.5 Hz), 36.7, 36.0, 32.8, 32.7 (CH, d, ²J=18.0 Hz), 32.3, 32.1 (CH, d, ²J=18.0 Hz), 31.9, 31.0, 27.0, 23.4, 19.6, 18.8, 18.6, 11.8. RMN ¹⁹F δ ppm: −194.9 (td, 1F, J=47.0 Hz, J=8.0 Hz). MS (APCI): m/z=363.3 [M+H]⁺.

### Example 42

### Synthesis of 17aβ-Fluoro-17α-methyl-D-homo-pregn-4-en-3-one (APR-06)

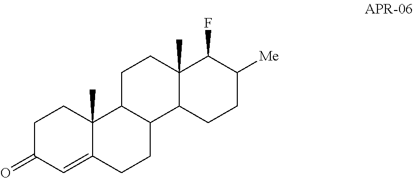 [SMILES: CC1CCC2C3CCC4=CC(=O)CC[C@]4(C)C3CC[C@]2(C)[C@H]1F]

To a solution of (APR-05) (200 mg, 0.63 mmol) in CH₂Cl₂ (5 mL) was added at room temperature Amberlyst (970 mg) and stirring was continued for 2 h. The solid phase was separated by filtration and the solid was washed several times with CH₂Cl₂. The organic layer was washed with an aqueous saturated sodium bicarbonate solution and saturated brine solution and dried over anhydrous sodium sulfate. The extracts were then concentrated and the residue was purified on a silica gel column chromatography (Cyclohexane/Ethyl acetate=3/7) to yield 72% of (APR-06). R_{f}=0.60 (Cyclohexane/Ethyl acetate: 3/7); m.p.=170-171° C. IR (cm⁻¹)=2943, 1677, 1454, 1367, 1264, 1143, 1085, 999, 980, 954, 873, 819, 925. RMN ¹H (300 MHz) δ ppm: 0.90 (s, 3H, CH₃), 0.97 (d, 3H, CH₃, ³J=6.0 Hz), 1.17 (s, 3H, CH₃), 0.90-2.50 (m, 29H), 3.60 (dd, 1H, ³J=10.0 Hz, ²J=49.2 Hz), 5.72 (s, 1H). RMN ¹³C (75 MHz) δ ppm: 199.5, 171.1, 123.5, 105.8 (d, C—F, J_{C—F}=180 Hz), 53.3, 48.9 (CH, d, J=5.3 Hz), 38.6, 36.8, 35.5, 34.9; 34.0, 32.6, 32.0 (d, CH, ³J=18 Hz), 31.4, 29.7, 26.9, 23.3, 19.8, 18.5, 17.6, 11.8. RMN ¹⁹F δ ppm: −192.3 (td, J=49.0 Hz; J=7.5 Hz). MS (APCI): m/z=319.2 [M+H]⁺.

### Example 43

### Synthesis of 17aβ-Fluoro-17α-methyl-D-homo-pregn-4-en-3-ol (APR-07)

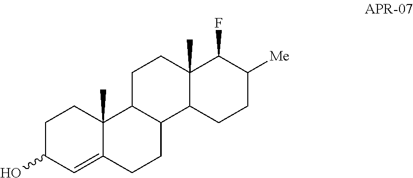 [SMILES: CC1CCC2C3CCC4=CC(O)CC[C@]4(C)C3CC[C@]2(C)[C@H]1F]

(APR-07) was prepared according to the procedure described for (APR-04). The residue was purified on a silica gel chromatography column (Cyclohexane/Ethyl acetate: 3/7) to yield 77% of APR-07 as a mixture of two epimers at the C3 atom. Further careful purification on a silica gel chromatography column (Cyclohexane/Ethyl acetate: 3/7) provided 62% of (APR-07) as a single isomer. R_{f}=0.41 (Cyclohexane/Ethyl acetate: 3/7); m.p.=125-126° C. IR (cm⁻¹)=3256, 2933, 1450, 1377, 1038, 995, 918, 862. RMN ¹H (300 MHz) δ ppm: 0.86 (s, 3H, CH₃), 0.95 (d, 3H, CH₃, J=6.3 Hz), 1.02 (s, 3H, CH₃), 0.50-2.50 (m, 21H), 3.55 (dd, 1H, J_{H—F}=10.2 Hz, J_{H—F}=49.3 Hz), 4.13 (m, 1H, J=15.3 Hz), 5.20 (s, 1H). RMN ¹³C (75 MHz) δ ppm: 147.1, 123.1, 123.1, 106.1 (d, CH, J_{C—F}=180 Hz), 67.8, 53.9, 49.1 (CH, J=4.5 Hz), 38.4, 37.4, 37.0, 35.2, 35.1, 32.6 (CH₂, d, J_{C—F}=9.8 Hz), 32.4, 32.1, 31.9 (d, CH, J=10.5 Hz), 29.5, 19.7, 19.0, 18.5, 11.7. RMN ¹⁹F δ ppm: −194.5 (td, 1F, J=48.9 Hz, J=7.1 Hz). MS (ESI): m/z=343.2 [M+Na]⁺, m/z=663.3 [2M+Na]⁺.

### Example 44

### Synthesis of 17aβ-Fluoro-17α-methyl-D-homo-pregn-3,5-diene (APR-08)

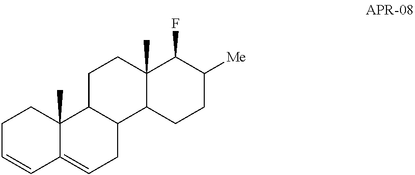 [SMILES: CC1CCC2C3CC=C4C=CCC[C@]4(C)C3CC[C@]2(C)[C@H]1F]

At −78° C., the DAST (360 μL, 1.88 mmol) was added to a solution of (APR-07) (200.0 mg, 0.63 mmol) in dry dichloromethane (12 mL), and the solution was stirred at room temperature for 1 h under argon. The reaction was poured into ice water and extracted with CH₂Cl₂. The organic layer was washed with an aqueous saturated sodium bicarbonate solution and saturated brine solution and dried over anhydrous sodium sulfate. The extracts were then concentrated and the residue was purified on a silica gel column chromatography to afford (APR-08) in a 25% yield. R_{f}=0.91 (Cyclohexane/Ethyl acetate: 7/3). IR (cm⁻¹): 3147, 2900, 1450, 1680, 1043, 995, 918, 862. RMN ¹H (300 MHz) δ ppm: 0.91 (s, 3H, CH₃), 0.93 (s, 3H, CH₃), 0.98 (d, 3H, CH₃, ³J=6.0 Hz), 0.50-2.60 (m, 27H), 3.60 (dd, 1H, J_{H—F}=49.0 Hz), 5.37 (m, 1H), 5.60 (m, 1H), 5.92 (d, 1H, J_{H—F}=12.0 Hz). RMN ¹³C (75 MHz) δ ppm: 141.2, 128.8, 125.4, 122.8, 106.1 (C—F), 50.1, 47.9, 38.7 (Cq, J_{C—F}=16.7 Hz), 36.8, 35.5, 33.5, 32.9, 32.3 (CH, J_{C—F}=17.7 Hz), 32.0, 31.2, 23.4, 23.1, 19.7, 18.8, 18.7, 12.0. MS (APCI): m/z=302.2 [M]⁺, m/z=283.2 [M—F]⁺.

G—Synthesis of antagonists progesterone receptor (APR) starting from 19-nortestosterone:

Readily available 19-nortestosterone has been used as starting material for the synthesis of 19-nor APRn having or not at the C3-position a fluorine substituent (Scheme 7).

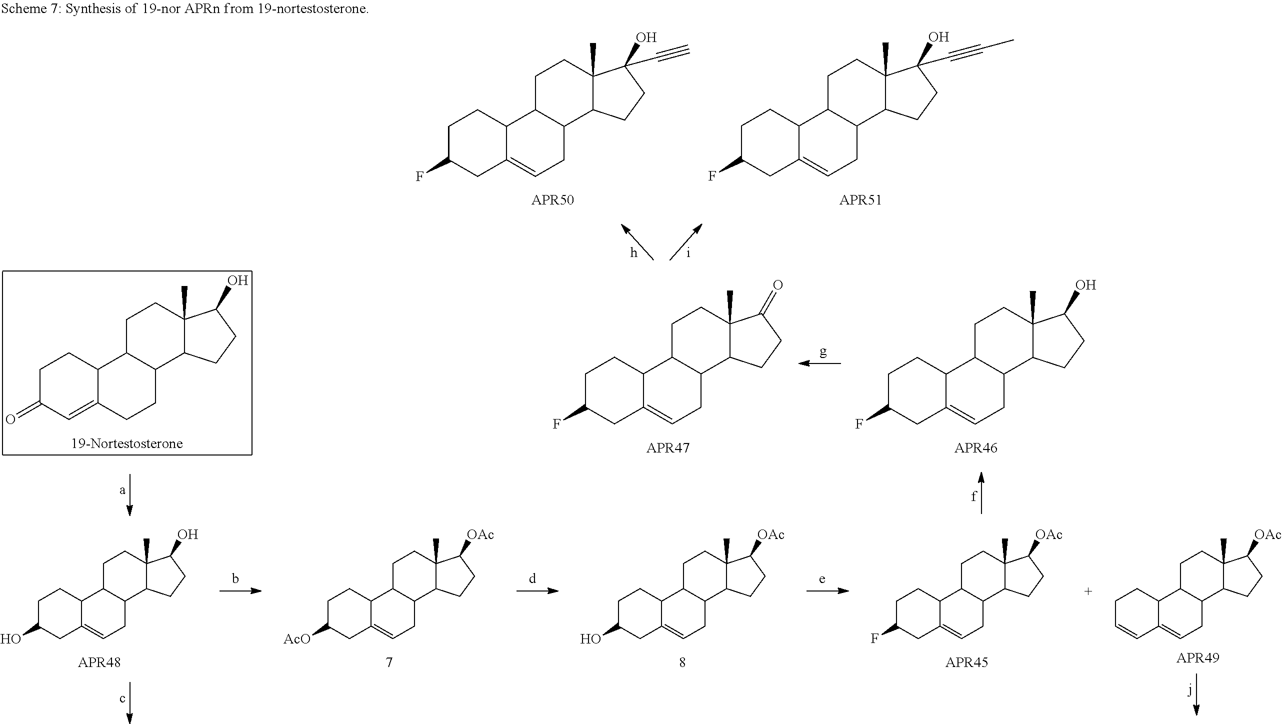

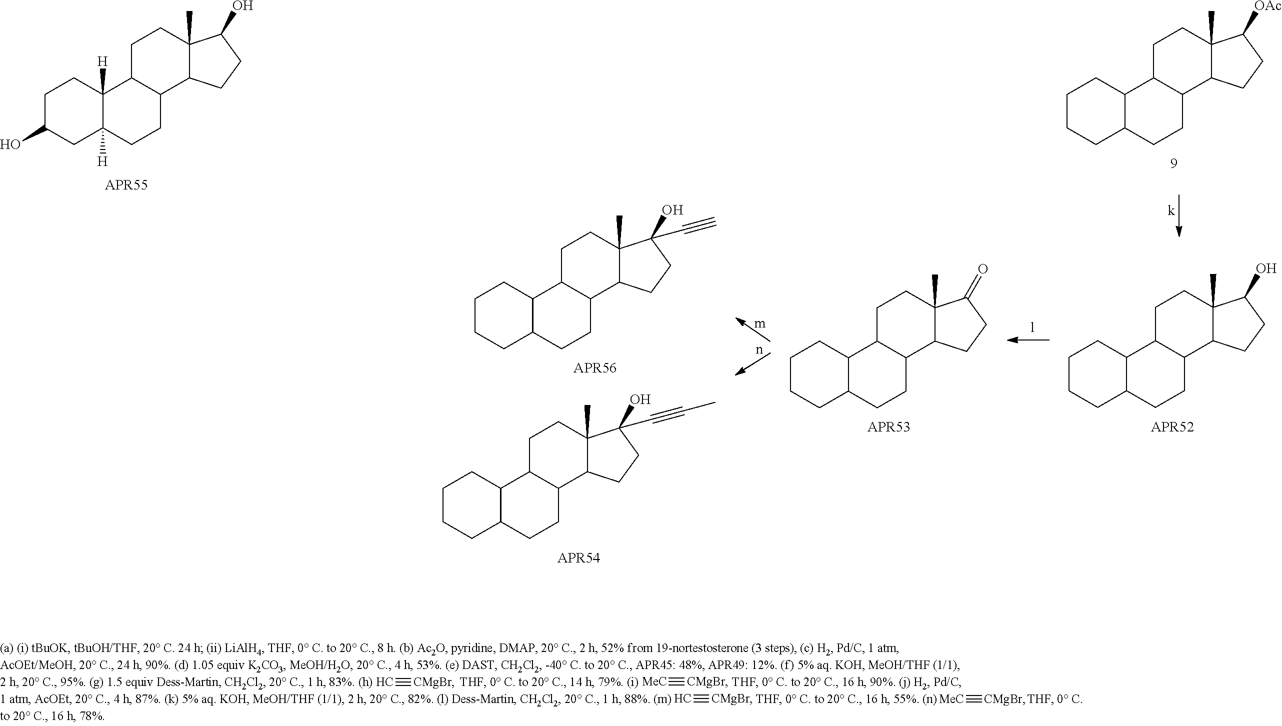

In the first step, the 4,5-double bond of 19-nortestosterone was first deconjugated with tBuOK to form the 5,6-double bond, and the 3-ketone was reduced with LiAlH₄ to avoid reconjugation of the double bond. The resulting diol APR-48, was then either reduced to give APR-55 or acetylated in standard conditions to afford the diacetylated steroid 7. Selective C3-deacetylation followed by fluorination of the resulting alcohol furnished 3β-fluorinated APR-45 together with elimination product APR-49. Starting from APR-45, it was possible to form acetylenic alcohols APR-50 and APR51 in a three step-sequence, involving deacetylation of APR-45 (APR-46), alcohol oxidation (APR-47), and carbonyl condensation with acetylenemagnesium bromide.

The synthesis of 19-nor APRn analogues with no substituent at the A-ring began with the reduction of 3,5-diene system in APR-49. Catalytic hydrogenation using Pd/C and subsequent deacetylation gave APR-52 which was then oxidized with Dess-Martin reagent to obtain ketone APR-53. Further condensation with metal acetylide provided acetylenic alcohols APR-54 and APR-56.

### Example 45

### Synthesis of 19-nor-Androst-5-en-3β,17β-diol (APR-48)

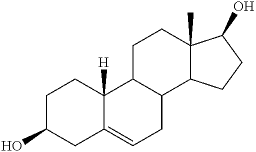 [SMILES: C[C@]12CCC3C(CC=C4C[C@@H](O)CC[C@@H]43)C1CC[C@@H]2O]

This compound is a byproduct (182 mg, 18% yield) in the synthesis of (8). R_{f}=0.16 (eluant: petroleum ether/EtOAc, 80/20). White solid. Mp 161-162° C. ¹H NMR (CD₃OD, 300 MHz): δ 0.72 (s, 3H, Me-18), 0.78-1.60 (m, 12H), 1.77-1.98 (m, 6H) 2.03-2.10 (m, 1H), 2.40 (m, 1H), 3.36 (m, 1H, H-3α), 3.54 (t, 1H, J=8.5 Hz, H-17α), 5.40 (m, 1H, H-6). ¹³C NMR (CD₃OD, 75 MHz): δ 11.6, 24.2, 28.1, 30.7, 31.5, 31.7, 36.2, 37.9, 38.0, 44.2, 44.3, 45.8, 47.2, 51.8, 71.9, 82.6, 122.2, 138.8. MS (APCI+) m/z 277.0 (M+H)⁺, 259.0 [M−(H₂O)+H]⁺.

### Example 46

### Synthesis of 19-nor-5-Androstan-3β,17β-diol (APR-55)

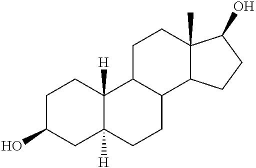 [SMILES: C[C@]12CCC3C(CC[C@H]4C[C@@H](O)CC[C@H]34)C1CC[C@@H]2O]

The same procedure for the synthesis of (5) was followed. From (APR-48), the compound (APR-55) was obtained in 90% yield as a mixture (8:2, α:β). White solid. IR (v cm⁻¹): 1065, 1418, 1558, 2056, 2166, 2359, 2847, 2912, 3360. ¹H NMR (CD₃OD, 300 MHz): δ 0.76 (s, 3H, Me-18), 0.55-2.05 (m, 25H), 3.43-3.67 (m, 2H, H-3, H-17). ¹³C NMR (CD₃OD, 75 MHz): δ 11.7, 24.2, 26.9, 29.7, 30.7, 31.8, 34.9, 36.6, 38.1, 42.7, 42.8, 44.3, 47.9, 49.7, 51.6, 71.2, 82.7. MS (APCI+) m/z 261.0 [M—(H₂O)+H]⁺, 243.0 [M−(2H₂O)+H]⁺.

### 3β,17-Diacetoxy-19-nor-androst-5-ene (7)

(Cadot, C.; Poirier, D.; Philip, A. Tetrahedron 2006, 62, 4384)

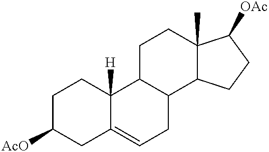 [SMILES: CC(=O)O[C@H]1CC[C@H]2C(=CCC3C2CC[C@@]2(C)C3CC[C@@H]2OC(C)=O)C1]

A mixture of 19-nortestosterone (2 g, 7.28 mmol) and t-BuOK (4 g, 35.65 mmol) in t-BuOH (40 mL) and THF (50 mL) was stirred under nitrogen for 24 h at Room temperature and then quenched by the rapid addition of 10% aq AcOH to the resulting slurry. Saturated aq NaHCO₃ was added and the product was isolated by an extraction with diethyl ether. The combined organic layer was washed with excess aq NaHCO₃, dried over MgSO₄, and evaporated. The crude unconjugated ketone was added to a stirred solution of LiAlH₄ (600 mg, 15.81 mmol) in dry THF (50 mL) at 0° C. After being stirred at 0° C. for 8 h, the reaction mixture was quenched with saturated aq NH₄Cl and extracted with EtOAc. The combined organic layer was washed with brine, dried over MgSO₄, and evaporated. To a stirred solution of crude diol in CH₂Cl₂ (20 mL) were added acetic anhydride (10 mL), pyridine (5 mL) and a catalytic amount of DMAP. The reaction mixture was stirred under nitrogen for 3 h at room temperature, poured into ice cold aq 1 M HCl, and extracted with EtOAc. The combined organic layer was washed with saturated aq NaHCO₃, dried over MgSO₄, and evaporated. The crude product was purified by chromatography (eluant: cyclohexane/EtOAc, 95/05) to afford (7) (1.34 g, 52% yield) as a white solid. R_{f}=0.59 (eluant: cyclohexane/EtOAc, 80/20). Mp 119-120° C. IR (v cm⁻¹): 916, 1029, 1236, 1371, 1436, 1731, 2361, 2940. ¹H NMR (CDCl₃, 300 MHz): δ 0.80 (s, 3H, Me-18), 2.02 (s, 3H, OAc), 2.03 (s, 3H, OAc), 0.78-2.24 (m, 19H), 2.51 (m, 1H), 4.55-4.65 (m, 2H, H-3, H-17), 5.48 (m, 1H, H-6). ¹³C NMR (CDCl₃, 75 MHz): δ 12.1, 21.3, 21.6, 23.5, 26.8, 27.6, 30.2, 30.4, 31.7, 36.4, 36.8, 41.0, 42.8, 42.9, 45.4, 50.2, 73.4, 83.0, 122.4, 136.4, 170.7, 171.4. MS (ESI+) m/z 383.0 (M+Na)⁺.

### 17β-Acetoxy-19-nor-androst-5-en-3β-ol (8)

(Slavikova, B.; Kohout, L.; Budesinsky, M.; Swaczynova, J.; Kasal, A. J. Med. Chem. 2008, 51, 3979)

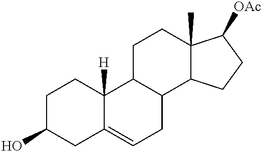 [SMILES: CC(=O)O[C@H]1CCC2C3CC=C4C[C@@H](O)CC[C@@H]4C3CC[C@@]21C]

A solution of potassium carbonate (524 mg, 3.79 mmol) in water (10 mL) and methanol (20 mL) was added to a solution of diacetate (7) (1.3 g, 3.61 mmol) in methanol (120 mL). The mixture was stirred 4 h at room temperature. The saturated aq NH₄Cl was added and the solution was concentrated in vacuo. Brine precipitated a white solid, which was extracted with EtOAc. The extract was washed with brine, dried over MgSO₄, and concentrated in vacuo. Chromatography of the remainder on a column of silica gel (eluant: petroleum ether/EtOAc, 90/10) yielded alcohol (8) (611 mg, 53%) as a white solid. R_{f}=0.19 (eluant: cyclohexane/EtOAc, 80/20). Mp 86-87° C. IR (v cm⁻¹): 1048, 1245, 1373, 1737, 2340, 2362, 2926. ¹H NMR (CDCl₃, 300 MHz): δ 0.81 (s, 3H, Me-18), 2.04 (s, 3H, OAc), 0.83-2.22 (m, 20H), 2.49 (m, 1H), 3.53 (m, 1H, H-3), 4.60 (m, 1H, H-17), 5.45 (m, 1H, H-6). ¹³C NMR (CDCl₃, 75 MHz): δ 12.1, 21.3, 23.5, 26.8, 27.6, 30.4, 30.5, 35.5, 36.5, 36.8, 42.8, 42.9, 45.0, 45.6, 50.2, 71.3, 83.0, 121.5, 137.5, 171.4. MS (ESI+) m/z 341.0 (M+H)⁺.

### Example 47

### Synthesis of 17β-Acetoxy-3β-fluoro-19-nor-androst-5-ene (APR-45)

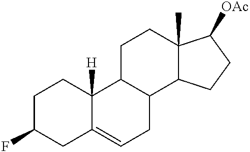 [SMILES: CC(=O)O[C@H]1CCC2C3CC=C4C[C@@H](F)CC[C@@H]4C3CC[C@@]21C]

The same procedure for the synthesis of (APR-16) was followed. From (8), the compound (APR-45) was obtained after chromatography on silica gel (eluant: petroleum ether/EtOAc, 99/01) in 48% yield as a white solid. R_{f}=0.68 (eluant: petroleum ether/EtOAc, 50/50). Mp 104-105° C. IR (v cm⁻¹): 853, 957, 1018, 1245, 1379, 1448, 1732, 2856, 2920. ¹H NMR (CDCl₃, 300 MHz): δ 0.81 (s, 3H, Me-18), 0.74-2.01 (m, 15H), 2.04 (s, 3H, OAc), 2.07-2.25 (m, 4H), 2.59-2.67 (m, 1H), 4.38 (dm, 1H, J_{HF}=50.3 Hz, Hα-3), 4.60 (m, 1H, H-17), 5.50 (m, 1H, H-6). ¹³C NMR (CDCl₃, 75 MHz): δ 12.1, 21.3, 23.5, 26.8, 27.6, 29.5 (d, ³J_{CF}=11.0 Hz), 30.5, 32.6 (d, ²J_{CF}=17.6 Hz), 36.4, 36.8, 42.2 (d, ²J_{CF}=19.4 Hz), 42.7, 42.8, 45.4, 50.2, 82.9, 92.2 (d, ¹J_{CF}=174.0 Hz), 122.8, 136.0 (d, ³J_{CF}=13.1 Hz), 171.4. MS (ESI+) m/z 343.0 (M+Na)⁺, 663.0 (2M+Na)⁺.

### Example 48

### Synthesis of 17β-Acetoxy-19-nor-androst-3,5-diene (APR-49)

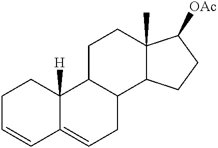 [SMILES: CC(=O)O[C@H]1CCC2C3CC=C4C=CCC[C@@H]4C3CC[C@@]21C]

This compound is a byproduct (70 mg, 12% yield) in the synthesis of (APR-45). R_{f}=0.74 (eluant: petroleum ether/EtOAc, 80/20). White solid. Mp 94-95° C. IR (v cm⁻¹): 845, 1030, 1242, 1372, 1434, 1736, 2361, 2915. ¹H NMR (CDCl₃, 300 MHz): δ 0.82 (s, 3H, Me-18), 0.79-1.95 (m, 13H), 2.03 (m, 1H), 2.04 (s, 3H, Me-21), 2.09-2.19 (m, 4H), 4.63 (m, 1H, H-17), 5.46 (m, 1H, H-6), 5.67 (m, 1H, H-3), 5.99 (d, 1H, J=9.6 Hz, H-4). ¹³C NMR (CDCl₃, 75 MHz): δ 12.1, 21.3, 23.5, 26.2, 26.3, 27.5, 27.7, 31.0, 36.8, 36.9, 41.7, 42.8, 44.1, 50.6, 83.0, 122.9, 127.2, 129.7, 137.0, 171.4. MS (ESI+) m/z 323.0 (M+Na)⁺, 623.0 (2M+Na)⁺.

### 17β-Acetoxy-19-nor-5-androstane (9)

(Hartman, J. A. J. Am. Chem. Soc. 1955, 77, 5151)

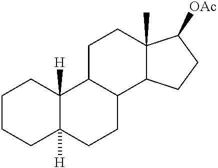 [SMILES: CC(=O)O[C@H]1CCC2C3CC[C@H]4CCCC[C@@H]4C3CC[C@@]21C]

The same procedure for the synthesis of (5) was followed. From (APR-49)(see example 42 above), the compound (9) was obtained after chromatography on silica gel (eluant: petroleum ether/EtOAc, 95/05) in 87% yield as a mixture (8:2, α:β). White solid. R_{f}=0.76 (eluant: petroleum ether/EtOAc, 80/20). IR (v cm⁻¹): 1019, 1241, 1371, 1442, 1728, 2915. From the ¹H NMR and ¹³C NMR data this product was determined to be 84:16 mixture of epimers at C₅, the ¹H NMR (CDCl₃, 300 MHz) for the major α-isomer were δ 0.79 (s, 3H, Me-18), 0.56-1.90 (m, 24H), 2.03 (s, 3H, OAc), 2.08-2.21 (m, 1H), 4.59 (m, 1H, H-17). The ¹³C NMR (CD₃OD, 75 MHz) for the major α-isomer were δ 12.2, 21.3, 23.5, 25.3, 26.6, 27.0, 27.7, 30.4, 30.8, 34.1, 34.7, 37.1, 41.3, 42.9, 43.3, 47.6, 48.6, 50.2, 83.2, 171.4. MS (APCI+) m/z 245.0 [M−(AcOH)+H]⁺.

### Example 49

### Synthesis of 19-nor-5-Androstan-17β-ol (APR-52)

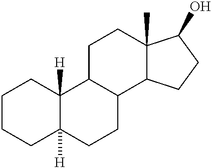 [SMILES: C[C@]12CCC3C(CC[C@H]4CCCC[C@H]34)C1CC[C@@H]2O]

The same procedure for the synthesis of (2) was followed. From (9), the compound (APR-52) was obtained after chromatography on silica gel (eluant: petroleum ether/EtOAc, 90/10) in 82% yield as a mixture (8:2, α:β). White solid. R_{f}=0.36 (eluant: petroleum ether/EtOAc, 80/20). IR (v cm⁻¹): 733, 908, 1053, 1129, 1446, 2362, 2847, 2914, 3300. From the ¹H NMR and ¹³C NMR data this product was determined to be 84:16 mixture of epimers at C₅, the ¹H NMR (CDCl₃, 300 MHz) for the major α-isomer were δ 0.74 (s, 3H, Me-18), 0.58-2.11 (m, 26H), 3.63 (m, 1H, H-17). The ¹³C NMR (CD₃OD, 75 MHz) for the major α-isomer were δ 11.2, 23.4, 25.5, 26.6, 27.0, 30.5, 30.7, 30.8, 34.1, 34.7, 37.0, 41.6, 43.2, 43.4, 47.6, 48.8, 50.5, 82.3. ¹H and ¹³C data were in agreement with the published data (Modica, Colombo, E.; D.; Compostella, F.; Scala, A.; Ronchetti, F. Steroids 2002, 67, 145). MS (ESI+) m/z 285.0 (M+Na)⁺, 547.0 (2M+Na)⁺.

### Example 50

### Synthesis of 19-nor-5-Androstan-17-one (APR-53)

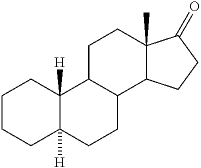 [SMILES: C[C@]12CCC3C(CC[C@H]4CCCC[C@H]34)C1CCC2=O]

The same procedure for the synthesis of (APR-47) was followed. From (APR-52), the compound (APR-53) was obtained after chromatography on silica gel (eluant: petroleum ether/EtOAc, 95/05) in 88% yield as a mixture (8:2, α:β). White solid. R_{f}−0.50 (eluant: petroleum ether/EtOAc, 80/20). IR (v cm⁻¹): 1055, 1247, 1448, 1740, 2332, 2362, 2852, 2915. From the ¹H NMR and ¹³C NMR data this product was determined to be 84:16 mixture of epimers at C₅, the ¹H NMR (CDCl₃, 300 MHz) for the major α-isomer were δ 0.60-0.76 (m, 3H), 0.86 (s, 3H, Me-18), 0.89-2.12 (m, 21H), 2.43 (m, 1H). The ¹³C NMR (CD₃OD, 75 MHz) for the major α-isomer were δ 14.0, 21.8, 25.1, 26.5, 26.9, 30.1, 30.4, 31.8, 33.9, 34.6, 36.0, 41.0, 43.3, 47.5, 48.1, 48.8, 50.9, 221.8. MS (APCI+) m/z 243.0 [M−(H₂O)+H]⁺.

### Example 51

### Synthesis of 17α-(1-Propynyl)-19-nor-5-androstan-17β-ol (APR-54)

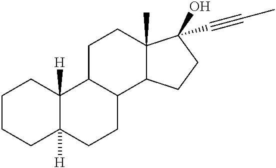 [SMILES: CC#C[C@]1(O)CCC2C3CC[C@H]4CCCC[C@@H]4C3CC[C@@]21C]

The same procedure for the synthesis of (APR-42) was followed. From (APR-53), the compound (APR-52) was obtained after chromatography on silica gel (eluant: petroleum ether/EtOAc, 95/05) in 78% yield as a mixture (8:2, α:β). White solid. R_{f}=0.33 (eluant: petroleum ether/EtOAc, 90/10). IR (v cm⁻¹): 732, 908, 1017, 1129, 1379, 1445, 2361, 2850, 2915, 3377. From the ¹H NMR and ¹³C NMR data this product was determined to be 84:16 mixture of epimers at C₅, the ¹H NMR (CDCl₃, 300 MHz) for the major α-isomer were δ 0.82 (s, 3H, Me-18), 1.86 (s, 3H, Me), 0.63-2.04 (m, 25H), 2.14-2.24 (m, 1H). The ¹³C NMR (CD₃OD, 75 MHz) for the major α-isomer were δ 3.8, 13.0, 23.1, 25.6, 26.6, 27.0, 30.5, 30.8, 33.0, 34.1, 34.7, 39.2, 42.2, 43.4, 47.2, 47.6, 48.3, 49.8, 80.4, 81.6, 83.1. MS (ESI+) m/z 323.0 (M+Na)⁺.

### Example 52

### Synthesis of 17α-Ethynyl-19-nor-5-androstan-17β-ol (APR-56)

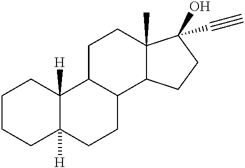 [SMILES: C#C[C@]1(O)CCC2C3CC[C@H]4CCCC[C@@H]4C3CC[C@@]21C]

The same procedure for the synthesis of (APR-42) was followed with ethynylmagnesium bromide. From (APR-53), the compound (APR-56) was obtained after chromatography on silica gel (eluant: petroleum ether/EtOAc, 95/05) in 55% yield as a mixture (8:2, α:β). R_{f}=0.48 (eluant: petroleum ether/EtOAc, 80/20). White solid. IR (v cm⁻¹): 1052, 1130, 1295, 1384, 1447, 1998, 2848, 2915. From the ¹H NMR and ¹³C NMR data this product was determined to be 84:16 mixture of epimers at C₅, the ¹H NMR (CDCl₃, 300 MHz) for the major α-isomer were δ 0.84 (s, 3H, Me-18), 0.63-2.04 (m, 25H), 2.22-2.31 (m, 1H), 2.55 (s, 1H, H_{C≡C}). The ¹³C NMR (CD₃OD, 75 MHz) for the major α-isomer were δ 12.9, 23.1, 25.5, 26.6, 27.0, 30.4, 30.8, 32.9, 34.1, 34.7, 39.1, 42.1, 43.4, 47.1, 47.6, 48.3, 49.9, 73.9, 80.2, 87.9. MS (ESI+) m/z 309.0 (M+Na)⁺

### Example 53

### Synthesis of 31-Fluoro-19-nor-androst-5-en-17β-ol (APR-46)

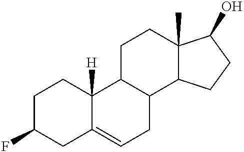 [SMILES: C[C@]12CCC3C(CC=C4C[C@@H](F)CC[C@@H]43)C1CC[C@@H]2O]

The same procedure for the synthesis of (2) was followed. From (APR-45), the compound (APR-46) was obtained after chromatography on silica gel (eluant: petroleum ether/EtOAc, 80/20) in 95% yield as a white solid. R_{f}=0.27 (eluant: petroleum ether/EtOAc, 80/20). Mp 121-122° C. IR (v cm⁻¹): 734, 911, 1021, 1069, 1355, 1447, 2361, 2912, 3283. ¹H NMR (CDCl₃, 300 MHz): δ 0.76 (s, 3H, Me-18), 0.79-2.18 (m, 20H), 2.59-2.67 (m, 1H), 3.65 (m, 1H, H-17), 4.38 (dm, 1H, J_{HF}=50.3 Hz, Hα-3), 5.50 (m, 1H, H-6). ¹³C NMR (CDCl₃, 75 MHz): δ 11.1, 23.4, 26.9, 29.5 (d, ³J_{CF}=11.0 Hz), 30.5, 30.6, 32.6 (d, ²J_{CF}=17.7 Hz), 36.6, 36.7, 42.2 (d, ²J_{CF}=19.3 Hz), 42.7, 43.2, 45.6, 50.4, 82.1, 92.3 (d, ¹J_{CF}=174.1 Hz), 122.9, 136.0 (d, ³J_{CF}=13.0 Hz). MS (ESI−) m/z 277.0 (M−H)⁻.

### Example 54

### Synthesis of 3β-Fluoro-19-nor-androst-5-en-17-one (APR-47)

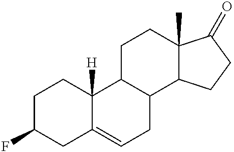 [SMILES: C[C@]12CCC3C(CC=C4C[C@@H](F)CC[C@@H]43)C1CCC2=O]

General Procedure for Oxydation by Dess-Martin

(Corey, E. J.; Huang, A. X. J. Am. Chem. Soc. 1999, 121, 710):

To a stirred solution of (APR-46) (195 mg, 0.75 mmol) in 10 mL of CH₂Cl₂ at room temperature was added the Dess-Martin periodinane (446 mg, 1.05 mmol). The resulting mixture was stirred at room temperature for 1 h. The saturated aqueous NaHCO₃ and 10% aqueous Na₂S₂O₃ were added. The aqueous phase was extracted three times with 10 mL of CH₂Cl₂. The combined CH₂Cl₂ extract was dried over anhydrous Na₂SO4 and passed through a short silica gel column (eluant: petroleum ether/EtOAc, 95/05) to afford (APR-47) (160 mg, 83% yield) as a white solid. R_{f}=0.54 (eluant: petroleum ether/EtOAc, 80/20). Mp 102-103° C. IR (v cm⁻¹): 735, 960, 1015, 1249, 1376, 1452, 1736, 2933. ¹H NMR (CDCl₃, 300 MHz): δ 0.79-0.88 (m, 2H), 0.89 (s, 3H, Me-18), 1.10-2.19 (m, 16H), 2.46 (m, 1H), 2.61-2.69 (m, 1H), 4.39 (dm, 1H, J_{HF}=50.2 Hz, Hα-3), 5.53 (m, 1H, H-6). ¹³C NMR (CDCl₃, 75 MHz): δ 13.8, 21.8, 26.6, 29.5 (d, ³J_{CF}=11.0 Hz), 29.8, 31.5, 32.5 (d, ²J_{CF}=17.7 Hz), 35.9, 36.1, 42.1 (d, ²J_{CF}=19.5 Hz), 42.7, 45.6, 47.9, 50.9, 92.1 (d, ¹J_{CF}=174.2 Hz), 122.5, 136.2 (d, ³J_{CF}=13.0 Hz), 221.2. MS (ESI+) m/z 299.0 (M+Na)⁺.

### Example 55

### Synthesis of 17α-Ethynyl-3β-fluoro-19-nor-androst-5-en-17β-ol (APR-50)

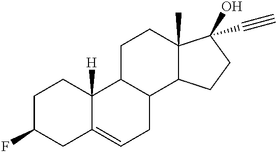 [SMILES: C#C[C@]1(O)CCC2C3CC=C4C[C@@H](F)CC[C@@H]4C3CC[C@@]21C]

The same procedure for the synthesis of (APR-42) was followed with ethynylmagnesium bromide. From (APR-47), the compound (APR-50) was obtained after chromatography on silica gel (eluant: petroleum ether/EtOAc, 95/05) in 79% yield as a white solid. R_{f}−0.42 (eluant: petroleum ether/EtOAc, 80/20). Mp 149-150° C. IR (v cm⁻¹): 731, 962, 1017, 1158, 1379, 1448, 2362, 2932, 3305. ¹H NMR (CDCl₃, 300 MHz): δ 0.87 (s, 3H, Me-18), 0.79-2.34 (m, 22H), 2.56 (s, 1H, H_{C≡C}), 2.64 (m, 1H), 4.39 (dm, 1H, J_{HF}=50.1 Hz, Hα-3), 5.49 (m, 1H, H-6). ¹³C NMR (CDCl₃, 75 MHz): δ 12.7, 23.1, 27.0, 29.5 (d, ³J_{CF}=11.1 Hz), 30.5, 32.6 (d, ²J_{CF}=17.4 Hz), 32.6, 37.2, 39.1, 42.2 (d, ²J_{CF}=19.3 Hz), 42.7, 45.1, 47.0, 49.9, 74.1, 80.1, 92.3 (d, ¹J_{CF}=174.3 Hz), 122.8, 136.0 (d, ³J_{CF}=13.1 Hz). MS (APCI+) m/z 285.0 [M−(H₂O)+H]⁺.

### Example 56

### Synthesis of 3β-Fluoro-17α-(1-propynyl)-19-nor-androst-5-en-17β-ol (APR-51)

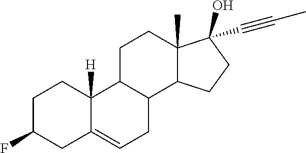 [SMILES: CC#C[C@]1(O)CCC2C3CC=C4C[C@@H](F)CC[C@@H]4C3CC[C@@]21C]

The same procedure for the synthesis of (APR-42) was followed. From (APR-47), the compound (APR-51) was obtained after chromatography on silica gel (eluant: petroleum ether/EtOAc, 95/05) in 90% yield as a white solid. R_{f}=0.46 (eluant: petroleum ether/EtOAc, 80/20). Mp 72-73° C. IR (v cm⁻¹): 736, 853, 1014, 1266, 1378, 1447, 1663, 2356, 2935, 3360. ¹H NMR (CDCl₃, 300 MHz): δ 0.84 (s, 3H, Me-18), 1.86 (s, 3H, Me), 0.77-2.24 (m, 19H), 2.63 (m, 1H), 4.38 (dm, 1H, J_{HF}=50.3 Hz, Hα-3), 5.49 (m, 1H, H-6). ¹³C NMR (CDCl₃, 75 MHz): δ 3.9, 12.9, 23.1, 27.1, 29.5 (d, ³J_{CF}=10.9 Hz), 30.5, 32.6 (d, ²J_{CF}=17.9 Hz), 32.8, 37.3, 39.1, 42.2 (d, ²J_{CF}=19.3 Hz), 42.8, 45.2, 47.1, 49.8, 80.2, 81.8, 82.9, 92.3 (d, ¹J_{CF}=174.1 Hz), 122.9, 136.0 (d, ³J_{CF}=13.0 Hz). MS (APCI+) m/z 299.0 [M−(H₂O)+H]⁺.

Biological Results

Biological Protocols

PR Transactivation Assays in HEK 293T.

HEK 293T cells were routinely cultured in a high-glucose DMEM medium (Invitrogene, Cergy Pontoise, France), 20 mM HEPES, 2 mM glutamine, 1× non essential amino acids, 100 U/mL penicillin and 100 μg/mL streptomycin supplemented with 10% foetal calf serum (FCS) in a humidified atmosphere at 37° C. and with 5% CO₂. One day before transfection, the cells were seeded at 3×10⁶ cells/80 nm diameter culture Petri dish and cultured overnight in the same medium. Six hours before transfection, the FCS supplemented medium was replaced by the same medium supplemented with 10% dextran-charcoal treated FCS. Transfections were carried out using the calcium phosphate precipitation method. The calcium phosphate precipitate was prepared with 0.5 μg pchPRB (Petit-Topin, et al Mol Pharmacol, 2009, 75, 1317), 7 μg reporter vector (GRE2-Luc from A. Biola-Vidamment and M. Pallardy) and 1 μg of pcβgal for an internal transfection control, in 1 mL 140 mM NaCl, 0.75 mM Na₂HPO₄, 25 mM HEPES, 125 mM CaCl₂ pH 7.05 and added to the cells 30 minutes later. After 16 h incubation, the transfected cells were washed with PBS containing 2.5 mM EDTA, trypsinized and pooled. The transfected cells were replated in 24-well plates (100.000 transfected cells/well). 4 h later, APRn molecules (1 μM) were added to the transfected cells in the absence (agonist effect) or presence (antagonist effect) of progesterone (1 nM) and the incubation maintained for 24 h at 37° C. Cells were lysed in 300 μl PBS 1×, 25 mM glycylglycine, 4 mM EDTA, 15% glycerol, 1% triton X-100, 15 mM MgSO₄, pH 7.8 supplemented with 2 mM β-mercaptoethanol. The luciferase activities were quantified by using a Mithras LB940 reader microplates (Berthold).

The APRn “agonist efficacy” was determined from the following formula:

Agonist efficacy=[luciferase activity]₁/[luciferase activity]₂×100

in which the [luciferase activity]₁ and [luciferase activity]₂ are measured in the presence of 1 μM APRn and 1 nM progesterone, respectively.
The APRn “antagonist efficacy” was calculated from the following formula:

Antagonist efficacy=100−[luciferase activity]₁/[luciferase activity]₂×100

in which [luciferase activity]₁ and [luciferase activity]₂ are measured in the presence of 1 μM APRn plus 1 nM progesterone and 1 nM progesterone alone, respectively. The results are the mean±SEM of three to five independent experiments.

Measurement of the APRn Selectivity in HEK 293T.

The HEK-293T cells were cultured and transfected according to the method described above for the PR transactivation assays. The expression vectors pcDNA-hAR, kindly provided by G. A. Coetzee, pchGR (Hellal-Levy, C et al. FEBS Lett 1999, 464, 9), and pchMR (Fagart, J. et al. EMBO J. 1998, 17, 3317) were used for studying the ability of APRn to activate or inactivate the AR, GR and MR, respectively. For each transfection assays, 2 μg of the expression vector was used together with 7 μg of the GRE2-Luc reporter vector. Dihydrotestosterone (DHT), dexamethasone (Dex) and Aldosterone (Aldo) were selected as AR, GR and MR ligands, respectively. They were used at the concentration of 10⁻⁹ M. The APRn agonist and antagonist efficacy for AR, GR and MR were measured as described above for PR. The results are the mean±SEM of three independent experiments.

Establishment of the MDA-MB-231 iPRAB cells.

The PR negative breast cancer cells MDA-MB-231 were routinely maintained in RPMI 1640 medium with L-glutamine enriched with 5% fetal calf serum (FCS) and supplemented with antibiotics (penicillin 100 UI/ml, streptomycin 100 μg/ml). MDA MB-231 iPRAB cells allowing the bi-inducible expression of PRA and PRB isoforms were generated in two steps. First, pZX-TR vector expressing inducer proteins required for Tet-on (Tet-Repressor) and Ecdysone receptor-based system (EcR and RXR hybrids) was engineered using pcDNA6-TR (T-REx system, Invitrogen) and pZRD (Lessard, J. Prostate 2007 67, 808) derived from Rheoswitch system (NE Biolabs). Transfection of MDA-MB-231 cells by pZX-TR (harboring zeocin resistant gene) was performed using Lipofectamine 2000 (Invitrogen). Three hundred clones resistant to Zeocin (1 μg/ml) were isolated, amplified and screened for the proper functioning of both RheoSwitch and Tet-on inducible systems by transient transfection with pGal-4-luciferase or pTO-luciferase reporter genes generated as described. Luciferase activity was determined in the absence or presence of respective inducer ligand RSL1 (diacylhydrazine, a non-steroidal agonist of ecdysone, NE Biolabs) (500 nM) or Doxycycline (Dox, a tetracyclin analog, Sigma-Aldrich) (1 μg/ml) after 24 h. The clone that produced minimal background in the absence of both RSL1 and Dox and high inducible expression level on reporter gene assays by both systems was finally selected, verified and amplified (clone 250). This cell line was then further stably transfected by PR isoforms expressing vectors specifically engineered: pGaluas-PRA (harboring neomycin resistant gene) expressing PRA under the control of Gal4 Upstream Activating Sequences upstream of a truncated CMV promoter, and pTO-PRB (harboring blasticidin resistant gene) expressing PRB under the control of TetO2 responsive elements downstream of the full CMV promoter. Clones resistant to zeocin (1 μg/ml), neomycin (500 μg/ml) and blasticidin (2 μg/ml) were then isolated, amplified and screened by Western blot for PR isoforms expression in the absence or presence of both RSL1 (500 nM) and Dox (1 μg/ml) after 24 h. A clone with undetectable basal PR expression and comparable high inducible expression levels of both PR isoforms in the presence of RSL1 and Dox was finally selected. It was used to evaluate APRn properties and was defined as MDA-MB-231 iPRAB cells conditionally expressing PRB in the presence of doxycyclin (1 μg/ml).

PR Transactivation Assays in MDA-MB-231 iPRAB Cells.

The cells were cultured in 96-well plates in RPMI 1640 medium with L-glutamine without phenol red, enriched with 5% DCC serum containing inducer ligand RSL1 (500 nM) or Dox (1 μg/ml) for PRA or PRB expression during 24 h. Cells were then transfected with GRE2-Luc (100 ng) and β-galactosidase (20 ng) plasmids during 6 h, using Lipofectamine 2000 (Invitrogen). Cells were incubated during 24 h with vehicle or progesterone (1 nM) or APRn molecules (1 μM) alone or in combination to determine PRA or PRB mediated agonistic as well as antagonistic activity on PR reporter gene transcription. Whole cell extracts were collected using lysis buffer (Promega). Luciferase and β-galactosidase activity or total protein contents (BCA assay) were determined using a luminometer (Victor 378, Perkin Elmer). Luciferase activity was normalized by β-galactosidase activity or total protein contents. The results are expressed as percentage of agonistic or antagonistic activity as compared to luciferase activity determined after progesterone treatment.

Mammalian Two Hybrid Assays.

To investigate whether hPR was able to recruit transcriptional corepressors upon APRn binding, two hybrid assays have been performed in HEK 293T cells. Cell transfection were carried out according to the protocol used for the PR transactivation assays, with 1 μg of pGAL4-SMRT or pGAL4-NcoR, 2.5 μg of the PR expression vector pV16-PR and 5 μg of the reporter pG5-luc. The plasmid pGAL4-SMRT and pGAL4-NcoR, provided by P. Balaguer, encode the fusion protein between the GAL4 DNA-binding domain (GAL4DBD) and the receptor interacting domain (RID) of the corepressor NCoR and SMRT, respectively. The expression vector pV16PR encodes the VP16 activation domain of herpes simplex virus fused to the entire hPR and pG5-luc, provided by P. Fuller, encodes the luciferase gene driven by a GAL4-responsive promoter. Two hybrid assays were also performed to check whether PR was able to recruit transcriptional coactivators upon APRn binding and also whether these molecules inhibit the progesterone-induced coactivator recruitment. Cell transfection were carried out according to the PR transactivation assays, by using 2 μg of pM-TIF2/Nter-RID or pM-TIF2/RID, 2 μg of the expression vector pV16PR and 5 μg of the reporter pG5luc. The plasmids pM-TIF2/Nter-RID encodes the fusion proteins between the GAL4 DBD and the 1-867 TIF2 sequence corresponding to the Nter and RID domains. The plasmids pMTIF2-Nter encodes the fusion proteins between the GAL4 DBD and the 1-623 TIF2 sequence corresponding to the Nter domain. After 16 h incubation, the transfected cells were washed with PBS containing 2.5 mM EDTA, trypsinized and replated in 24-well plates. 4 h later, APRn molecules (10⁻⁸-10⁻⁵ M), RU486 or progesterone (10⁻¹⁰−10⁻⁷M)) were added to the cells and the incubation maintained for 24 h at 37° C. Cells were lysed and the luciferase and activities were quantified by using a Mithras reader microplates (Berthold).

Quantitative RT-PCR

MDA-MB-231 iPRAB cells were cultured in 6-well plates in RPMI 1640 medium with L-glutamine without phenol red, enriched with 5% DCC serum containing inducer ligand RSL1 (500 nM) or Dox (1 μg/ml) for PRA or PRB expression during 24 h. Cells were treated during 6 h with vehicle or progesterone (1 nM) or APRn (1 μM) alone or in combination to determine agonistic and antagonistic effects on endogenous gene transcription mediated by PRA or PRB isoforms. Total RNA was then extracted using TRIZOL reagent (Invitrogen). One μg of total RNA was treated with DNase I Amplification Grade (Invitrogen) and then reverse transcribed using cDNA RT kit from Applied Biosystems (Courtaboeuf, France) and random primers. Following a ten-fold dilution, the cDNA samples were amplified in duplicate by real-time PCR in ABI 7300 apparatus (Applied Biosystems), using the Power SYBR Green PCR Master Mix (Applied Biosystems) in the presence of 300 nM of forward and reverse specific primers. A dissociation curve was also obtained at the end of the reaction to verify the specificity of the pair of primers. Standard curve for PCR calibration of each gene transcript tested was obtained with the corresponding amplicon subcloned in pGEMT-easy (Promega) and verified by sequencing analysis. The expression level of each gene transcript was normalized to 18S RNA level, and results were expressed as means of relative concentrations of six samples (attomole of specific gene cDNA/femtomole of 18S cDNA±SEM.)

Inhibition of Progesterone Anti-Estrogenic Effects In Vivo

The anti-progesterone activity of APR19 has been investigated in immature 5-week old B6D2 female mice pre-treated by estrogens to induce endometrium proliferation. Mice (10-12 g) were primed with 25 ng estradiol (E2) IP at day 0. At day 3 to 6, estrogen-primed mice are daily injected with either 50 μg progesterone (P4) alone or in combination with 750 μg of APR-19. The last injection is supplemented with 25 ng of E2 to induce progesterone receptor response. Mice were sacrificed on day 7, and uterine horns were excised and weighed.

Results

Efficacy of APRn to Activate or Inactivate hPRB

All the synthesized APRn were classified in 4 series depending on the nature of their C3 substituent (R₁/R′₁ groups). The first one (series 1) includes APRn with no C3 substituent (R₁═R′₁═H) whereas series 2, 3 and 4 are related to APRn bearing a methoxyl (R₁═OMe), a hydroxyl (R₁═OH) and a fluorine atom (R₁═F), respectively. The agonist/antagonist activities of APRn were determined either in human HEK293T cells transiently expressing hPRB or in human MDA-MB-231 iPRAB cells conditionally expressing hPRB, by using a luciferase reporter gene placed under the control of a glucocorticoid response element (GRE2-Luc). The APRn “agonist efficacy” was determined from the luciferase activity measured in the presence of APRn (1 μM). The APRn “antagonist efficacy” was determined from the luciferase activity measured in the presence of APRn (1 μM) plus progesterone (1 nM). The “agonist efficacy” and “antagonist efficacy” are expressed as defined in the biological protocols section.

APRn Lacking C3-Substituent (Series 1)

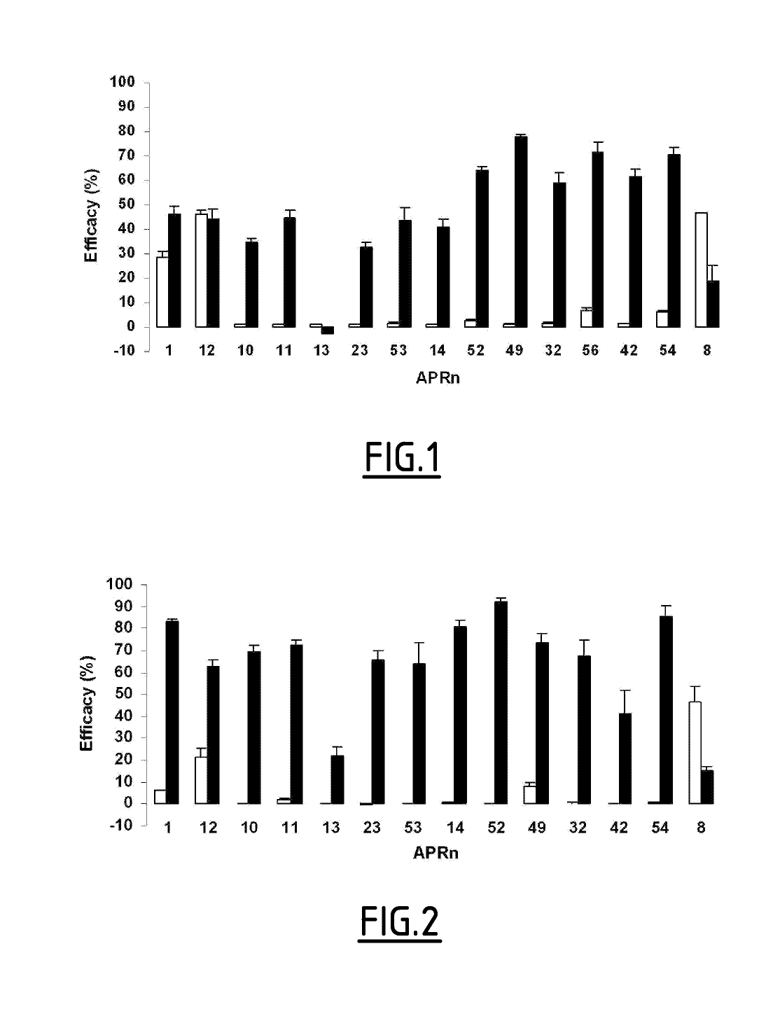

APR-01 (Steraloids, Newport, R.I. USA) which displays a great resemblance with progesterone but having no C3 substituent was the first molecule tested for its hPRB agonist/antagonist activity. As shown in FIG. 1, it displays an antagonist character and has an antagonist efficacy of 48%. Nevertheless, APR-01 is also able to activate hPRB with an efficacy of 30%. Thus, APR-01 is a partial hPRB agonist. Similarly, APR-12 and homosteroid APR-08, behave as partial antagonist.

All the other APRn of series 1 (APR-10, APR-11, APR-13, APR-23, APR-53, APR-14, APR-52, APR-49, APR-32, APR-56, APR-42, APR-54, APR-8) are full antagonists. Their antagonist efficacies are highly dependent on the C17 substituent. The presence of a 17β-hydroxyl (APR-14, APR-52 and APR-49), or a 17β-hydroxyl-17α-alkynyl substituents (APR-32, APR-56, APR-42 and APR-54) increased the antagonist efficacy. Interestingly, the efficacies of the 19-norsteroids are higher than those of the parent molecules (APR52/APR14, APR56/APR32, APR54/APR42).

In the cellular model expressing hPRB in a conditional manner (MDA-MB-231 iPRAB cells), the agonist/antagonist profile of the APRn lacking the C3 substituent was similar (FIG. 2). Nevertheless in the MDA-MB-231 iPRAB cells, the efficacy of the APRn was higher than in the HEK 393T cells, suggesting that the antagonist efficacy of the steroids might depend on the hPRB expression level.

APRn with 3-Methoxy Substituent (Series 2)

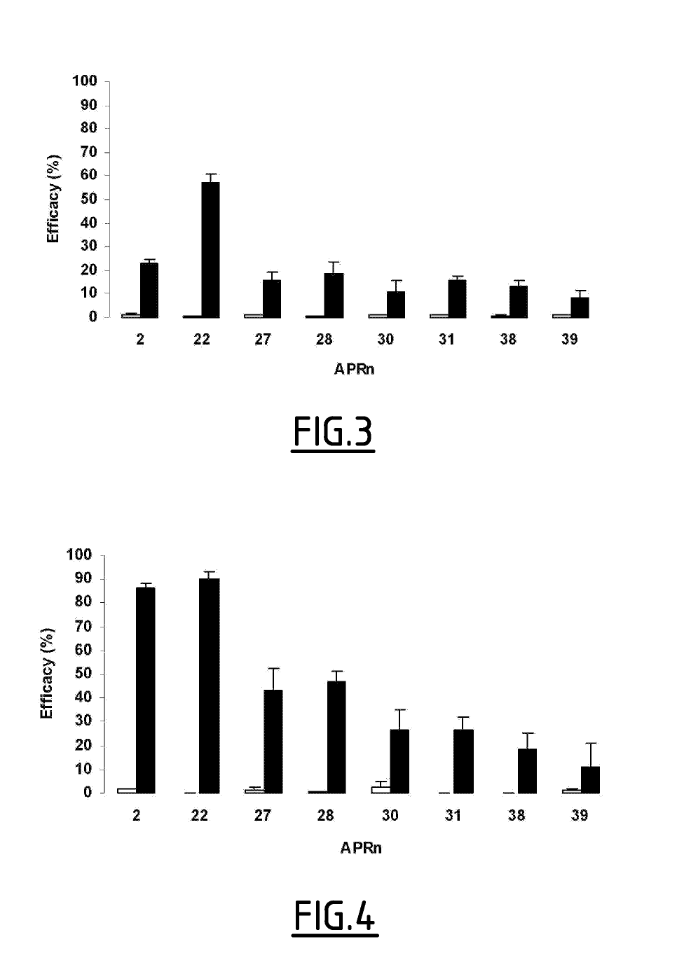

All the APRn (APR2, APR22, APR27, APR28, APR30, APR31, APR38, APR39) characterized by the presence of a 3-methoxy group display an antagonist character without any agonist activity. Again, in the MDA-MB-231 iPRAB cells, the efficacy of the APRn was higher than in the HEK 393T cells. In both cellular models (FIGS. 3 and 4), APR02 and APR22 are the most potent molecules, indicating that the presence of a 20-keto group is more favorable than a 20-hydroxyl function. When APR22 (Series 2) and APR1 (Series 1) were compared, one can note that the presence of the C3 methoxy group completely abolishes the agonist character without having any effect on the antagonist efficacy.

APRn with a 3-Hydroxy Substituent (Series 3)

Series 3 comprises APR15, APR20, APR9, APR18, APR29, APR55, APR48 and APR7.

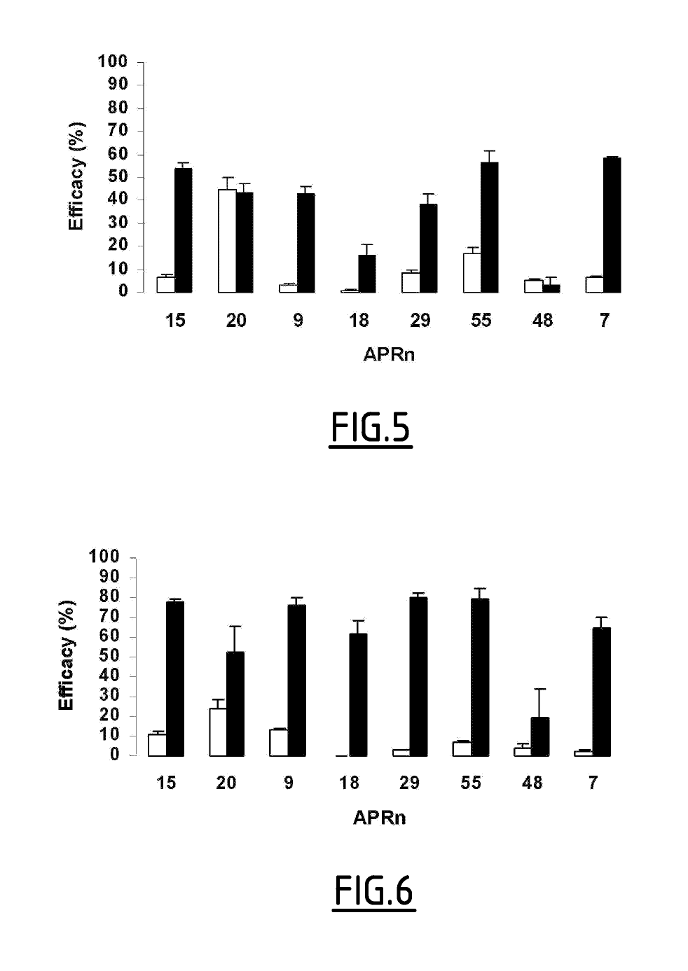

Most of the APRn of series 3 (APR15, APR20, APR29 and APR55) are able to activate hPRB, but their agonist efficacies are low (FIGS. 5 and 6). Except for APR48, the molecules of this series behave as antagonists, and their efficacies are not modulated by the nature of the C17 substituent. In addition, the presence of a Δ^{5,6} insaturation in APR15 decreases the agonist character without altering its antagonist efficacy (compare APR15 and APR20). In contrast, the presence of a Δ^{5,6} insaturation decreases the antagonist efficacy of 20-hydroxylated steroids (compare APR18 and APR29). In both experimental cell lines, the fluorinated homosteroid APR07 appears to be the most potent hPRB antagonist in this series.

APRn with a C3-Fluorine Atom (Series 4)

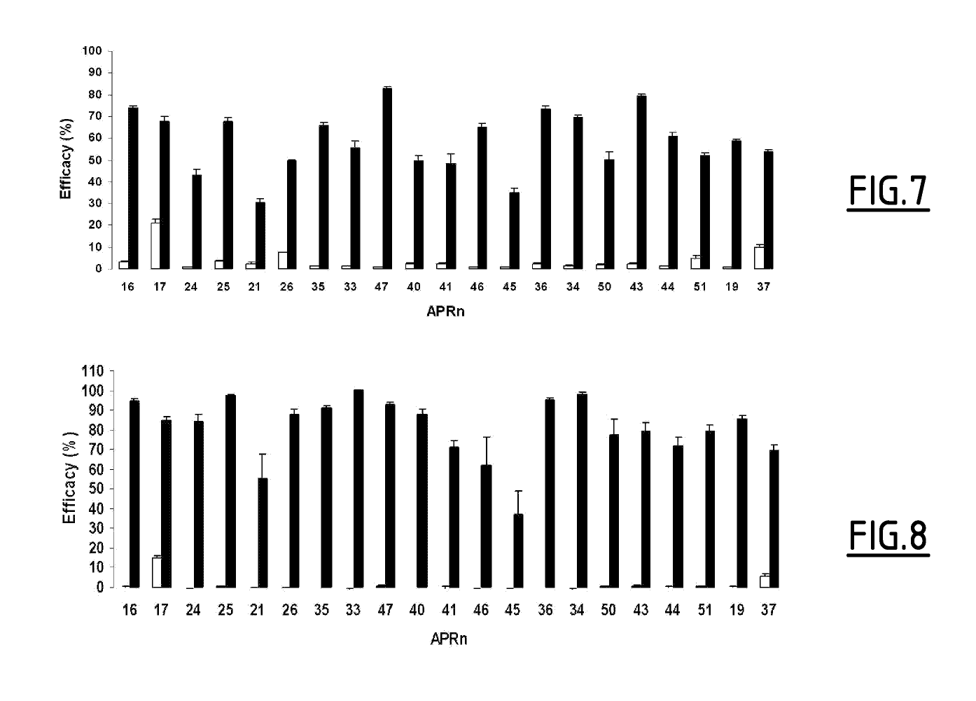

A C3 fluorine atom was introduced with the aim to maximally reduce the agonist activity of the molecules and to prevent their metabolism. All the fluoro-APRn of this series (APR16, APR17, APR24, APR25, APR21, APR26, APR35, APR33, APR47, APR40, APR41, APR46, APR45, APR36, APR34, APR50, APR43, APR44, APR51, APR19, APR37) were found to be efficient hPRB antagonists, whatever the nature of C17 substituent (e.g.; APR16, APR33, APR34, APR44) in both experimental models (FIGS. 7 and 8). Again, in the MDA-MB-231 iPRAB cells, the efficacy of the APRn was higher than in the HEK 393T cells (FIGS. 7 and 8). Interestingly, the presence of a six-membered D-ring (APR19) instead of a five-membered one (APR16) also had no influence on the hPRB antagonist activity. All these findings suggest the dramatic influence of the C3 fluorine atom on the hPRB antagonist activity.

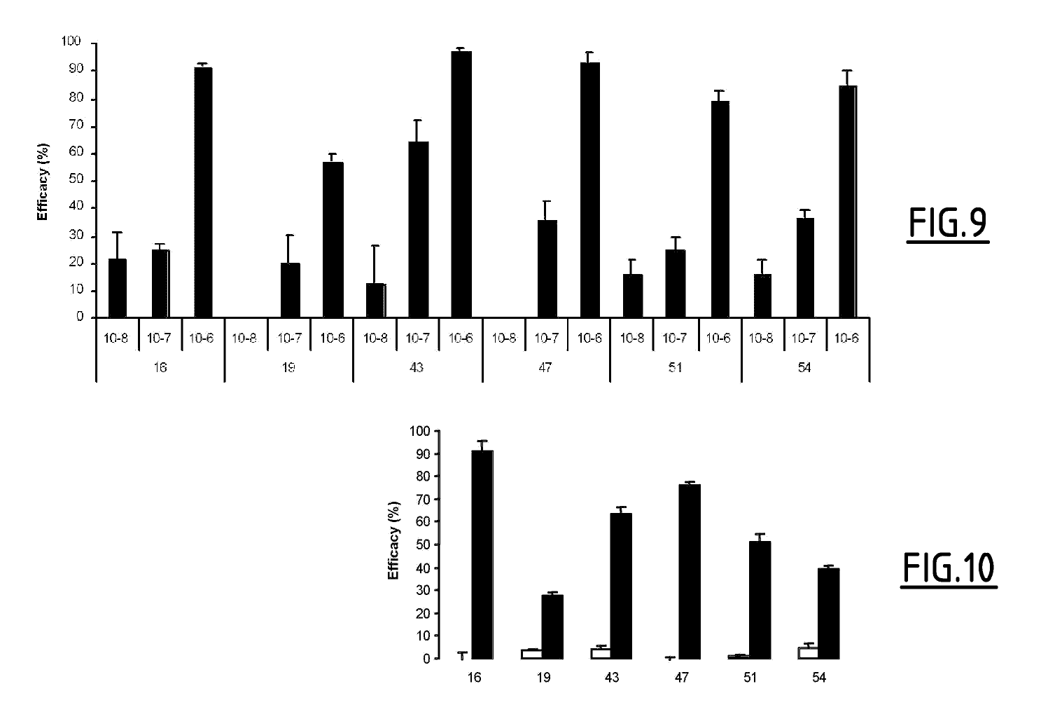

As shown in FIGS. 7 and 8, only APR17 shows a partial agonist activity. It is interesting to point out that the agonist activity of this molecule is completely abolished by further introducing a Δ^{5,6} insaturation (APR16). Interestingly, the presence of a Δ^{5,6} insaturation has nearly no effect on the antagonist efficacy of molecule with a 20-keto group (APR16 vs APR17). Dose response curves presented in FIG. 9 pointed out that the IC₅₀ values of APR16, APR19, APR43, APR47, APR51 and APR54 are about 5×10⁻⁷M.

APRn Efficiency on Endogenous Gene Transcription

The selected APRn (APR16, APR19, APR43, APR47, APR51, APR54) were verified for their agonist and antagonistic properties on endogenous gene transcription. A ligand activated PRB target gene, amphiregulin was selected for these studies (Georgiakaki, M. Mol Endocrinol 2006, 20, 2122) and quantitative RT-PCR analysis was performed in MDA-MB-231 iPRAB cells as described in Materials and Methods. While the selected APRn were nearly unable to exert agonistic effect on amphiregulin gene transcription, their antagonistic properties varied from 30 to 90% (FIG. 10).

Selectivities of APRn Against PR Vs AR, GR and MR

For the most active APRn, their selectivity has been evaluated by measuring their capacity to activate or inactivate the human androgen receptor (hAR), glucocorticoid receptor (hGR) and mineralocorticoid receptor (hMR). Transfection assays were performed in HEK 293T cells by using expression vectors for the hAR, hGR and hMR and the GRE2-Luc reporter used for the PR transactivation assays.

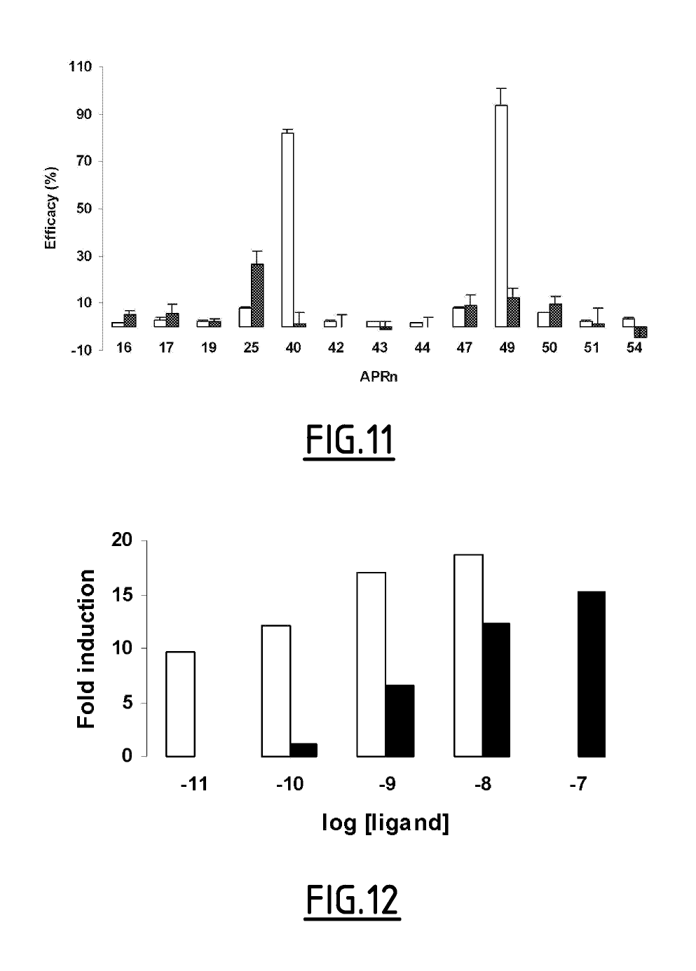

Except for APR40 and APR49 which are high potent hAR agonists, the selected APRn (APR16, APR17, APR19, APR42, APR43, APR44, APR47, APR50, APR51 and APR54) display a low agonist efficacy towards hAR (FIG. 11). Furthermore, the selected APRn were nearly unable to inactivate hAR (FIG. 11). Interestingly, the selected APRn were nearly unable to activate or inactivate hGR and hMR at the concentration of 10⁶ M (Table 1). Thus, APR42 and APR54 which are lacking 3-substituent and the APR16, APR17, APR19, APR43, APR47, APR50, APR51 which are characterized by a fluorine at the 3 position are selective hPR antagonists.

TABLE 1
APRn efficiency in HEK293T cells transiently expressing hMR or hGR

| | MR | | GR | |
|---|---|---|---|---|
| | Agonist | Antagonist | Agonist | Antagonist |
| | efficacy (%) | efficacy (%) | efficacy (%) | efficacy (%) |
| APR16 | 0.53 ± 0.05 | −2.7 ± 3.3 | 0.25 ± 0.02 | 0.1 ± 2.2 |
| APR19 | 0.57 ± 0.07 | −1.0 ± 3.5 | 0.26 ± 0.05 | −3.5 ± 1.4 |
| APR25 | 0.53 ± 0.07 | 2.7 ± 1.9 | 0.25 ± 0.03 | 1.0 ± 3.5 |
| APR40 | 0.57 ± 0.05 | 0.4 ± 2.6 | 0.30 ± 0.06 | 5.5 ± 3.9 |
| APR42 | 0.57 ± 0.05 | 0.4 ± 2.6 | 0.22 ± 0.07 | 4.4 ± 3.3 |
| APR43 | — | — | 0.17 ± 0.05 | −9.0 ± 1.7 |
| APR44 | 0.47 ± 0.10 | 1.6 ± 5.5 | 0.20 ± 0.10 | 2.0 ± 2.8 |
| APR47 | 0.50 ± 0.14 | 2.2 ± 2.1 | 0.16 ± 0.02 | 1.5 ± 4.3 |
| APR50 | — | — | 0.25 ± 0.10 | 7.4 ± 1.9 |
| APR51 | — | — | 0.29 ± 0.08 | 19.3 ± 7.3 |
| APR54 | 0.53 ± 0.11 | 3.4 ± 6.2 | 0.15 ± 0.02 | −0.1 ± 3.3 |

Recruitment of Transcriptional Co-Regulators by PR Upon Ligand Binding

Recruitment of Transcriptional Co-Repressor

Both agonist- and antagonist-bound PR regulate gene transcription with the assistance of transcriptional co-repressors (NCoR, SMRT) or co-activators (namely, TIF-2). To characterize the mechanism by which APRn inactivate hPRB, the capacity of hPRB to recruit transcriptional co-regulators upon APRn binding has been evaluated. With this aim, mammalian two-hybrid assays in HEK293T cells have been performed by using two series of fusion proteins: (i) the VP16 activation domain of herpes simplex virus fused to the entire hPRB and (ii) the GAL4 DNA-binding domain (DBD) fused to the receptor interacting domain (RID) of corepressors (NCoR and SMRT), the GAL4 DBD fused to the 1-867 sequence of the co-activator TIF2 (corresponding to the Nter and RID domains), the GAL4 DBD fused to the 1-623 sequence of TIF2 (corresponding to the Nter domain).

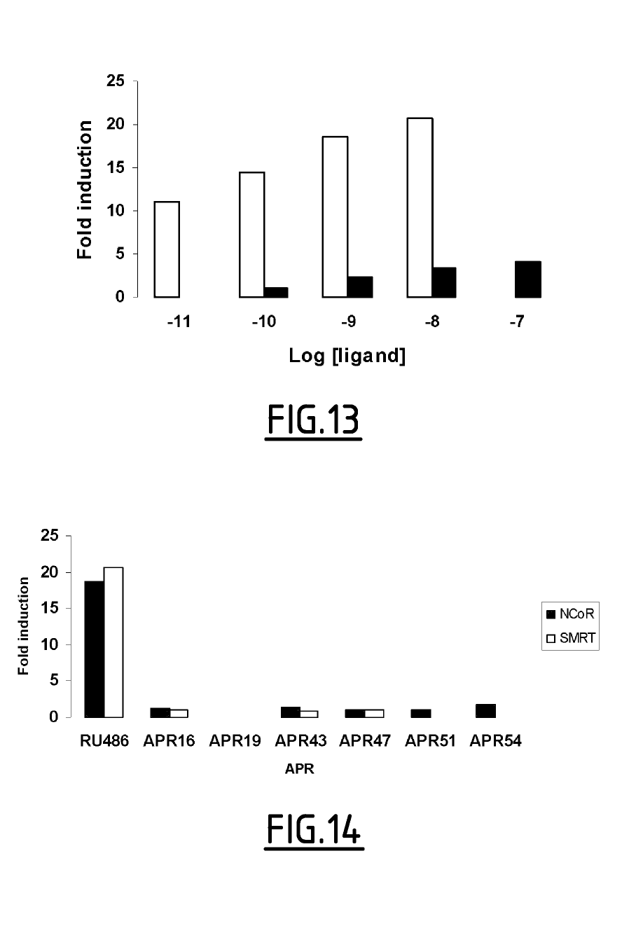

The two hybrid assays revealed that RU486 promoted a dose-dependent binding to hPRB of both NCoR (FIG. 12) and SMRT (FIG. 13). Under the same experimental conditions NCoR, but not SMRT, was recruited upon progesterone binding to hPRB. These findings confirm previous reports, highlighting the recruitment of co-repressors upon the binding of RU486, and to a less extend upon progesterone binding. Interestingly, hPRB was unable to recruit the co-repressors NCoR and SMRT upon the binding of APR16, APR43, APR47, APR51 and APR54 (FIG. 14). These results strongly suggest that the complex between hPRB and an APRn does not adopt the conformation able to bind transcriptional co-repressor and/or is not stable enough to recruit co-repressors.

Recruitment of Transcriptional Co-Activator by PR

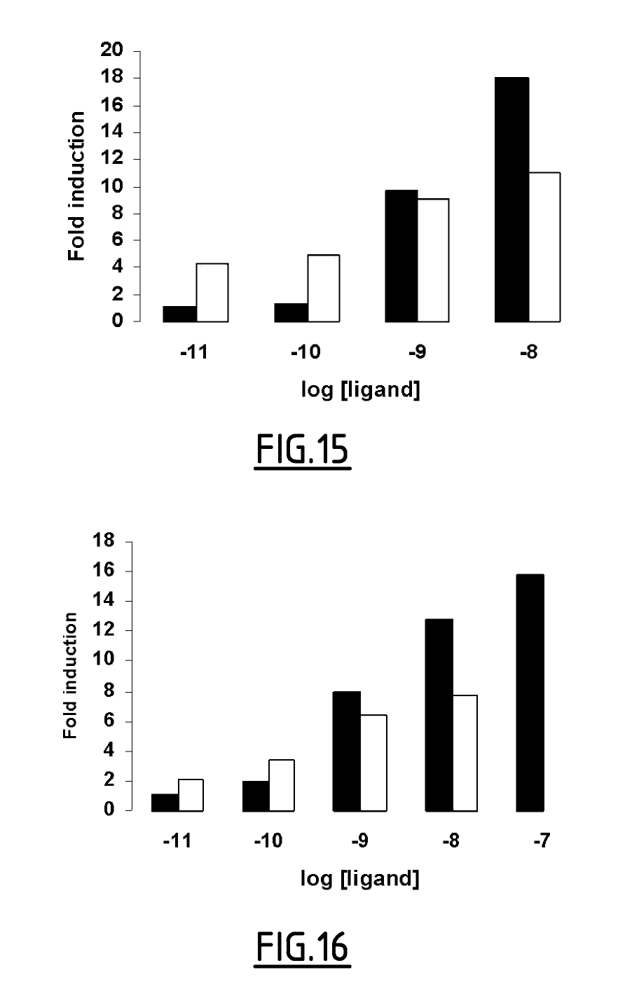

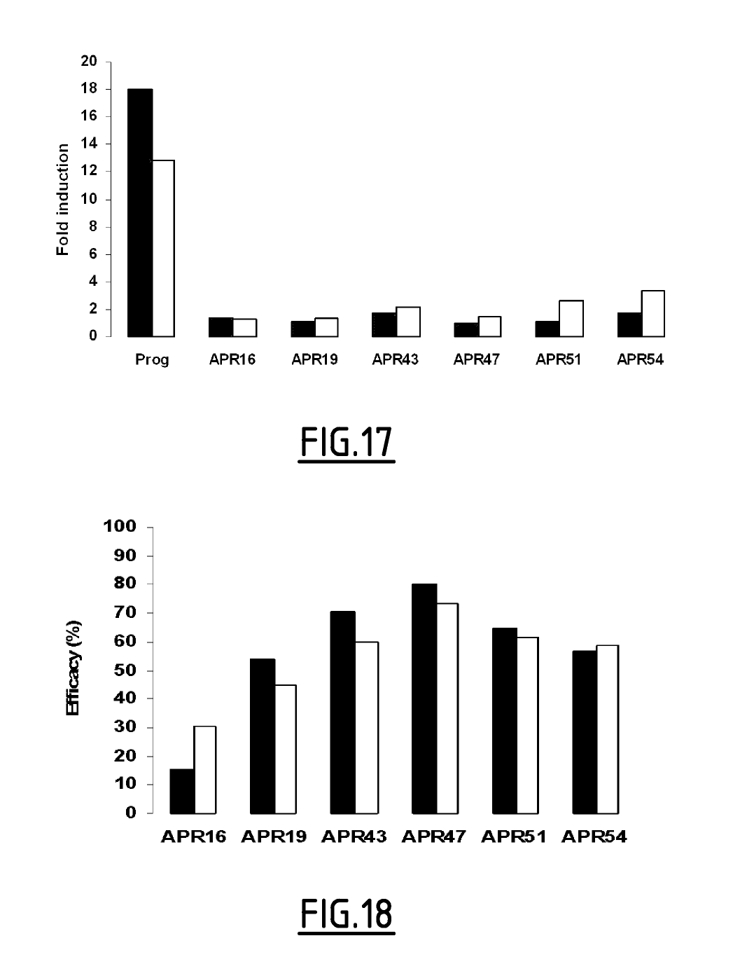

Further, the capacity of hPRB to bind the co-activator TIF-2 was examined. The two-hybrid assays were performed with the TIF-2 sequence encompassing its N-ter region alone (TIF-2-Nter) or the sequence including the N-ter and the RID regions (TIF-2-NterRID). Indeed, the N-ter and the RID regions have been shown to interact in a different manner with agonist-PR complexes (Wand, D. et al. Biochemistry, 2007,46, 8036). Two-hybrid assays revealed that progesterone and also RU486 promoted a dose-dependent binding of both TIF-2 sequences (FIGS. 15 and 16). Nevertheless, TIF-2 binding was lower upon RU486 compared to progesterone. These results confirm that agonist- and antagonist-bound hPRB are both able to recruit co-activators. Interestingly, hPRB was unable to recruit the two TIF-2 sequences upon the binding of APR16, APR19, APR43, APR47, APR51 and APR54. Moreover, these APRn were able to inhibit the progesterone induced TIF-2 recruitment by hPRB. These results are illustrated in the FIG. 18, which represents the efficacy of the APRn to inhibit the progesterone induced TIF-2 recruitment.

From these findings, it can be proposed that the APRn are able to inhibit the binding of progesterone to hPRB. They form with hPRB unstable complexes which are unable to recruit both transcriptional co-activators and co-repressors. Thus, APRn may be classified as passive antagonists, constituting a novel generation of hPRB antagonists.

APR-19 Efficiency on Inhibiting Progesterone Activity

In this model, progesterone administration prevents the estrogendependent proliferation of endometrial cells, while in contrast, the anti-proliferative effect of progesterone could be abolished by antagonist ligands. Following this protocol APR-19 inhibited the anti-proliferative effects of progesterone on E2-induced endometrial proliferation.

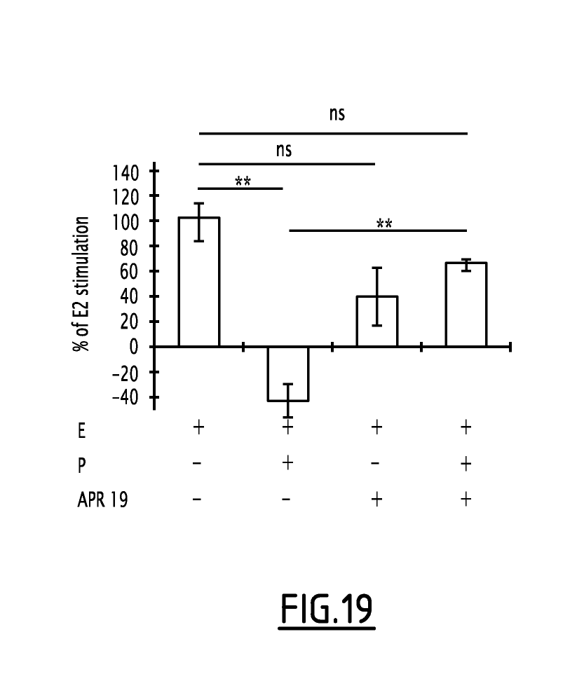

These results are illustrated in the FIG. 19, which represents the efficiency of APR-19 to inhibit the anti-proliferative effects of progesterone on E2-induced endometrial proliferation.

1. A method of providing progesterone receptor antagonist activity to a subject in need thereof, comprising
administering to said subject a therapeutically effective amount of a compound of formula (I): 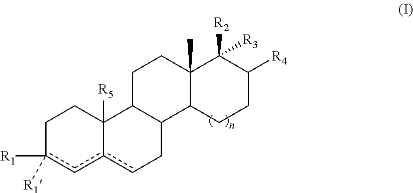
wherein
n is 0 or 1; 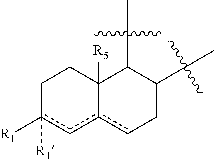
is selected from (Ia), (Ib), (Ic), (Id), (Ie), (If) and (Ig): 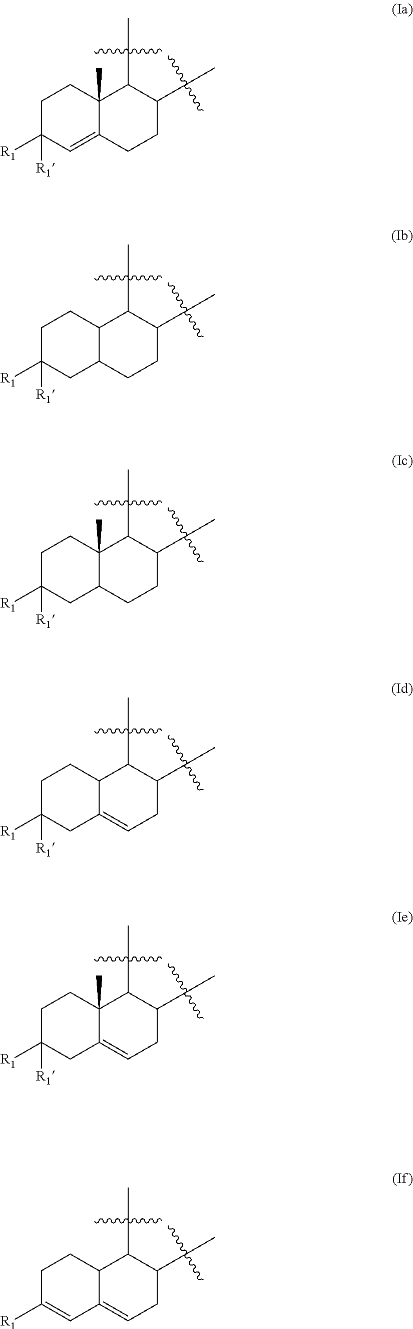 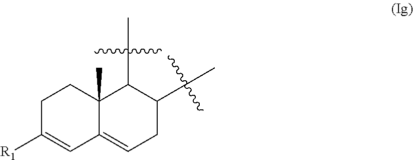
R₁ and R₁′ are each independently selected from H, OR₆, and halogen, or a 5 to 7 membered heterocyclyl group;
R₂ and R₃ are each independently selected from H, C(O)R₈, OR₇, halogen, CH(OR₇)(R₈), C(OR₆)(C≡CR₆)(R₈) and C≡CR₆,
provided that when R₂ is OH, R₃ cannot be H,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms;
or its pharmaceutically acceptable salts, hydrates or hydrated salts or its polymorphic crystalline structures, racemates, diastereoisomers or enantiomers, with the exclusion of the compounds: 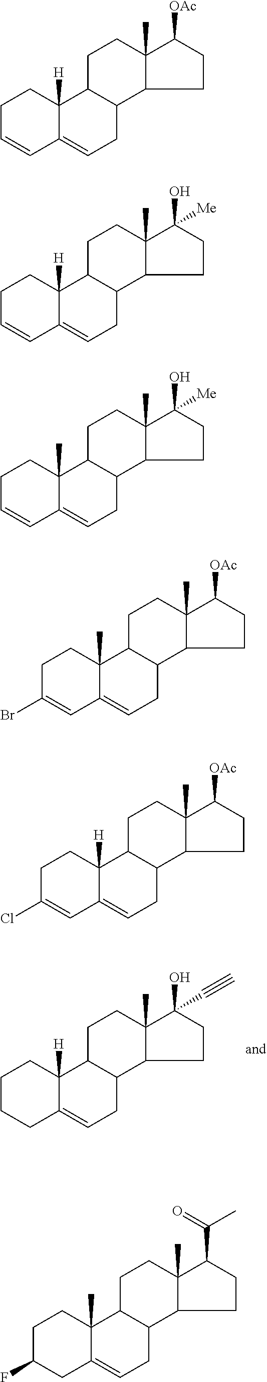
.

2. The method of claim 1, wherein said compound is the compound of formula (I):
wherein
n is 0 or 1; 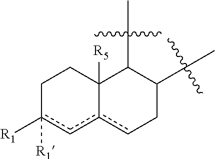
is selected from (Ia), (Ib), (Ic), (Id), (Ie), (If) and (Ig): 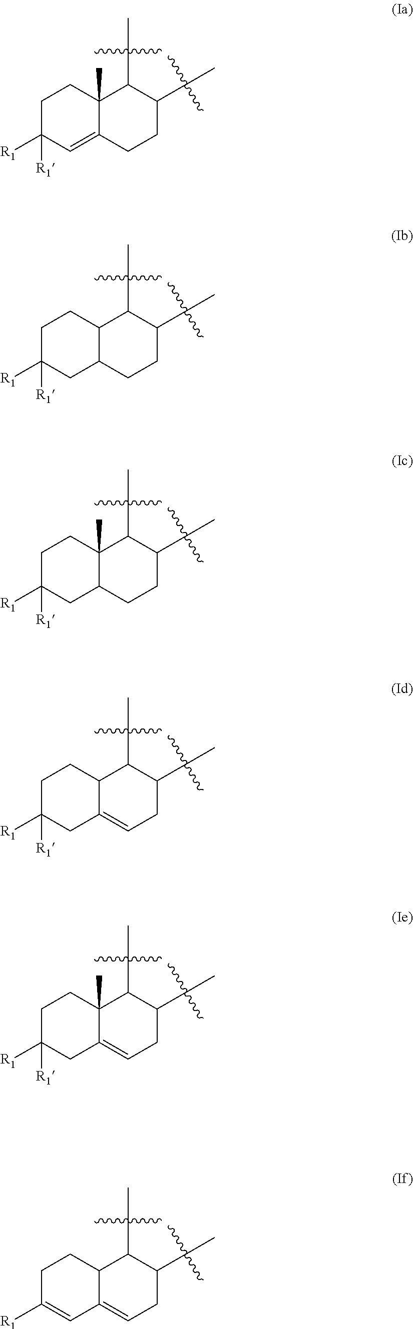 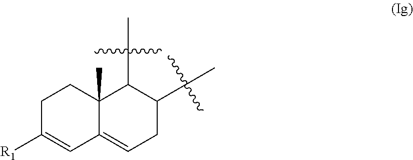
R₁ and R₁′ are each independently selected from H, OR₆, and halogen, or a 5 to 7 membered heterocyclyl group;
R₂ and R₃ are each independently selected from H, C(O)R₈, OR₇, halogen, CH(OR₇)(R₈), C(OR₆)(C≡CR₆)(R₈) and C≡CR₆,
provided that when R₂ is OH, R₃ cannot be H,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms;
or its pharmaceutically acceptable salts, hydrates or hydrated salts or its polymorphic crystalline structures, racemates, diastereoisomers or enantiomers,
and wherein said method is a method of preventing or treating a pathology involving progesterone receptor.

3. The method of claim 1, wherein said compound is the compound of formula (I):
wherein
n is 0 or 1; 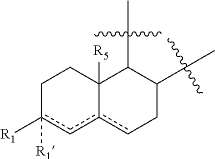
is selected from (Ia), (Ib), (Ic), (Id), (Ie), (If) and (Ig): 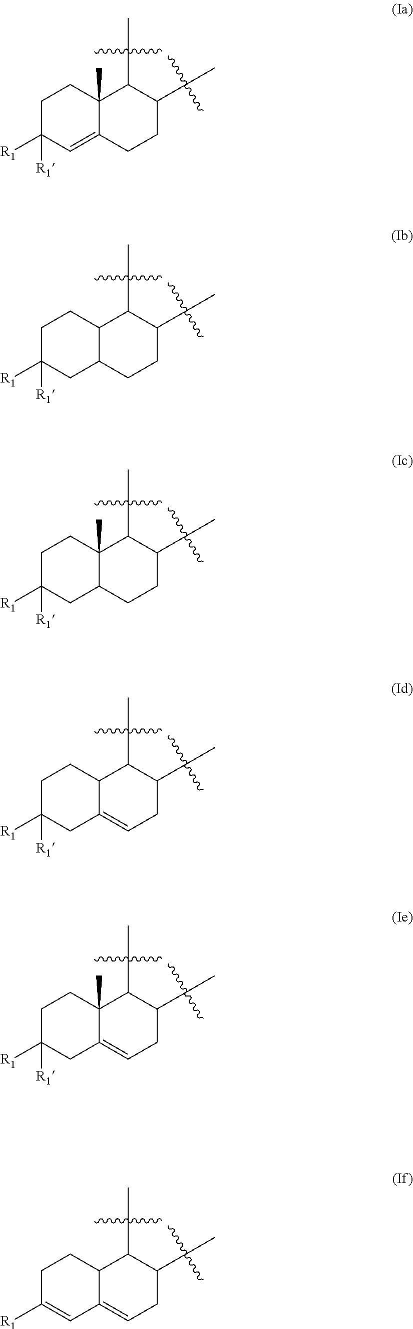 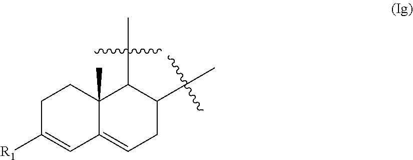
R₁ and R₁′ are each independently selected from H, OR₆, and halogen, or a 5 to 7 membered heterocyclyl group;
R₂ and R₃ are each independently selected from H, C(O)R₈, OR₇, halogen, CH(OR₇)(R₈), C(OR₆)(C≡CR₆)(R₈) and C≡CR₆,
provided that when R₂ is OH, R₃ cannot be H,
R₄ is H or an alkyl group comprising from 1 to 6 carbon atoms;
R₆ is H or an alkyl group comprising from 1 to 6 carbon atoms;
R₇ is H, an alkyl group comprising from 1 to 6 carbon atoms, or a group C(O)R₉, wherein R₉ is an alkyl group comprising from 1 to 6 carbon atoms;
R₈ is an alkyl group comprising from 1 to 6 carbon atoms;
or its pharmaceutically acceptable salts, hydrates or hydrated salts or its polymorphic crystalline structures, racemates, diastereoisomers or enantiomers, with the exclusion of the compounds: 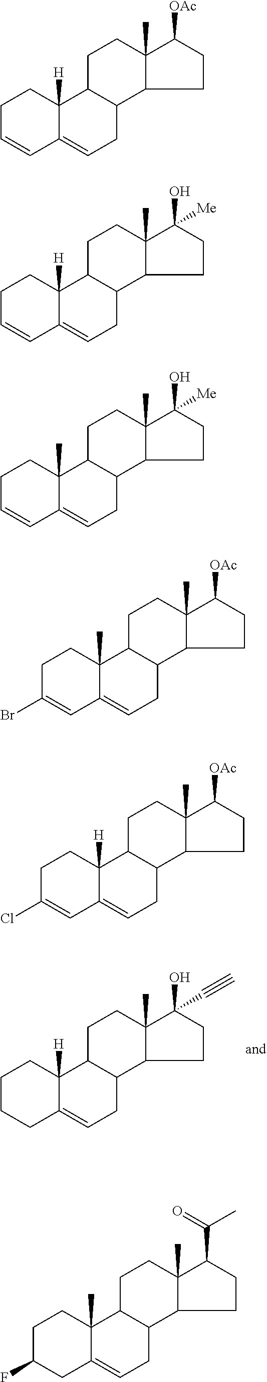
and wherein said method is used for estrogen-free contraception, emergency contraception, antigestation, or to provide an abortifacient.

4. The method of claim 1, wherein R₃ is H and R₂ is selected from C(O)R₈, OR′₇, halogen, CH(OR₇)(R₈), C(OR₆)(C≡CR₆)(R₈) and C≡CR₆, wherein:
R₆ is H or an alkyl group comprising from 1 to 6 carbon atoms;
R₇ is H or an alkyl group comprising from 1 to 6 carbon atoms, or a group C(O)R₉,
R′₇ is an alkyl group comprising from 1 to 6 carbon atoms, or a group C(O)R₉,
R₈ is an alkyl group comprising from 1 to 6 carbon atoms; and
R₉ is an alkyl group comprising from 1 to 6 carbon atoms.

5. The method of claim 4, wherein R₂ is OAc.

6. The method of claim 4, wherein R₂ is selected from COCH₃, CH(CH₃)(OH) and CH(CH₃)(OAc).

7. The method of claim 4, wherein R₂ is C(C≡CH)(CH₃)(OH).

8. The method of claim 2, wherein R₂ is OH and R₃ is C═CR₆.

9. The method of claim 2, wherein n is 0 and R₁ and R′₁ are H.

10. The method of claim 2, wherein said compound is the compound of formula (I′): 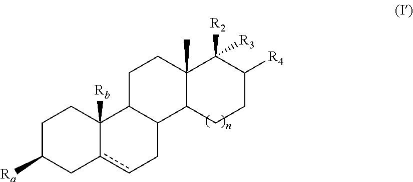
wherein n, R₂, R₃, and R₄ are as defined in claim 2, and 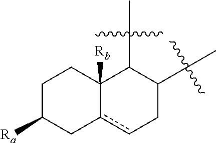
is selected from (IIa′), (IIb′), (IIc′) and (IId′): 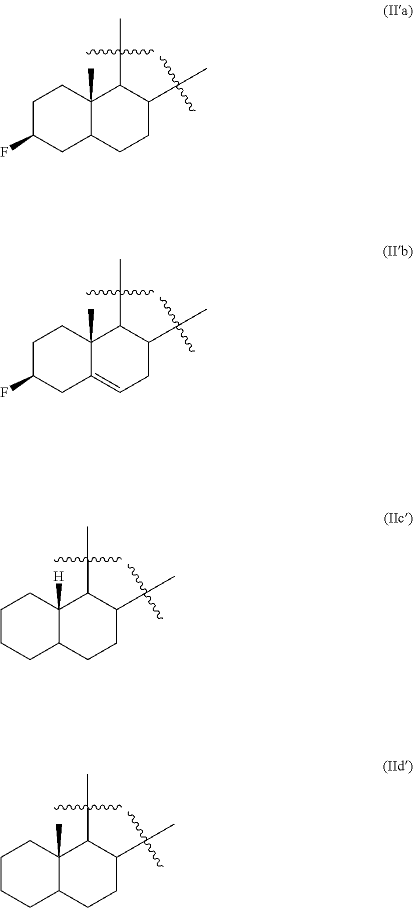 [SMILES: *C1CCC2C[C@@H](F)CC[C@]2(C)C1* ; *C1CC=C2C[C@@H](F)CC[C@]2(C)C1* ; *C1CCC2CCCC[C@@H]2C1* ; *C1CCC2CCCC[C@]2(C)C1*]
.

11. The method of claim 2, wherein said compound is the compound of formula (II): 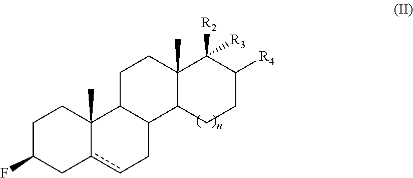
wherein n, R₂, R₃, and R₄ are as defined in claim 2, and 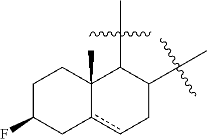
is selected from (Ia), and (Ib): 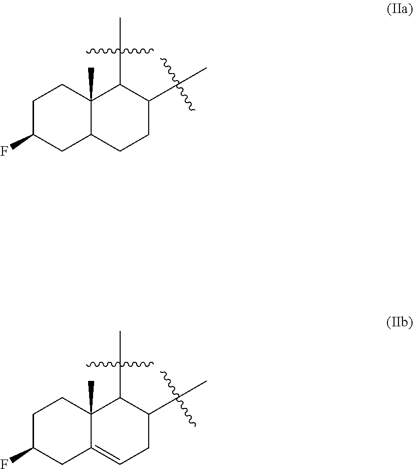 [SMILES: *C1CCC2C[C@@H](F)CC[C@]2(C)C1* ; *C1CC=C2C[C@@H](F)CC[C@]2(C)C1*]
.

12. The method of claim 2, wherein said compound is the compound of formula (II-1-2): 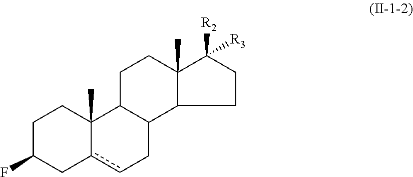
wherein R₂ and R₃ are as defined in claim 2,
and 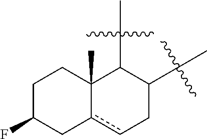
is as defined in claim 11.

13. The method of claim 2, wherein said compound is the compound of formula (II-2′): 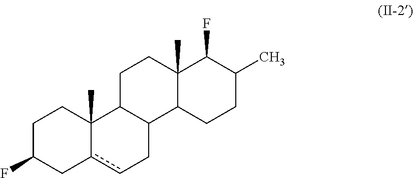
wherein 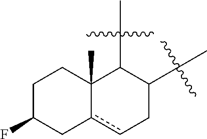
is as defined in claim 11.

14. A compound of formula (II-2): 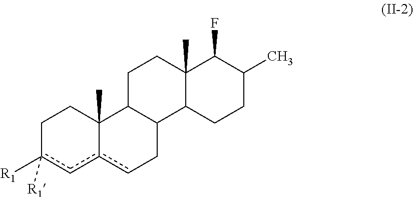
wherein: 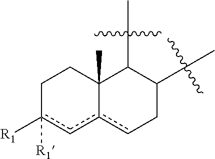
is selected from (II-2a), (II-2b), (II-2c), and (II-2d): 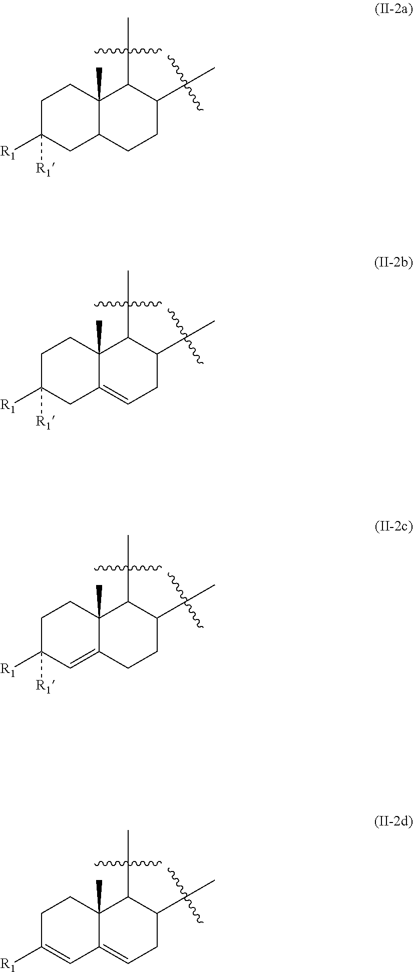
R₁ and R₁′ are each independently selected from H, OR₆, and halogen, or together with the carbon atom to which they are attached form a group C═O, or a 5 to 7 membered heterocyclyl group;
provided that when 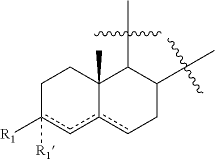
is (II-2d), R₁ cannot be C═O; and
R₆ is H or an alkyl group comprising from 1 to 6 carbon atoms,
or its pharmaceutically acceptable salts, hydrates or hydrated salts or its polymorphic crystalline structures, racemates, diastereoisomers or enantiomers.

15. A compound of formula (II-3) 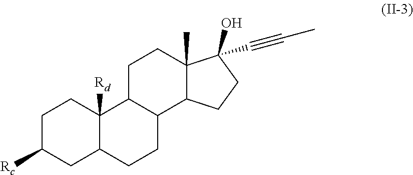
wherein: 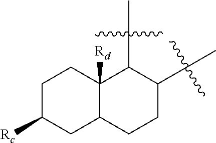
is selected from (II-3a), (II-3b) and (II-3c): 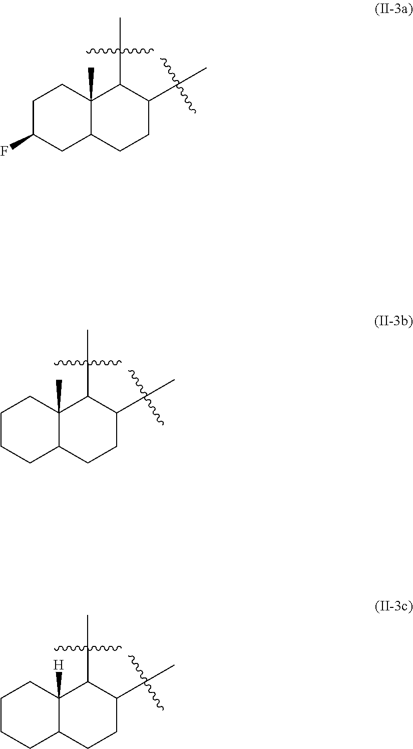 [SMILES: *C1CCC2C[C@@H](F)CC[C@]2(C)C1* ; *C1CCC2CCCC[C@]2(C)C1* ; *C1CCC2CCCC[C@@H]2C1*]
or its pharmaceutically acceptable salts, hydrates or hydrated salts or its polymorphic crystalline structures, racemates, diastereoisomers or enantiomers.

16. A method of providing progesterone receptor antagonist activity to a subject in need thereof, comprising
administering to said subject a therapeutically effective amount of a compound of formula (II-2) according to claim 14.

17. A method of preventing and/or treating cancer or uterine pathologies in a subject in need thereof, comprising
administering to said subject a therapeutically effective amount of a compound of formula (II-2) according to claim 14.

18. A method of providing progesterone receptor antagonist activity to a subject in need thereof, comprising
administering to said subject a therapeutically effective amount of a compound of formula (II-3) according to claim 15.

19. A method of preventing and/or treating cancer or uterine pathologies in a subject in need thereof, comprising
administering to said subject a therapeutically effective amount of a compound of formula (II-3) according to claim 15.

20. A compound having one of the following formulae:

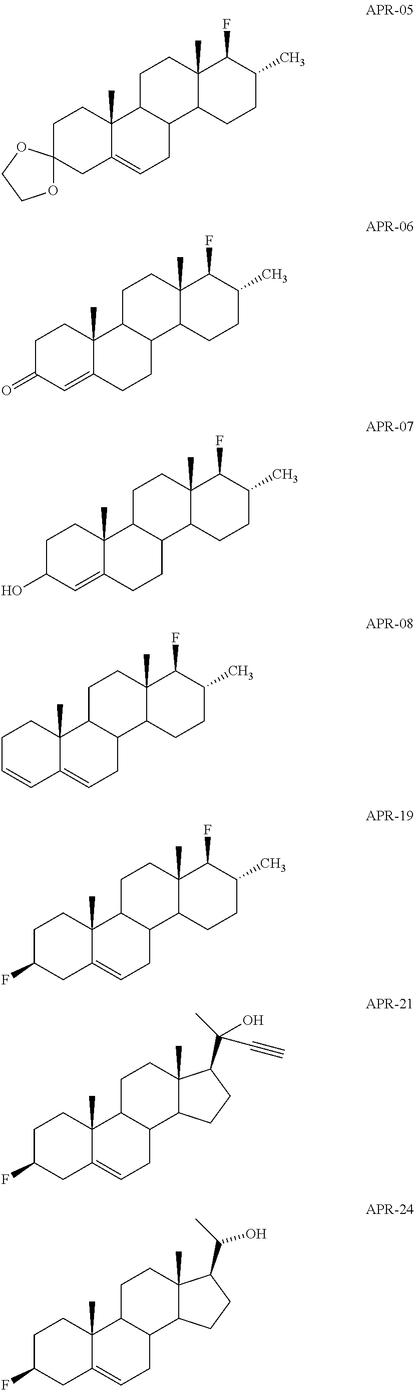

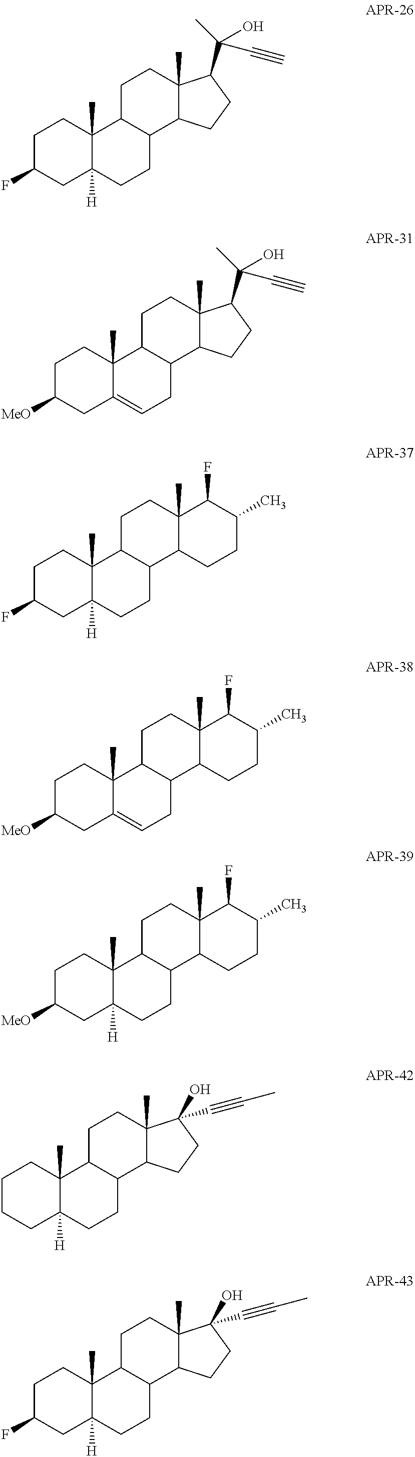

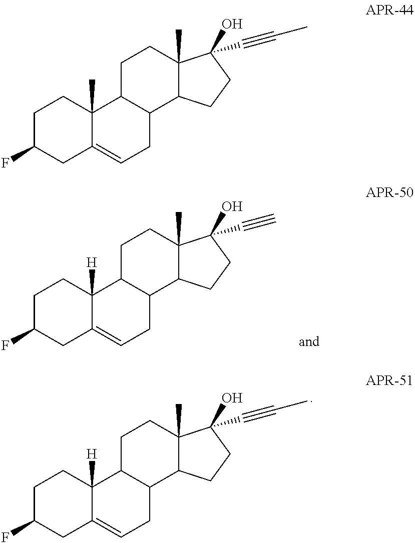 [SMILES: CC#C[C@]1(O)CCC2C3CC=C4C[C@@H](F)CC[C@]4(C)C3CC[C@@]21C ; C#C[C@]1(O)CCC2C3CC=C4C[C@@H](F)CC[C@@H]4C3CC[C@@]21C ; CC#C[C@]1(O)CCC2C3CC=C4C[C@@H](F)CC[C@@H]4C3CC[C@@]21C]

21. A compound having one of the following formulae:

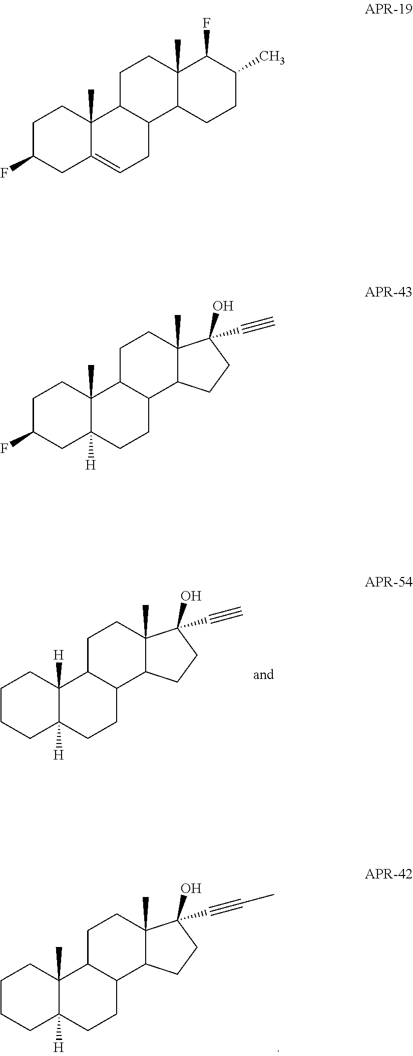 [SMILES: C[C@@H]1CCC2C3CC=C4C[C@@H](F)CC[C@]4(C)C3CC[C@]2(C)[C@H]1F ; C#C[C@]1(O)CCC2C3CC[C@H]4C[C@@H](F)CC[C@]4(C)C3CC[C@@]21C ; C#C[C@]1(O)CCC2C3CC[C@H]4CCCC[C@@H]4C3CC[C@@]21C ; CC#C[C@]1(O)CCC2C3CC[C@H]4CCCC[C@]4(C)C3CC[C@@]21C]

22-23. (canceled)

24. The method of claim 1, wherein said method is used for estrogen-free contraception, emergency contraception, antigestation, or to provide an abortifacient.

25. The method of claim 2, wherein said pathology involving progesterone receptor is cancer or a uterine pathology.

26. The method of claim 2, wherein R₃ is H and R₂ is selected from C(O)R₈, OR′₇, halogen, CH(OR₇)(R₈), C(OR₆)(C≡CR₆)(R₈) and C≡CR₆, wherein:
R₆ is H or an alkyl group comprising from 1 to 6 carbon atoms;
R₇ is H or an alkyl group comprising from 1 to 6 carbon atoms, or a group C(O)R₉,
R′₇ is an alkyl group comprising from 1 to 6 carbon atoms, or a group C(O)R₉,
R₈ is an alkyl group comprising from 1 to 6 carbon atoms; and
R₉ is an alkyl group comprising from 1 to 6 carbon atoms.

27. The method of claim 26, wherein R₂ is OAc.

28. The method of claim 26, wherein R₂ is selected from COCH₃, CH(CH₃)(OH) and CH(CH₃)(OAc).

29. The method of claim 26, wherein R₂ is C(C≡CH)(CH₃)(OH).

30. The method of claim 8, wherein R₆ is H or CH₃.

31. The method of claim 1, wherein said compound is selected from the group consisting of

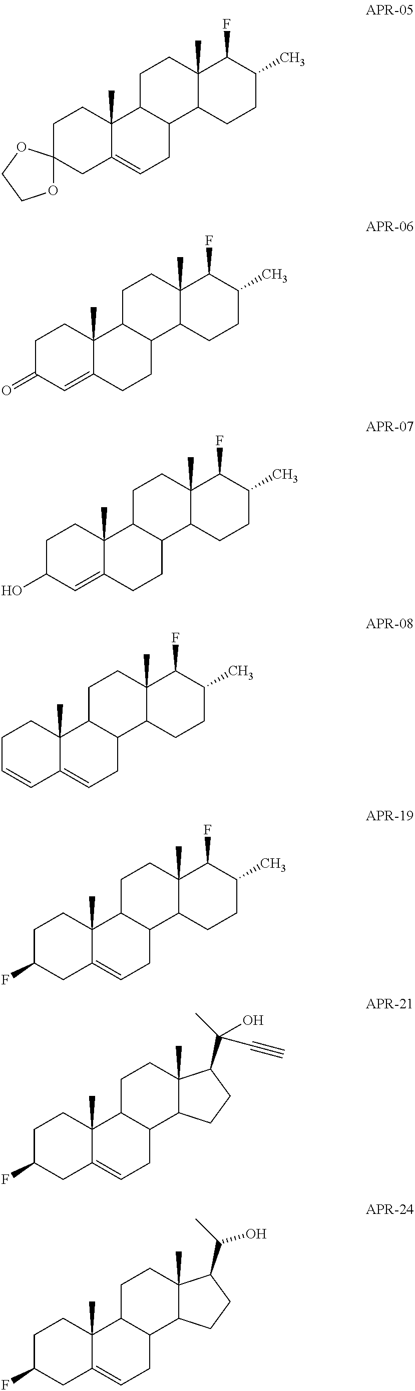

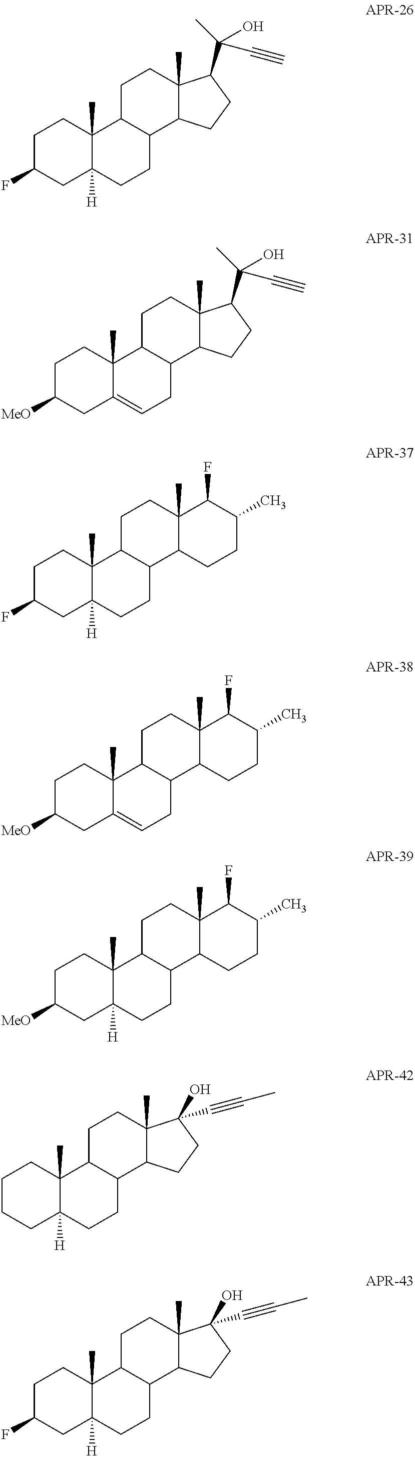

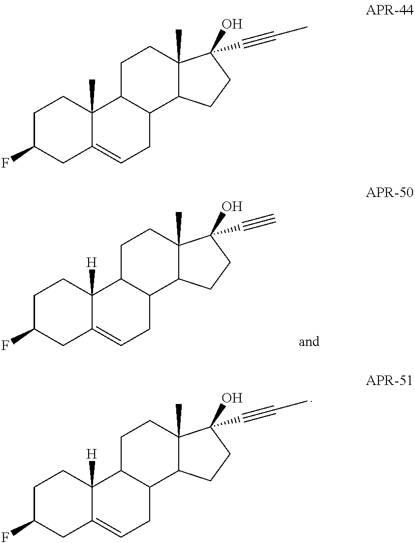 [SMILES: CC#C[C@]1(O)CCC2C3CC=C4C[C@@H](F)CC[C@]4(C)C3CC[C@@]21C ; C#C[C@]1(O)CCC2C3CC=C4C[C@@H](F)CC[C@@H]4C3CC[C@@]21C ; CC#C[C@]1(O)CCC2C3CC=C4C[C@@H](F)CC[C@@H]4C3CC[C@@]21C]

32. The method of claim 31, wherein said compound is selected from

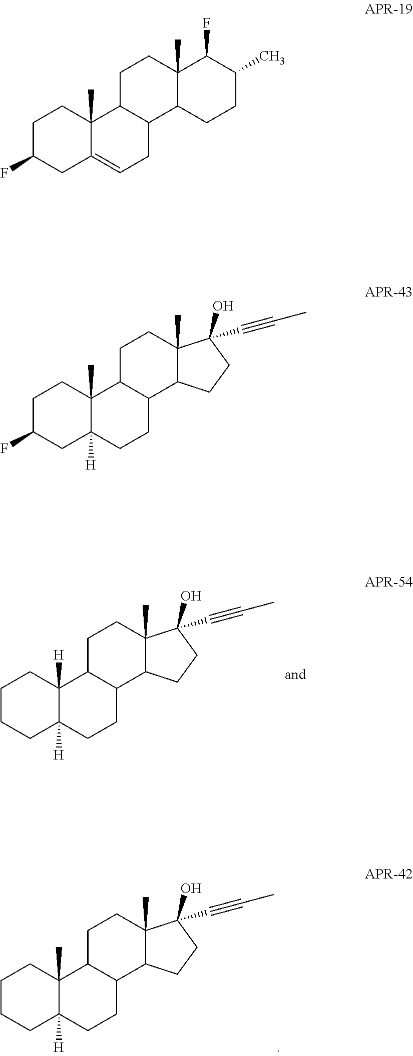 [SMILES: C[C@@H]1CCC2C3CC=C4C[C@@H](F)CC[C@]4(C)C3CC[C@]2(C)[C@H]1F ; CC#C[C@]1(O)CCC2C3CC[C@H]4C[C@@H](F)CC[C@]4(C)C3CC[C@@]21C ; CC#C[C@]1(O)CCC2C3CC[C@H]4CCCC[C@@H]4C3CC[C@@]21C ; CC#C[C@]1(O)CCC2C3CC[C@H]4CCCC[C@]4(C)C3CC[C@@]21C]

33. The method of claim 25, wherein said compound is selected from the group consisting of

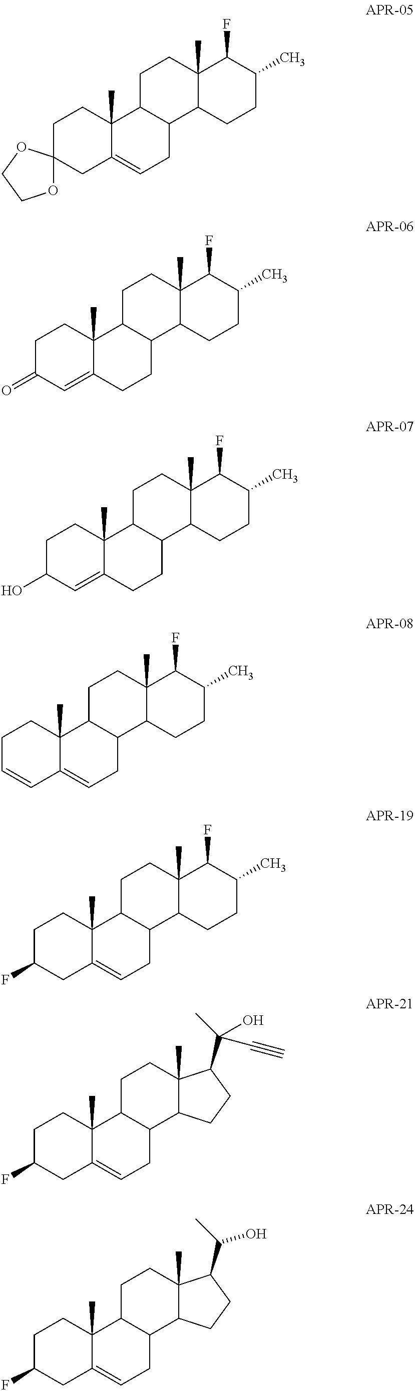

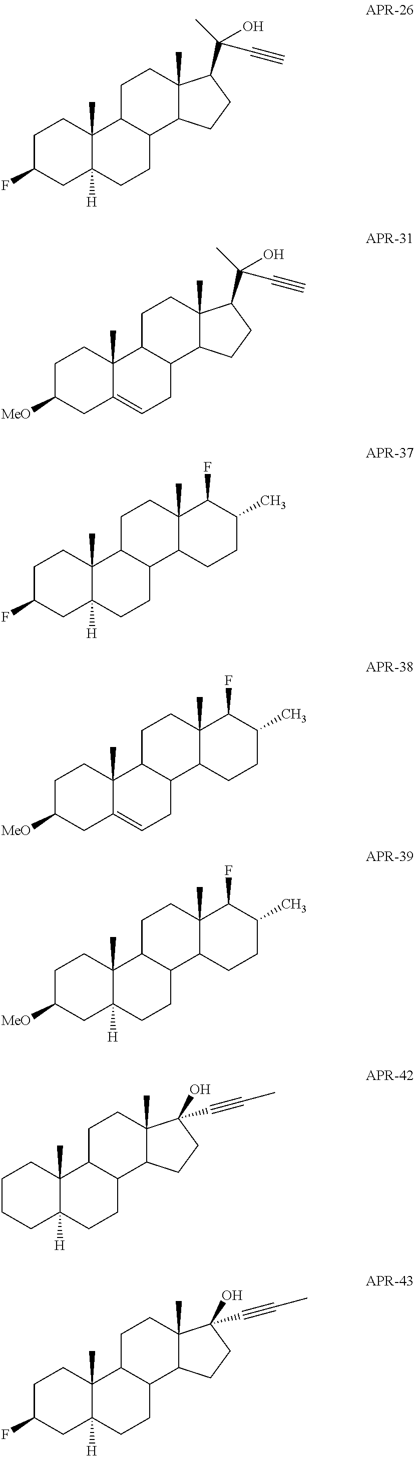

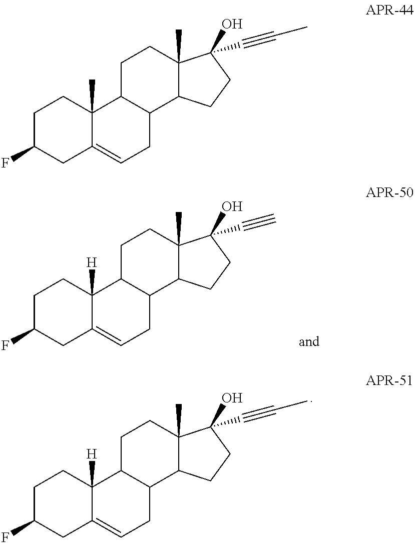 [SMILES: CC#C[C@]1(O)CCC2C3CC=C4C[C@@H](F)CC[C@]4(C)C3CC[C@@]21C ; C#C[C@]1(O)CCC2C3CC=C4C[C@@H](F)CC[C@@H]4C3CC[C@@]21C ; CC#C[C@]1(O)CCC2C3CC=C4C[C@@H](F)CC[C@@H]4C3CC[C@@]21C]

34. The method of claim 33, wherein said compound is selected from 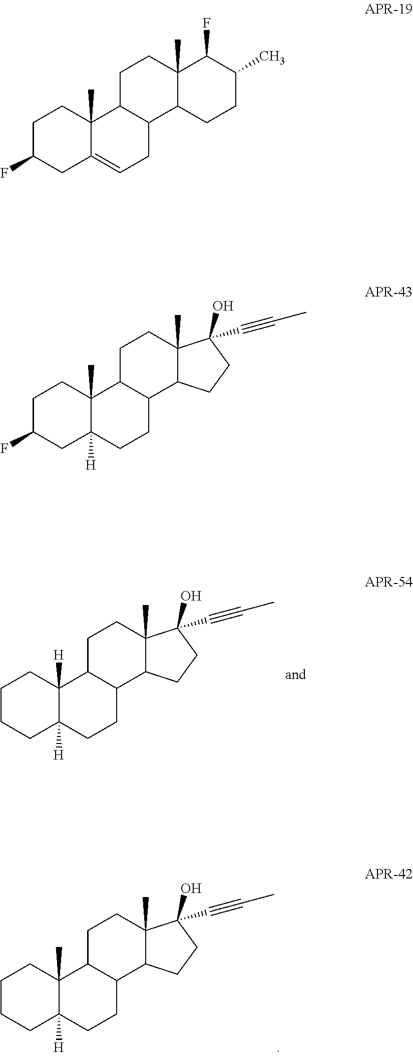 [SMILES: C[C@@H]1CCC2C3CC=C4C[C@@H](F)CC[C@]4(C)C3CC[C@]2(C)[C@H]1F ; CC#C[C@]1(O)CCC2C3CC[C@H]4C[C@@H](F)CC[C@]4(C)C3CC[C@@]21C ; CC#C[C@]1(O)CCC2C3CC[C@H]4CCCC[C@@H]4C3CC[C@@]21C ; CC#C[C@]1(O)CCC2C3CC[C@H]4CCCC[C@]4(C)C3CC[C@@]21C]
